(19) United States
(12) Patent Application Publication
(10) Pub. No.: US 2023/0099970 A1
(43) Pub. Date: Mar. 30, 2023

(54) METHOD AND SYSTEM FOR ULTRASONIC IMAGING

(71) Applicant: WUHAN UNITED IMAGING HEALTHCARE CO., LTD., Wuhan (CN)

(72) Inventors: Rui LUO, Wuhan (CN); Zhixin TIAN, Wuhan (CN); Hao LING, Wuhan (CN)

(73) Assignee: WUHAN UNITED IMAGING HEALTHCARE CO., LTD., Wuhan (CN)

(21) Appl. No.: 17/810,330

(22) Filed: Jun. 30, 2022

Related U.S. Application Data
(63) Continuation of application No. PCT/CN2021/138274, filed on Dec. 15, 2021.

(30) Foreign Application Priority Data
Sep. 24, 2021 (CN) 202111124331.2
Oct. 26, 2021 (CN) 202111250486.0

(51) Int. Cl. A61B 8/00; A61B 8/14; G01S 15/89; G01S 7/52

(52) U.S. Cl. CPC A61B 8/54; A61B 8/14; G01S 15/8995; G01S 7/52085

(57) ABSTRACT

The present disclosure discloses methods, systems, and computer readable mediums for ultrasonic imaging. The method may include determining a target imaging mode according to information related to one or more imaging demands. The method may further include obtaining a target imaging result by performing an imaging operation according to the target imaging mode, wherein the one or more imaging demands may at least include a demand related to an imaging quality and/or a frame rate, the target imaging mode may include a first target imaging mode and/or a second target imaging mode, the first target imaging mode may be configured to perform an optimized imaging for a local imaging region, and/or the second target imaging mode may be configured to utilize a hybrid wave with at least two transmission beam types and/or at least two transmission frequencies for imaging.

### CROSS-REFERENCE TO RELATED APPLICATIONS

This application is a continuation of International Patent Application No. PCT/CN2021/138274, filed on Dec. 15, 2021, which claims priority to Chinese Patent Application No. 202111124331.2, filed on Sep. 24, 2021, and Chinese Patent Application No. 202111250486.0, filed on Oct. 26, 2021, the contents of each of which are hereby incorporated by reference.

### TECHNICAL FIELD

The present disclosure relates to ultrasonic medical technology field, and more particularly, relates to method, system, device, and computer readable medium for ultrasonic imaging.

### BACKGROUND

Ultrasonic medicine may combine technologies such as acoustics, medicine, optics, and electronics, and apply to ultrasonic diagnosis, ultrasonic therapy, etc. For example, the ultrasonic diagnosis may apply ultrasonic detection technology to the human body, which may give hints or guidance for the discovery of diseases by measuring data and morphology of physiological or tissue structure. The ultrasonic diagnosis may be used as a noninvasive, painless, convenient, and intuitive effective examination method, especially B-ultrasonic examination, which is widely used.

However, in ultrasonic medical applications such as the ultrasonic diagnosis, ultrasonic imaging may be a core and key step. The ultrasonic imaging may transmit ultrasonic wave from ultrasonic probe to the object to be inspected, and generate an ultrasonic image according to ultrasonic echo signals. The imaging effect of the ultrasonic imaging is directly related to the execution efficiency of the ultrasonic medical applications such as the ultrasonic diagnosis. Therefore, it is also a key field of medical improvement.

### SUMMARY

An aspect of the present disclosure provides a method for ultrasonic imaging. The method may be implemented on a computing device including at least one processor and a computer-readable storage device. The method for ultrasonic imaging may include determining a target imaging mode according to information related to one or more imaging demands. The method for ultrasonic imaging may also include obtaining a target imaging result by performing an imaging operation according to the target imaging mode, wherein the one or more imaging demands at least include a demand related to an imaging quality and/or a frame rate, the target imaging mode may include a first target imaging mode and/or a second target imaging mode, the first target imaging mode may be configured to perform an optimized imaging for a local imaging region and/or the second target imaging mode may be configured to utilize a hybrid wave with at least two transmission beam types and/or at least two transmission frequencies for imaging.

Another aspect of the present disclosure provides a method for ultrasonic imaging. The method may be implemented on a computing device including at least one processor and a computer-readable storage device. The method for ultrasonic imaging may include determining a region of interest in an initial image, the initial image includes a global imaging region. The method for ultrasonic imaging may also include determining imaging condition data according to the region of interest. The method for ultrasonic imaging may also include determining a target optimized imaging mode based on the imaging condition data, and triggering an imaging operation in the target optimized imaging mode to generate an optimized image, the target optimized imaging mode including a first optimized imaging mode or a second optimized imaging mode, wherein the first optimized imaging mode may be configured to perform an operation for adjusting a transmission parameter of a transmission covering the global imaging region, the second optimized imaging mode may be configured to perform a synchronous operation of an enhanced imaging covering the region of interest and a non-enhanced imaging covering the global imaging region.

Another aspect of the present disclosure provides a method for ultrasonic imaging. The method may be implemented on a computing device including at least one processor and a computer-readable storage device. The method for ultrasonic imaging may include determining a hybrid wave imaging mode according to information related to one or more imaging demands. The method for ultrasonic imaging may also include obtaining an imaging result by performing a hybrid wave imaging operation according to the hybrid wave imaging mode, wherein the one or more imaging demands may at least include a demand related to an image quality and/or a frame rate, the hybrid wave imaging operation may at least include utilizing a hybrid wave with at least two transmission beam types and/or at least two transmission frequencies for imaging, and the transmission beam types may at least include a focused wave and/or an unfocused wave.

Another aspect of the present disclosure provides a system for ultrasonic imaging. The system may include at least one storage device including a set of instructions and at least one processor configured to communicate with the at least one storage device. When executing the set of instructions, the at least one processor is configured to direct the system to perform following operations. The operations may include determining a target imaging mode according to information related to one or more imaging demands. The operations may also include obtaining a target imaging result by performing an imaging operation according to the target imaging mode, wherein the one or more imaging demands may at least include a demand related to an imaging quality and/or a frame rate, the target imaging mode may include a first target imaging mode and/or a second target imaging mode, the first target imaging mode may be configured to perform an optimized imaging for a local imaging region and/or the second target imaging mode may be configured to utilize a hybrid wave with at least two transmission beam types and/or at least two transmission frequencies for imaging.

Another aspect of the present disclosure provides a system for ultrasonic imaging. The system may include an imaging mode determination module and an imaging operation module. The imaging mode determination module may be configured to determine a target imaging mode according to information related to one or more imaging demands. The imaging operation module may be configured to obtain a target imaging result by performing an imaging operation according to the target imaging mode, wherein the one or more imaging demands may at least include a demand related to an imaging quality and/or a frame rate, the target imaging mode may include a first target imaging mode and/or a second target imaging mode, the first target imaging mode may be configured to perform an optimized imaging for a local imaging region and/or the second target imaging mode may be configured to utilize a hybrid wave with at least two transmission beam types and/or at least two transmission frequencies for imaging.

Another aspect of the present disclosure provides a non-transitory computer readable medium. The non-transitory computer readable medium may include executable instructions that, when executed by at least one processor, direct the at least one processor to perform a method. The method may include determining a target imaging mode according to information related to one or more imaging demands. The method may also include obtaining a target imaging result by performing an imaging operation according to the target imaging mode, wherein the one or more imaging demands may at least include a demand related to an imaging quality and/or a frame rate, the target imaging mode may include a first target imaging mode and/or a second target imaging mode, the first target imaging mode may be configured to perform an optimized imaging for a local imaging region and/or the second target imaging mode may be configured to utilize a hybrid wave with at least two transmission beam types and/or at least two transmission frequencies for imaging.

Another aspect of the present disclosure provides a system for ultrasonic imaging. The system may include at least one storage device including a set of instructions and at least one processor configured to communicate with the at least one storage device. When executing the set of instructions, the at least one processor is configured to direct the system to perform following operations. The operations may include determining a region of interest in an initial image, the initial image may include a global imaging region. The operations may also include determining imaging condition data according to the region of interest. The operations may also determining a target optimized imaging mode based on the imaging condition data, and triggering an imaging operation in the target optimized imaging mode to generate an optimized image, the target optimized imaging mode including a first optimized imaging mode or a second optimized imaging mode, wherein the first optimized imaging mode may be configured to perform an operation for adjusting a transmission parameter of a transmission covering the global imaging region, the second optimized imaging mode may be configured to perform a synchronous operation of an enhanced imaging covering the region of interest and a non-enhanced imaging covering the global imaging region.

Another aspect of the present disclosure provides a system for ultrasonic imaging. The system may include a region of interest determination module, an imaging condition data determination module, and an optimized image generation module. The region of interest determination module may be configured to determine a region of interest in an initial image, the initial image may include a global imaging region. The imaging condition data determination module may be configured to determine imaging condition data according to the region of interest. The optimized image generation module may be configured to determine a target optimized imaging mode based on the imaging condition data, and triggering an imaging operation in the target optimized imaging mode to generate an optimized image, the target optimized imaging mode including a first optimized imaging mode or a second optimized imaging mode, wherein the first optimized imaging mode may be configured to perform an operation for adjusting a transmission parameter of a transmission covering the global imaging region, the second optimized imaging mode may be configured to perform a synchronous operation of an enhanced imaging covering the region of interest and a non-enhanced imaging covering the global imaging region.

Another aspect of the present disclosure provides a non-transitory computer readable medium. The non-transitory computer readable medium may include executable instructions that, when executed by at least one processor, direct the at least one processor to perform a method. The method may include determining a region of interest in an initial image, the initial image may include a global imaging region. The method may also include determining imaging condition data according to the region of interest. The method may also include determining a target optimized imaging mode based on the imaging condition data, and triggering an imaging operation in the target optimized imaging mode to generate an optimized image, the target optimized imaging mode including a first optimized imaging mode or a second optimized imaging mode, wherein the first optimized imaging mode may be configured to perform an operation for adjusting a transmission parameter of a transmission covering the global imaging region, the second optimized imaging mode may be configured to perform a synchronous operation of an enhanced imaging covering the region of interest and a non-enhanced imaging covering the global imaging region.

Another aspect of the present disclosure provides a system for ultrasonic imaging. The system may include at least one storage device including a set of instructions and at least one processor configured to communicate with the at least one storage device. When executing the set of instructions, the at least one processor is configured to direct the system to perform following operations. The operations may include determining a hybrid wave imaging mode according to information related to one or more imaging demands. The operations may also include obtaining an imaging result by performing a hybrid wave imaging operation according to the hybrid wave imaging mode, wherein the one or more imaging demands may at least include a demand related to an image quality and/or a frame rate, the hybrid wave imaging operation may at least include utilizing a hybrid wave with at least two transmission beam types and/or at least two transmission frequencies for imaging, and the transmission beam types may at least include a focused wave and/or an unfocused wave.

Another aspect of the present disclosure provides a system for ultrasonic imaging. The system may include a hybrid wave imaging mode determination module and a hybrid wave imaging operation module. The hybrid wave imaging mode determination module may be configured to determine a hybrid wave imaging mode according to information related to one or more imaging demands. The hybrid wave imaging operation module may be configured to obtain an imaging result by performing a hybrid wave imaging operation according to the hybrid wave imaging mode, wherein the one or more imaging demands may at least include a demand related to an image quality and/or a frame rate, the hybrid wave imaging operation may at least include utilizing a hybrid wave with at least two transmission beam types and/or at least two transmission frequencies for imaging, and the transmission beam types may at least include a focused wave and/or an unfocused wave.

Another aspect of the present disclosure provides a non-transitory computer readable medium. The non-transitory computer readable medium may include executable instructions that, when executed by at least one processor, direct the at least one processor to perform a method. The method may include determining a hybrid wave imaging mode according to information related to one or more imaging demands. The method may also include obtaining an imaging result by performing a hybrid wave imaging operation according to the hybrid wave imaging mode, wherein the one or more imaging demands may at least include a demand related to an image quality and/or a frame rate, the hybrid wave imaging operation may at least include utilizing a hybrid wave with at least two transmission beam types and/or at least two transmission frequencies for imaging, and the transmission beam types may at least include a focused wave and/or an unfocused wave.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary application scenario of an ultrasonic imaging system according to some embodiments of the present disclosure;

FIG. 2 is a schematic diagram illustrating an exemplary application scenario of an ultrasonic imaging device according to some embodiments of the present disclosure;

FIG. 3 is a flowchart illustrating an exemplary ultrasonic imaging process according to some embodiments of the present disclosure;

FIG. 4 is a flowchart illustrating an exemplary process of determining a target imaging mode according to some embodiments of the present disclosure;

FIG. 5 is a flowchart illustrating an exemplary process of selecting a target imaging mode according to some embodiments of the present disclosure;

FIG. 6 is a flowchart illustrating an exemplary process of obtaining a target imaging result according to some embodiments of the present disclosure;

FIG. 7 is a block diagram illustrating an exemplary processing device according to some embodiments of the present disclosure;

FIG. 8 is a flowchart illustrating an exemplary ultrasonic imaging process according to some embodiments of the present disclosure;

FIG. 9 is a flowchart illustrating an exemplary ultrasonic imaging process according to some embodiments of the present disclosure;

FIGS. 10a-10c are schematic diagrams illustrating determination of a region of interest by a touch screen and/or non-touch screen operation instruction according to some embodiments of the present disclosure;

FIG. 11 is a flowchart illustrating an exemplary process of determining imaging condition data according to a region of interest according to some embodiments of the present disclosure;

FIGS. 12a-12b are schematic diagrams illustrating exemplary focal points required in transmission to cover a global imaging region according to some embodiments of the present disclosure;

FIGS. 13a-13b are schematic diagrams illustrating exemplary focal points required in transmission to cover an effective imaging region according to some embodiments of the present disclosure;

FIG. 14 is a flowchart illustrating an exemplary process of determining a target optimized imaging mode based on the imaging condition data and triggering an imaging operation in the target optimized imaging mode according to some embodiments of the present disclosure;

FIGS. 15a-15b are schematic diagrams illustrating adjusting a transmission parameter according to some embodiments of the present disclosure;

FIGS. 16a-16c are schematic diagrams illustrating adjusting a transmission parameter according to some embodiments of the present disclosure;

FIGS. 17a-17d are schematic diagrams illustrating adjusting a transmission parameter according to some embodiments of the present disclosure;

FIG. 18 is a schematic diagram illustrating an exemplary enhanced imaging operation covering a region of interest according to some embodiments of the present disclosure;

FIG. 19 is a schematic diagram illustrating an exemplary image compounding operation of an enhanced image and a global image according to some embodiments of the present disclosure;

FIGS. 20a-20c are schematic diagrams illustrating an exemplary operating node of an image compounding operation of an enhanced image and a global image according to some embodiments of the present disclosure;

FIG. 21 is a schematic diagram illustrating an optimized imaging setting interface according to some embodiments of the present disclosure;

FIG. 22 is a block diagram illustrating an exemplary processing device according to some embodiments of the present disclosure;

FIG. 23 is a flowchart illustrating an exemplary ultrasonic imaging process according to some embodiments of the present disclosure;

FIG. 24 is a flowchart illustrating an exemplary hybrid wave imaging operation in a hybrid wave imaging mode according to some embodiments of the present disclosure;

FIG. 25 is a schematic diagram illustrating determining a limiting deflection angle and a limiting delay time according to an array element directional restriction condition according to some embodiments of the present disclosure;

FIG. 26 is a schematic diagram illustrating determining a limiting deflection angle and a limiting delay time according to an array element directional restriction condition according to some embodiments of the present disclosure;

FIGS. 27a-27d are schematic diagrams illustrating an exemplary deflection scanning using divergent wave beams according to some embodiments of the present disclosure;

FIG. 28 is a schematic diagram illustrating determining a limiting deflection angle and a limiting delay time according to an array element directional restriction condition according to some embodiments of the present disclosure;

FIG. 29 is a schematic diagram illustrating an exemplary focal point distribution in a hybrid wave imaging mode according to some embodiments of the present disclosure;

FIG. 30 is a schematic diagram illustrating an exemplary transmission imaging of a first hybrid wave imaging mode according to some embodiments of the present disclosure;

FIG. 31 is a schematic diagram illustrating an exemplary focal point distribution in a hybrid wave imaging mode according to some embodiments of the present disclosure;

FIG. 32 is a schematic diagram illustrating an exemplary transmission imaging of a second hybrid wave imaging mode according to some embodiments of the present disclosure;

FIGS. 33a-33c are schematic diagrams illustrating an exemplary acoustic pressure distribution of ultrasonic beams in a hybrid imaging mode according to some embodiments of the present disclosure; and

FIG. 34 is a block diagram illustrating an exemplary processing device according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

The terminology used herein is to describe particular example embodiments only and is not intended to be limiting. As used herein, the singular forms “a,” “an,” and “the” may be intended to include the plural forms as well, unless the context indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

A flowchart is used in the present application to illustrate the operation performed by the system according to the embodiment of the present application. It should be understood that the previous or subsequent operations are not necessarily performed accurately in order. Instead, the steps may be processed in reverse order or simultaneously. These steps may be added to or removed from another procedure.

An effect of ultrasonic imaging may be related to a plurality of factors, including but not limited to an imaging quality of an imaging region, a frame rate, or the like. In the ultrasonic imaging, the imaging quality and the frame rate may influence each other. For example, an image signal-to-noise ratio, which may measure the imaging quality, may be inversely proportional to the pulse repetition frequency of a transmission waveform. As another example, by increasing a count of transmission focal points or imaging line density, the imaging quality indicators such as image space resolution and/or image uniformity may be improved and the frame rate may be reduced at the same time. When a user (such as a doctor or other professional ultrasonic staff) performs the ultrasonic imaging in different scenes or for different objects, or when different manufacturers improve and test an ultrasonic device, the specific adjustment methods for the imaging quality or the frame rate may be different. Therefore, it is necessary to comprehensively consider factors such as the imaging quality or the frame rate for the ultrasonic imaging.

According to some embodiments of the present disclosure, specific imaging operation may be associated with one or more imaging demands of the user, so that the corresponding imaging operation may be performed according to the one or more imaging demands of the user in a plurality of scenarios. For example, a corresponding target imaging mode may be determined based on information related to one or more imaging demands, a corresponding imaging operation may be performed according to the determined target imaging mode to obtain an imaging result that matches the one or more imaging demands, thereby improving comprehensive imaging efficiency and improving user experiences.

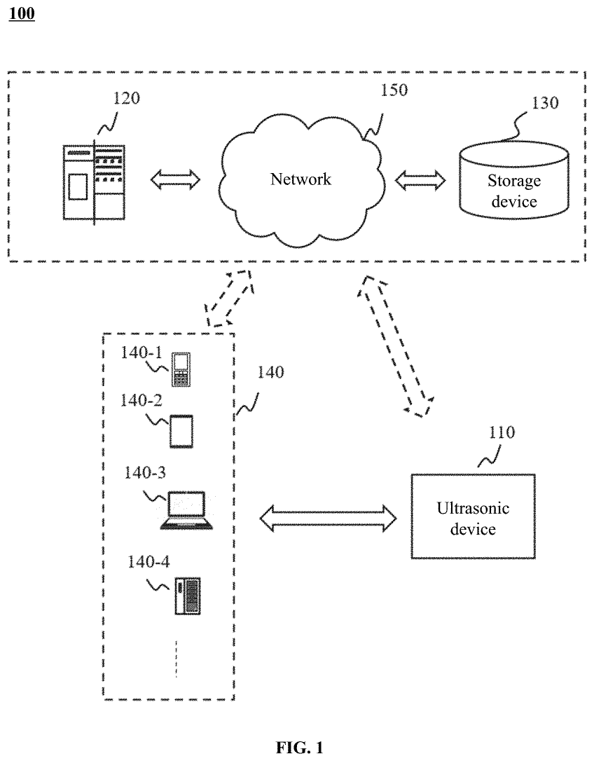

FIG. 1 is a schematic diagram illustrating an exemplary application scenario of an ultrasonic imaging system according to some embodiments of the present disclosure.

As shown in FIG. 1, the application scenario may include an ultrasonic device 110, a processing device 120, a storage device 13, a terminal 140, and/or network 150.

The ultrasonic device 110 may be used to scan an object for diagnostic imaging. The ultrasonic device 110 may be used to view image information of an internal body tissue of the object to assist a doctor for diagnosing a disease. The ultrasonic device 110 may transmit acoustic wave(s) with a relatively high frequency (e.g., ultrasonic wave(s)) to the object through a probe to generate an ultrasonic image. In some embodiments, the object may include a biological object and/or a non-biological object. For example, the object may include a particular part of a human body, such as a neck, a chest, an abdomen, etc., or a combination thereof. As another example, the object may be a patient to be scanned by the ultrasonic device 110. In some embodiments, the ultrasonic image may include at least one of a luminance mode (e.g., a B mode) image, a color mode (e.g., a C mode) image, a motion mode (e.g., an M mode) image, a doppler mode (e.g., a D mode) image, and an elastography mode (e.g., an E mode) image. In some embodiments, the ultrasonic image may include a two-dimensional (2D) image or a three-dimensional (3D) image.

The ultrasonic device 110 may be used for functions such as data acquisition, processing, output, positioning, or the like. The ultrasonic device 110 may include one or more sub-functional devices (e.g., a single sensor device or a sensor system device composed of multiple sensor devices). In some embodiments, the ultrasonic device 110 may include, but is not limited to, an ultrasonic transmission unit (e.g., including an ultrasonic transducer, etc.), an ultrasonic imaging unit, a radio frequency sensing unit, an NFC communication unit, an image acquisition unit, an image display unit, an audio output unit, or any combination thereof. Exemplarily, the ultrasonic imaging unit may be used to process a received signal, including data processing operations in the ultrasonic imaging process such as filtering, demodulation, beamforming, and other data processing. Exemplarily, the image display unit may be used to optimize the display of an image. Exemplarily, the ultrasonic device 110 may obtain imaging object information and/or receive imaging operation instruction information through an information input module (not shown in FIG. 1), for example, a preset demand parameter selection instruction, a user instruction for inputting information related to the one or more imaging demands, or the like. Exemplarily, the ultrasonic device 110 may receive the imaging object information and/or the imaging operation instruction information transmitted from the terminal 140 or the processing device 120 via the network 150, for example, the preset demand parameter selection instruction, the user instruction for inputting the information related to the one or more imaging demands, etc., or transmit intermediate imaging result data or a target image to the processing device 120, the storage device 130, or the terminal 140.

The processing device 120 may include a single server, or a group of servers. The group of servers may be centralized or distributed (e.g., the processing device 120 may be a distributed system). In some embodiments, the processing device 120 may be local or remote. For example, the processing device 120 may access information and/or data stored in the ultrasonic device 110, the terminal 140, and/or the storage device 130 via the network 150. As another example, the processing device 120 may be directly connected to the ultrasonic device 110, the terminal 140, and/or the storage device 130 to access stored information and/or data. In some embodiments, the processing device 120 may be implemented on a cloud platform. For example, the cloud platform may include a private cloud, a public cloud, a hybrid cloud, a community cloud, a distributed cloud, an internal cloud, a multi-layer cloud, etc., or any combination thereof. In some embodiments, the processing device 120 may be implemented on a computing device that includes one or more components.

In some embodiments, the processing device 120 may process information and/or data related to the one or more imaging demands to perform one or more functions described herein. For example, the processing device 120 may determine a target imaging mode based on the information related to the one or more imaging demands. In some embodiments, the processing device 120 may include a processing device 700, a processing device 2200, and/or a processing device 3400. The processing device 700, the processing device 2200, and/or the processing device 3400 may be configured as one or more processing devices. For example, the functions of two or more of the processing device 700, the processing device 2200, and the processing device 3400 may be implemented on a same processing device. As another example, a function of the processing device 700, the processing device 2200, and/or the processing device 3400 may be implemented on multiple processing devices.

In some embodiments, the processing device 120 may include one or more processing engines (e.g., a single-core processing engine or a multi-core processing engine). The processing device 120 may include a central processing unit (CPU), an application specific integrated circuit (ASIC), an application specific instruction set processor (ASIP), a graphics processing unit (GPU), a physical processing unit (PPU), a digital signal processor (DSP), a field programmable gate array (FPGA), a programmable logic device (PLD), a controller, a microcontroller unit, a reduced instruction set computer (RISC), a microprocessor, etc. or any combination thereof. In some embodiments, the processing device 120 may be integrated into the ultrasonic device 110 and/or the terminal 140.

In some embodiments, the ultrasonic device 110, the terminal 140, and/or other possible system components may include the processing device 120, for example, the processing device 120 or a module capable of realizing the functions of the processing device 120 may be integrated into the ultrasonic device 110, the terminal 140 and/or the other possible system components.

In some embodiments, one or more components of the ultrasonic imaging system 100 may transmit data to other components of the ultrasonic imaging system 100 via the network 150. For example, the processing device 120 may obtain information and/or data from the terminal 140, the ultrasonic device 110 and/or the storage device 130 via the network 150, or may transmit the information and/or data to the terminal 140, the ultrasonic device 110 and/or the storage device 130 via the network.

The storage device 130 may be used to store data and/or instructions, wherein the data may refer to a digital representation of information and may include various types of data, such as binary data, textual data, image data, video data, or the like. The instructions may refer to programs that control a device or a device to perform specific functions. For example, the storage device 130 may store one or more preset demand parameters, information related to the one or more imaging demands, target imaging mode, target imaging mode control program, target imaging result, imaging condition data (such as a first condition, etc.), transmission parameter data (such as a first transmission parameter, etc.), hybrid wave echo data (such as first hybrid wave echo data, etc.), a user inputting instruction (such as a touch screen operation instruction, etc.) and/or a preset machine learning algorithm, etc. Various types of data and/or procedures that may be involved in the ultrasonic imaging process.

The storage device 130 may include one or more storage components, and the each storage component may be an independent device or a part of other devices. In some embodiments, the storage device 130 may include a random access memory (RAM), a read only memory (ROM), a mass storage, a removable memory, a volatile read-write memory, or any combination thereof. For example, the mass storage may include a magnetic disk, an optical disk, a solid state disk, or the like. In some embodiments, the storage device 130 may be implemented on a cloud platform. For example, the cloud platform may include a private cloud, a public cloud, a hybrid cloud, a community cloud, a distributed cloud, an internal cloud, a multi-layer cloud, etc., or any combination thereof.

The terminal 140 may refer to one or more terminal devices or software used by the user. The terminal 140 may include a processing unit, a display unit, an input/output unit, a sensing unit, a storage unit, or the like. The sensing unit may include but is not limited to, a light sensor, a distance sensor, an acceleration sensor, a gyroscope sensor, an acoustic detector, etc., or any combination thereof.

In some embodiments, the terminal 140 may be one or any combination of a mobile device 140-1, a tablet computer 140-2, a laptop computer 140-3, a desktop computer 140-4, or other devices with input and/or output functions. In some embodiments, one or more users may use the terminal 140, which may include users who directly use the service, such as ultrasonic diagnostic doctors or ultrasonic testing staff, etc., and may also include other related users, such as hospital medical system end users, etc.

The above examples are only used to illustrate the breadth of the range of terminal 140 and not to limit the scope thereof.

The network 150 may connect components of the system and/or connect the system with external resource portions. The network 150 may communicate between the various components and with other components outside the system, and facilitate the exchange of data and/or information. In some embodiments, the network 150 may be any one or more of a wired network or a wireless network. For example, the network 150 may include a cable network, a fiber optic network, a telecommunications network, an Internet, a local area network (LAN), a wide area network (WAN), a wireless local area network (WLAN), a metropolitan area network (MAN), a public switched telephone network (PSTN), a Bluetooth network, a ZigBee network (ZigBee), a near field communication (NFC), an in-device bus, an in-device line, a cable connection, etc., or any combination thereof. The network connection between the various parts may be in the above-mentioned one way, and may also be in a variety of ways. In some embodiments, the network may be in point-to-point, shared, centralized, etc. various topologies or a combination of multiple topologies. In some embodiments, the network 150 may include one or more network access points. For example, the network 150 may include wired or wireless network access points, such as base stations and/or network exchange points, through which one or more components to and from the ultrasonic imaging system 100 may connect to the network 150 to exchange data and/or information.

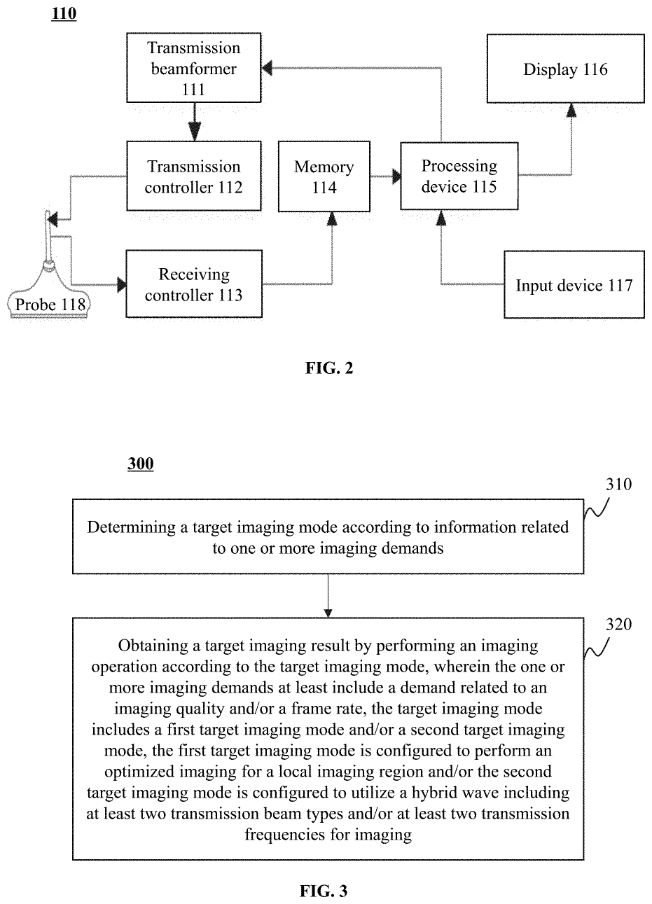

FIG. 2 is a schematic diagram illustrating an exemplary application scenario of an ultrasonic imaging device according to some embodiments of the present disclosure.

The ultrasonic device 110 may include a transmission beamformer 111, a transmission controller 112, a receiving controller 113, a memory 114, a processing device 115, a display 116, an input device 117, and/or a probe 118.

The input device 117 may include but is not limited to devices such as a keyboard, a mice, a tablet, and a touchscreen, and may further control the system through voice and gesture as inputting instructions.

In some embodiments, when user instructions related to the one or more imaging demands are transmitted to the processing device 115 through the input device 117, the processing device 115 may parse the instructions and determine a corresponding target imaging mode, and deliver the corresponding parameter demands of the target imaging mode to the transmission beamformer 111. The transmission beamformer 111 may calculate transmission parameters such as delay time, deflection angle, and/or corresponding parameters of array element according to the parameter demands, and the transmission controller 112 may form a corresponding scan sequence of the target imaging mode according to the transmission parameters and generate a driving voltage signal to excite a transducer array element in the probe 118 to vibrate to form a corresponding ultrasonic wave.

A reflected signal may be produced if an acoustic wave encounters an uneven interface when propagating in medium, the transducer array element of the probe 118 may receive a vibration wave reflected by the medium, the receiving controller 113 may convert a mechanical wave into an electrical signal and/or stored in the memory 114, the processing device 115 may convert the electrical signal into an image and/or output the image to the display 116 according to the operation mode corresponding to the corresponding imaging mode and/or a processing option or adjustment input or selected by the user in real time.

The transmission beamformer 111 may be not limited to hardware circuits, such as an FPGA (programmable gate array) and a DSP (digital signal processing), but may also be a digital beamformer. The transducer array element for transmitting the ultrasonic wave may be a one-dimensional array or a multi-dimensional array set according to the corresponding target imaging mode (such as a first hybrid wave imaging mode or a second hybrid wave imaging mode in the second target imaging mode). In some target imaging modes, different beams or waves with different beam types and/or different transmission parameters (such as a transmission frequency) may not be transmitted at the same time, and one type of wave may be transmitted at a time and complete the reception of the echo before the next transmission, which causes the interference of the echo signals. The use of multi-dimensional array (such as a two-dimensional array) may make use of the array element related parameters such as different widths of multi-array elements to make different array elements suitable for corresponding beams to meet the multi-imaging demands scenes and improve the overall imaging efficiency.

It should be understood that the system and the modules of the system shown in FIG. 1 may be implemented in various ways. For example, in some embodiments, the system and the modules of the system may be implemented by hardware, software, or a combination of software and hardware. The hardware part may be realized by special logic. The software part may be stored in memory and executed by an appropriate instruction execution system, such as a microprocessor or special design hardware. Those skilled in the art may understand that the above methods and systems may be implemented using computer-executable instructions and/or included in processor control code, such as providing such code on carrier media such as magnetic disk, CD or DVD-ROM, programmable memory such as read-only memory (Firmware), or data carrier such as an optical or electronic signal carrier. The system and the modules of the system of the present disclosure may be realized not only by hardware circuits such as VLSI or gate array, semiconductors such as logic chips and transistors, or programmable hardware devices such as field programmable gate array and programmable logic devices but also by software executed by various types of processors, it may also be realized by a combination of the above hardware circuit and software (e.g., a firmware).

It should be noted that the descriptions of the ultrasonic imaging system 100 in FIG. 1 and the ultrasonic device 110 in FIG. 2 are merely provided for the purposes of illustration, and the present disclosure may not be limited to the scope of the embodiments. It may be understood that after understanding the principle of the system, those skilled in the art may arbitrarily combine two or more modules or form a subsystem to connect with other modules without departing from this principle.

In some embodiments, an imaging mode determination module 710, an imaging operation module 720 disclosed in FIG. 7, and/or a region of interest determination module 2210, an imaging condition data determination module 2220, an optimized image generation module 2230 disclosed in FIG. 22, and/or a hybrid wave imaging mode determination module 3410 and a hybrid wave imaging operation module 3420 disclosed in FIG. 34 may be implemented in the ultrasonic device 110 and/or the processing device 120. In some embodiments, the region of interest determination module 2210 and the imaging condition data determination module 2220 may be sub-modules of the imaging mode determination module 710. In some embodiments, the optimized image generation module 2230 may be a sub-module of the imaging operation module 720. In some embodiments, the hybrid wave imaging mode determination module 3410 may be a sub-module of the imaging mode determination module 710, and the hybrid wave imaging operation module 3420 may be a sub-module of the imaging operation module 720.

In some embodiments, the imaging mode determination module 710, the imaging operation module 720 disclosed in FIG. 7, and/or the region of interest determination module 2210, the imaging condition data determination module 2220, the optimized image generation module 2230 disclosed in FIG. 22, and/or the hybrid wave imaging mode determination module 3410 and the hybrid wave imaging operation module 3420 disclosed in FIG. 34 may be different modules in one system, or there may be a module to realize the functions of the above two or more modules. For example, each module may share a storage module, and each module may also have its own storage module. Such deformation is within the protection scope of the present disclosure.

FIG. 3 is a flowchart illustrating an exemplary ultrasonic imaging process according to some embodiments of the present disclosure. In some embodiments, the ultrasonic imaging process 300 may be performed by the processing device 700, the ultrasonic device 110, and/or the processing device 120. In some embodiments, the process 300 may be stored in a storage device (such as the storage device 130) in the form of a program or instruction, and the process 300 may be implemented when the ultrasonic imaging system 100 (such as the processing device 120) executes the program or instruction. In some embodiments, the process 300 may be performed by one or more modules in FIG. 7.

As shown in FIG. 3, the ultrasonic imaging process 300 may include one or more of the following operations.

In 310, a target imaging mode may be determined according to information related to the one or more imaging demands. In some embodiments, operation 310 may be performed by the imaging mode determination module 710.

In 320, a target imaging result may be obtained by performing an imaging operation according to the target imaging mode. In some embodiments, operation 320 may be performed by the imaging operation module 720.

In some embodiments, the one or more imaging demands may include at least a demand related to the imaging quality and/or the frame rate. In some embodiments, the target imaging mode may include a first target imaging mode and/or a second target imaging mode. In some embodiments, the first target imaging mode may be configured to perform an optimized imaging for a local imaging region. In some embodiments, the second target imaging mode may be configured to utilize a hybrid wave including at least two transmission beam types and/or at least two transmission frequencies for imaging.

The one or more imaging demands may refer to corresponding imaging demands of different users (such as an ultrasonic testing staff, an ultrasonic diagnostic doctor, etc.) in a certain imaging scene. The information related to the one or more imaging demands may refer to any information that may reflect or indicate the one or more imaging demands, such as image parameter information. In some embodiments, the one or more imaging demands may include the demand related to the imaging quality and/or the frame rate. In some embodiments, the information related to the one or more imaging demands may include a space resolution, a contrast resolution, a time resolution, an image signal-to-noise ratio, a frame rate, number of frames, an imaging speed, an imaging time, or any other feasible index (or imaging demand parameter) that may reflect the demand related to the imaging quality and/or the frame rate. In some embodiments, the imaging scene may include scenes with different imaging demands in terms of the imaging quality and/or the frame rate, such as an imaging diagnosis scene with high imaging quality demands for pathological tissue area such as a tumor, a cardiac imaging scene with high frame rate demand, a vascular detection scanning imaging scene, etc.

In some embodiments, the one or more imaging demands may be characterized by a corresponding imaging demand level or a specific value. For example, it may be set to “a first imaging quality level”, “a second imaging quality level”, “a third imaging quality level”, etc., which is from high to low according to the imaging quality. As another example, according to the frame rate, it may be set to “a frame rate level 1” and “a frame rate level 2”. As a further example, the imaging quality or the frame rate may be characterized by a specific image signal-to-noise ratio value or an imaging time value, etc.

In some embodiments, the operation 310 may be executed by the following processes: the information related to the one or more imaging demands may be obtained according to the one or more preset demand parameters and/or a user transmission instruction; a target imaging mode may be determined from a first target imaging mode and/or a second target imaging mode according to the information related to the one or more imaging demands.

In some embodiments, the present demand parameter may include an imaging quality parameter and/or a frame rate parameter. In some embodiments, the imaging quality parameter may include a space resolution, a contrast resolution, a temporal resolution, an image signal-to-noise ratio, and/or any other feasible corresponding index parameter that may reflect the imaging quality. In some embodiments, the frame rate parameter may include a frame rate, number or count of frames, an imaging speed, imaging time, and/or any other feasible index parameter that may reflect the frame rate.

In some embodiments, the one or more preset demand parameters may be pre-stored in the ultrasonic device 110, the processing device 120, the storage device 130, and/or the terminal 140. In some embodiments, the one or more preset demand parameters may be obtained in real time from the ultrasonic device 110, the processing device 120, the storage device 130, and/or the terminal 140. In some embodiments, the one or more preset demand parameters may be selected by the user in the form of multiple demand parameter options. For example, on a touch interface of the display 116 of the ultrasonic device 110, an imaging quality parameter menu may have multiple options such as a space resolution, a contrast resolution, a time resolution, and an image signal-to-noise ratio. A frame rate parameter menu may have multiple options such as the frame rate, the number or count of frames, the imaging speed, and the imaging time.

In some embodiments, the information related to the one or more imaging demands may be obtained according to user inputting instructions. In some embodiments, the user inputting instructions may be key inputting instructions, mouse inputting instructions, text inputting instructions, voice instructions, touch screen instructions, gesture instructions, electroencephalogram inputting instructions, eye movement inputting instructions, or any other feasible inputting instruction data. In some embodiments, the user inputting instructions may include a variety of information content related to the demand of the imaging quality and/or the frame rate, for example, the user may select “a first class” or “a third class” imaging demand as the demand of an imaging quality level through a touch screen instruction. As another example, the user may input the imaging demand information (e.g., “a first frame rate level”, and/or “a second imaging quality level”) through the voice instruction. As a further example, the user may input the image signal-to-noise ratio value and/or the specific required value of the frame rate through the text inputting instruction. In some embodiments, the user may input information such as an imaging site and/or an imaging purpose, and the ultrasonic imaging system 100 may automatically match the corresponding information related to the one or more imaging demands through information such as the imaging site and/or the imaging purpose. For example, the user may select a heart as the imaging site, and the ultrasonic imaging system 100 may automatically match the imaging demand information of “the first frame rate level”.

In some embodiments, the information related to the one or more imaging demands included in the instruction(s) may be automatically identified according to the user inputting instruction(s). In some embodiments, the one or more preset demand parameters may be selected according to the identification of corresponding information of the user inputting instruction(s). In some embodiments, the one or more preset demand parameters may be updated according to the user inputting instruction(s) recorded by the ultrasonic imaging system 100.

In some embodiments, the information related to the one or more imaging demands may be obtained by setting or adjusting the corresponding imaging demand parameter in the ultrasonic imaging system 100. The imaging demand parameter may be a characterized data parameter of the information related to imaging demands, i.e., the imaging demand parameter may include all the information related to the one or more imaging demands. In some embodiments, the setting and adjustment of the imaging demand parameters may be performed on the imaging demand parameter setting interface of the ultrasonic imaging system 100. In some embodiments, the imaging demand parameter may include imaging quality indicator data and/or frame rate indicator data. In some embodiments, the information related to one or more imaging demands may be obtained by manually inputting demand information, for example, by receiving an imaging demand adjustment instruction of the user in real time.

In some embodiments, the information related to the one or more imaging demands may be obtained according to specific imaging region characteristics, for example, when imaging a region of interest (ROI) that is a local imaging region, imaging condition data may be determined according to the region of interest, and the information related to the one or more imaging demands may be determined according to the imaging condition data. In some embodiments, the imaging condition data of the region of interest and the region of non-interest may be the same or different, and accordingly, the information related to the one or more imaging demands of the region of interest and the region of non-interest may also be the same or different. Exemplarily, the imaging demand information may be determined according to a type of the region of interest, and when the type of the region of interest is determined to be a diseased tissue such as a tumor, the demand information of “a first imaging quality level” may be obtained, when the type of the region of interest is determined to be a heart detection area or a blood vessel tissue area such as a fast blood flow rate, the demand information of “a first frame rate level” may be obtained, etc. Exemplarily, the imaging demand information may be determined according to a size of the ROI, and when the effective imaging region covering the region of interest region is small, demand information of “a first imaging quality level of the ROI”, “a first frame rate level of the ROI” and/or “a first imaging quality level of the region of non-interest”, “a first frame rate level of the region of non-interest”, etc. may be determined.

In some embodiments, the information related to the one or more imaging demands may be obtained based on tissue motion information (e.g., characteristics of tissue motion) of the detection object. In some embodiments, the tissue motion information of a detection object (such as a certain organ of a patient, etc.) may be obtained, and imaging condition data (such as a transmission parameter of a hybrid beam) may be determined according to the tissue motion information, the information related to the one or more imaging demands may be determined according to the imaging condition data. In some embodiments, in order to better meet the one or more imaging demands of a specific scene of the user, the imaging quality and/or the frame rate demands may be used together with the tissue motion information of a detected portion of the detection object as basic information for determining a hybrid wave imaging mode, or the specific imaging demand information of the corresponding imaging quality and/or the frame rate (e.g., in a cardiac angiography imaging scene of a specific detection object, the imaging time as a frame rate parameter may be set within a specific threshold range to meet the optimal frame rate in the specific scene) may be determined according to the tissue motion information of the detected portion. In some embodiments, the user specific scene may be an imaging scene with more significant tissue motion information, or a functional tissue imaging scene with a higher frame rate demand, such as a cardiac angiography imaging scene, or a blood vessel detection imaging scene, etc. More descriptions about the imaging condition data and related descriptions may be found elsewhere in the present disclosure (e.g., FIGS. 8-21 and the related descriptions).

In some embodiments, a mapping relationship table between the corresponding tissue motion information of various imaging scenarios and the imaging demand information (i.e., the information related to the one or more imaging demands) may be generated. In some embodiments, an imaging demand information recognition program may be set according to the mapping relationship table, and the corresponding imaging demand information may be obtained according to the imaging demand information recognition program and/or the tissue motion information of a certain scene or multiple scenes.

In some embodiments, the imaging demand information identification program may include a preset machine learning identification model, and the imaging demand information may be calculated by inputting the tissue motion information into the preset machine learning identification model. In some embodiments, the preset machine learning recognition model may be a machine learning classification model obtained through model training. In some embodiments, a training process of the machine learning classification model may include the following operations:

Training sample data of tissue movement information and label data of the imaging demand information may be obtained. The training sample data of the tissue motion information and the label data of the imaging demand information may be inputted into a model to be trained, and calculation result data may be output. A model parameter may be updated according to the calculation result data and the label data of the imaging demand information, and the training may be continued until a desired model (i.e., a machine learning classification model) is obtained.

In some embodiments, the training sample data of the tissue motion information and the label data of the imaging demand information may be obtained from ultrasonic imaging historical record data. For example, the training sample data of the tissue motion information and the label data of the imaging demand information may be obtained from the ultrasonic imaging historical record data accumulated by the ultrasonic device 110 for various imaging scenarios and various detection objects. The machine learning classification model may be obtained by training using the training sample data and label data of the training sample data that may reflect an actual imaging demand of the user. When using the machine learning classification model to obtain the imaging demand information, the accuracy and adaptability may be higher, and the imaging demands of the user may be more satisfied, and thus, the actual imaging demand may be improved, and the user imaging experience may be improved at the same time.

In some embodiments, the target imaging mode may be determined by selecting from the first target imaging mode and/or the second target imaging mode based on the information related to the one or more imaging demands. In some embodiments, the judgment of whether a mode condition is satisfied may be performed according to the acquired information related to the one or more imaging demands. If the mode condition for optimized imaging of the local imaging region of interest is satisfied, the first target imaging mode may be selected; if the mode condition for optimized imaging of the local imaging region of interest is not satisfied, the second target imaging mode may be selected.

The target imaging mode may refer to an imaging mode that meets the one or more imaging demands. In some embodiments, the target imaging mode may include the first target imaging mode and/or the second target imaging mode.

The first target imaging mode may be configured to perform imaging cover the global imaging region, and/or perform corresponding optimized imaging operation according to characteristics of the local imaging region in the global imaging region. The global imaging region may refer to the ultrasonic imaging region with a relatively large range in the ultrasonic image, so that a relatively large range of image information (including one or more ROIs and/or peripheral regions) may be viewed in the ultrasonic detection or diagnosis. For example, when the ultrasonic imaging is performed on a certain diseased tissue or organ, the obtained image may include not only regions of interest for ultrasonic detection or diagnosis but also include related peripheral regions outside these regions of interest. In some embodiments, the global imaging region may be or include a largest imaging region detectable by an ultrasonic probe. In some embodiments, the global imaging region may be or include a user-set imaging region. In some embodiments, the global imaging region may include a local imaging region. The local imaging region may refer to one or more regions of interest in the global imaging region. In some embodiments, enhanced imaging covering the local imaging region within the global imaging region may be performed for acoustic energy compensation. In some embodiments, the local imaging region may include one or more regions of interest, i.e., the regions of interest.

In some embodiments, the first target imaging mode may include at least one of a first optimized imaging mode and a second optimized imaging mode. In some embodiments, the first optimized imaging mode may be used to perform an operation for adjusting a transmission parameter of a transmission covering the global imaging region, the second optimized imaging mode may be configured to utilize the hybrid wave including at least two transmission beam types and/or the at least two transmission frequencies for the imaging.

The transmission parameter of the transmission may refer to a corresponding parameter that may affect a transmission process or imaging effect during ultrasonic imaging. In some embodiments, the transmission parameter of the transmission may include a transmission mode, a transmission aperture parameter, a transmission focal point parameter, a transmission declination, a transmission frequency, a transmission waveform, or a gain (i.e., a gain adjustment), or the like, or a combination thereof. In some embodiments, the transmission mode may include focused transmission, diverging wave transmission, wide beam transmission, plane wave transmission, single-array element transmission, a hybrid transmission of two or more transmission modes, or any other available transmission modes. In some embodiments, the transmission aperture parameter may be transmission aperture position or a combination mode of transmission array elements, distance(s) between transmission apertures, distance(s) between receiving apertures or any other feasible transmission aperture parameter. In some embodiments, the transmission focal point parameters may include a count of transmission focal points, transmission focal point position(s), transmission focal point depth(s), or any other feasible transmission focal point parameter. In some embodiments, the transmission waveform may be a sinusoidal waveform, a shock waveform, an arbitrary-shaped waveform generated by superimposing waveforms with multiple frequencies, or any other feasible transmission waveform.

Exemplarily, in a certain scene with a high demand on the frame rate (such as a cardiac examination), the frame rate may not be greatly sacrificed, which may mean that when the effective imaging region is to be imaged, a count of transmission times that may be increased is limited. In this case, the focal point(s) may be set outside the effective imaging region for imaging. If the effective imaging region is wide, the effective transmission region may be enlarged by enlarging the transmission aperture and/or increasing the transmission declination. If the effective imaging region is in a near-field region (e.g., a region close to the probe), high-frequency acoustic wave(s) may produce satisfactory imaging effect in the near-field by increasing the transmission frequency in the effective imaging region.

More descriptions about operations 300, 310, and 320 may be found in FIGS. 8-21, FIGS. 23-33, and the related descriptions, which may not be repeated here.

FIG. 4 is a flowchart illustrating an exemplary process 400 of determining a target imaging mode according to some embodiments of the present disclosure. In some embodiments, process 400 may be performed by the processing device 700, the ultrasonic device 110, and/or the processing device 120. In some embodiments, the process 400 may be stored in the storage device (e.g., a storage device 130) in the form of programs or instructions, when the ultrasonic imaging system 100 (e.g., the processing device 120) executes the program or instructions, the process 400 may be performed. In some embodiments, the process 400 may be performed by one or more of the modules in FIG. 7, FIG. 22, and/or FIG. 34. In some embodiments, process 310 in FIG. 3 may be performed in accordance with the process 400.

In some embodiments, the process 400 may include one or more of the following operations.

In 311, imaging condition data may be determined according to a local imaging region.

In 312, a target imaging mode may be selected from the first optimized imaging mode and the second optimized imaging mode according to the imaging condition data.

The imaging condition data may refer to data used to determine which optimized imaging mode is suitable for the current local imaging region (e.g., a selected ROI). In some embodiments, the imaging condition data may include corresponding parameter data that may reflect the characteristics of the current local imaging region (e.g., a size of ROI). In some embodiments, the imaging condition data may include an area value of the local imaging region. In some embodiments, the imaging condition data may include a ratio of a local imaging region area to a global imaging region area. In some embodiments, the imaging condition data may include relationship data between the transmission condition data of the effective imaging region of the local imaging region and the transmission condition data of the global imaging region. The effective imaging region may refer to an actual imaging region including the local imaging region, so as to ensure the imaging effect in the local imaging region. In some embodiments, the relationship data between the transmission condition data of the effective imaging region and the transmission condition data of the global imaging region may include a difference, a ratio, or any other possible relationship between the transmission condition data of the effective imaging region and the transmission condition data of the global imaging region.

In some embodiments, the imaging condition data may include a transmission condition parameter for the local imaging region. In some embodiments, the imaging condition data may include a transmission condition parameter for the local imaging region or a transmission condition parameter for the effective imaging region. The transmission condition parameter may be a corresponding condition parameter indicating which optimized imaging mode is satisfied. In some embodiments, the transmission condition parameter may be the count of focal points required for the transmission of the currently selected local imaging region, the count of transmission times required for the transmission of the currently selected local imaging region, or the transmission interval required for the transmission of the currently selected local imaging region (i.e., a time interval from reception of signals in a last transmission to a start of a next transmission), etc. In some embodiments, the count of focal points, the count of transmission times, and the time of transmission required for different partial imaging regions in one or more transmission modes may be determined according to historical record data. For example, according to a preset imaging condition calculation program, the ratio of the count of focal points required for the transmission of the current local imaging region to the count of focal points required for the transmission of the global imaging region may be calculated. The transmission herein may be a focused transmission, a diverging wave transmission, a wide beam transmission, any other possible waveform transmission mode, or a hybrid transmission mode of the transmission modes.

In some embodiments, the imaging condition data may be pre-calculated and stored in an imaging condition data determination module 420, a storage device 130, or an ultrasonic device 110 by the preset imaging condition calculation program according to characteristic data of the historically selected local imaging region. In some embodiments, the imaging condition data may trigger the preset imaging condition calculation program for real-time calculation according to the currently selected local imaging region.

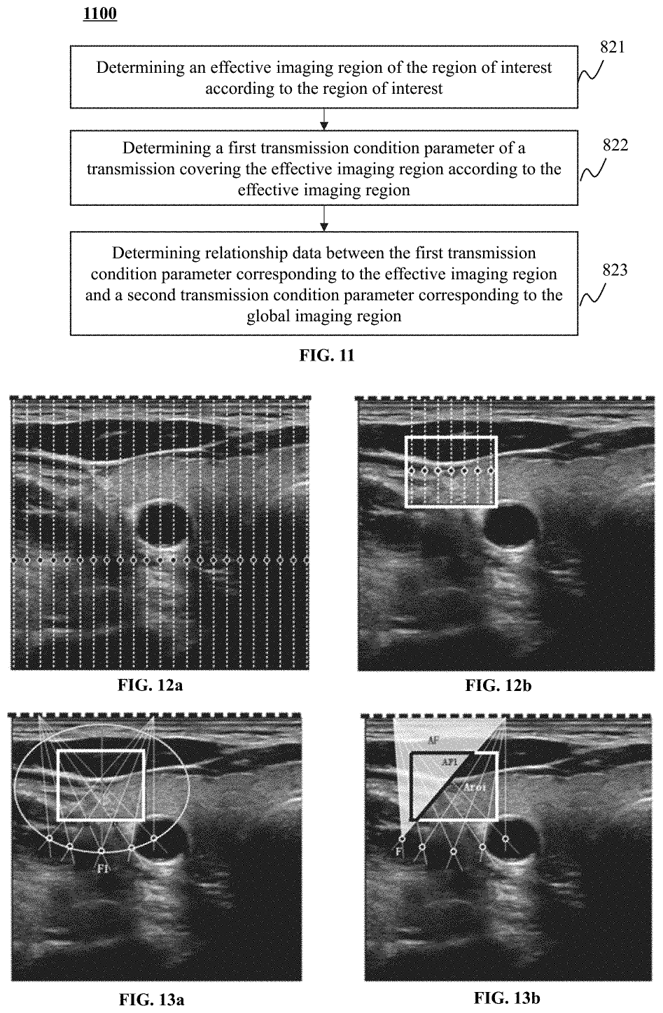

In some embodiments, the imaging condition data may be determined according to the ROI. More description may be found in FIG. 11 and the related descriptions, which may be repeated here.

FIG. 5 is a flowchart illustrating an exemplary process of selecting a target imaging mode according to some embodiments of the present disclosure. In some embodiments, process 500 may be performed by a processing device 700, an ultrasonic device 110, and/or a processing device 120. In some embodiments, the process 500 may be stored in a storage device (such as the storage device 130) in the form of a program or instruction, and the process 500 may be implemented when the ultrasonic imaging system 100 (such as the processing device 120) executes the program or instruction. In some embodiments, the process 500 may be performed by one or more modules in FIG. 7, FIG. 22, and/or FIG. 34. In some embodiments, operation 312 in FIG. 4 may be performed according to the process 500.

In some embodiments, the process 500 may include one or more of the following operations.

In 3121, in response to imaging condition data satisfying a first condition, the first optimized imaging mode may be designated as the target imaging mode.

In 3122, in response to the imaging condition data does not satisfy the first condition, the second optimized imaging mode may be designated as the target imaging mode.

The first condition may refer to a condition that the imaging condition data meets a specific threshold range. In some embodiments, the first condition may be that a ratio of the local imaging region to the global imaging region is greater than a set threshold. In some embodiments, the first condition may be that relationship data between the transmission condition data of the effective imaging region of the local imaging region and the transmission condition data of the global imaging region is less than the set threshold. In some embodiments, the first condition may be that relationship data between transmission condition data of the effective imaging region of the ROI and the transmission condition data of the global imaging region is less than the set threshold, for example, a ratio between required count of transmission of the effective imaging region of the ROI and the required count of focal points of the global imaging region is less than the threshold.

In some embodiments, in response to the relationship data being less than the threshold, an imaging operation in the first optimized imaging mode may be triggered, which may include adjusting the transmission parameter(s) of the transmission while satisfying a frame rate demand to enhance an ultrasonic wave of the region of interest.

In some embodiments, in response to the relationship data being no less than the threshold, an imaging operation in the second optimized imaging mode may be triggered, which may include a synchronous operation of an enhanced imaging covering the region of interest and a non-enhanced imaging covering the global imaging region; and/or performing an image compounding operation on the enhanced image and the global image. In some embodiments, if the relationship data is equal to the threshold, an imaging operation in the first optimized imaging mode or an imaging operation in the second optimized imaging mode may be triggered according to the need.

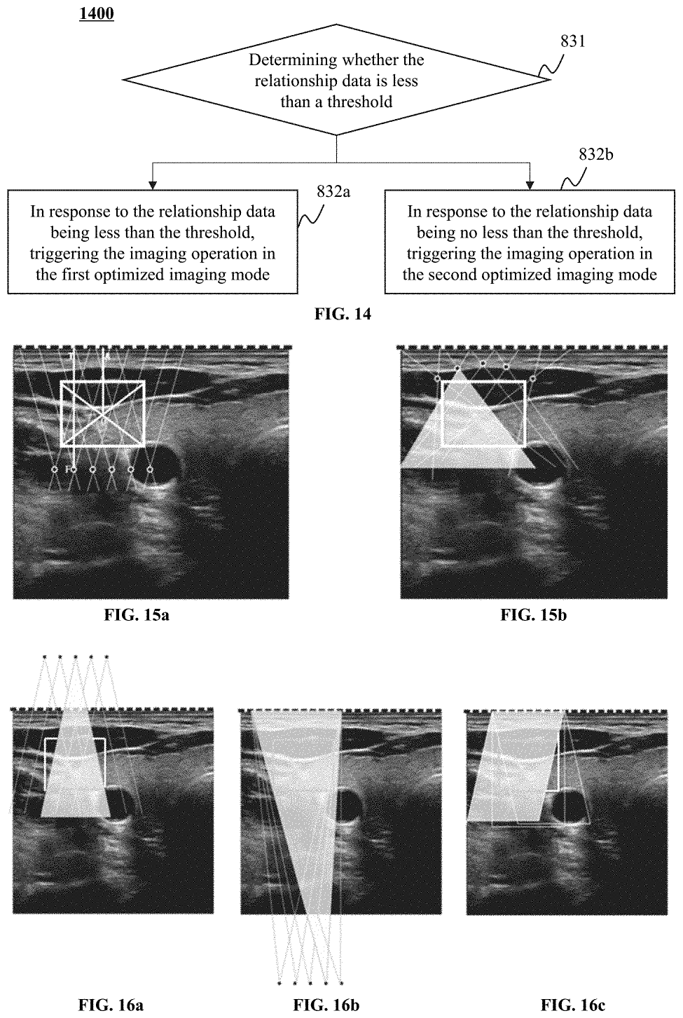

More descriptions about operation 3121 and operation 3122 may be found in FIG. 14 and related descriptions, which may not repeat here.

The second target imaging mode may refer to a hybrid wave imaging mode that uses a hybrid wave with different transmission beam types and/or different transmission frequencies to achieve optimized imaging. The hybrid wave imaging mode may refer to an imaging mode that may be used to perform hybrid imaging using a hybrid wave of different transmission beam types and/or different transmission frequencies on a detection object. In some embodiments, the transmission beam type may include a focused wave type and/or an unfocused wave type, and/or any other feasible beam type. In some embodiments, the unfocused wave may include a plane wave, a divergent wave, a wide beam, or any other feasible unfocused beam type. In some embodiments, the hybrid wave imaging mode may include an imaging mode with the same transmission frequency and different transmission beam types, for example, the divergent wave and the focused wave are both used in a hybrid wave mode with the same transmission frequency (e.g., 7.5 MHz). In some embodiments, the hybrid wave imaging mode may be an imaging mode with different transmission frequencies and the same transmission beam type, for example, a hybrid wave imaging mode using two focused waves or two unfocused waves with transmission frequencies of 7.5 MHz and 5 MHz respectively. In some embodiments, the hybrid wave imaging mode may be an imaging mode with different transmission frequencies and different transmission beam types, for example, a hybrid wave imaging mode of the divergent wave with a transmission frequency of 7.5 MHz, a focused wave with a transmission frequency of 10 MHz, and a plane wave with a transmission frequency of 5 MHz. It should be noted that in the hybrid wave imaging mode, the count of transmission times of the corresponding transmission beams of different transmission beam types and/or different transmission frequencies is not specially limited. For example, in a specific hybrid wave transmission mode, the plane wave with the same transmission frequency may be transmitted multiple times at multiple different time nodes or cycles, or may be transmitted only once or a cycle. As another example, the divergent wave may be transmitted twice or multiple times at different frequencies. As a further example, the divergent wave, the focused wave, and the plane wave with different emission frequencies may be transmitted for imaging once respectively. In addition, it should be noted that in the hybrid wave imaging mode, a sequence of imaging using corresponding beams with different transmission beam types and/or different transmission frequencies is not particularly limited. For example, using the divergent wave first and then using the focusing wave in imaging. As another example, using the divergent wave with a transmission frequency of 7.5 MHz firstly in imaging, then using the focused wave with a transmission frequency of 10 MHz in imaging, and then using the plane wave with a transmission frequency of 5 MHz in imaging, and so on.

In some embodiments, the second target imaging mode may include at least one of a first hybrid wave imaging mode and a second hybrid wave imaging mode. In some embodiments, the first hybrid wave imaging mode may be used for a full aperture hybrid transmission operation, and the second hybrid wave imaging mode may be used for a moving aperture hybrid transmission operation.

In some embodiments, the full aperture hybrid transmission operation may be a transmission operation in which all apertures of the array elements participate in the transmission during the focused wave and/or the unfocused wave transmission to cover a large imaging region, thereby improving the frame rate due to a fast imaging speed, a wide coverage, a uniform acoustic field, and a small count of transmission times of the unfocused wave, and thus, the imaging quality may be improved through the enhancement of the focused wave energy, the expected imaging demands of the user may be met more effectively. In some embodiments, the full aperture hybrid transmission operation may be the unfocused wave transmission in which all apertures participate in the transmission, or the focused wave transmission in which the local aperture (i.e., the partial aperture) may participate in the transmission (e.g., in a specific region or the region of interest that needs a relatively high imaging quality) to allocate resources reasonably, and save costs while meeting the imaging demands of the user.

In some embodiments, the moving aperture hybrid transmission operation may be a transmission operation in which all apertures or local apertures of the array elements participate in the transmission according to a corresponding hybrid transmission order when the focused wave and/or the unfocused wave is transmitted. In some embodiments, the moving aperture hybrid transmission operation may be a transmission operation in which both the focused wave (e.g., the focused wave) and the unfocused wave (e.g., the diverging wave) are transmitted using local apertures according to a corresponding hybrid transmission order. In some embodiments, during the moving aperture hybrid transmission operation, the focused wave and the unfocused wave may be alternately transmitted according to a corresponding hybrid transmission order. In some embodiments, the order, the rule, or the procedure for hybrid transmissions may include individual transmission time node settings and/or alternate transmission time interval settings for the focused wave, the unfocused wave, or the like. The moving aperture hybrid transmission operation may be used to transmit a specific sequence of different hybrid beams set according to a corresponding transmission order or rule, which may make comprehensive use of the respective advantages of multiple beams to scan in important regions (such as the region of interest) to obtain hybrid wave echo data with rich information (such as echo signal data, echo image, or imaging data of multiple beams), and make it convenient for subsequent echo signal recombination or image recombination processing, so as to meet the personalized imaging demands of different users and different scenes.

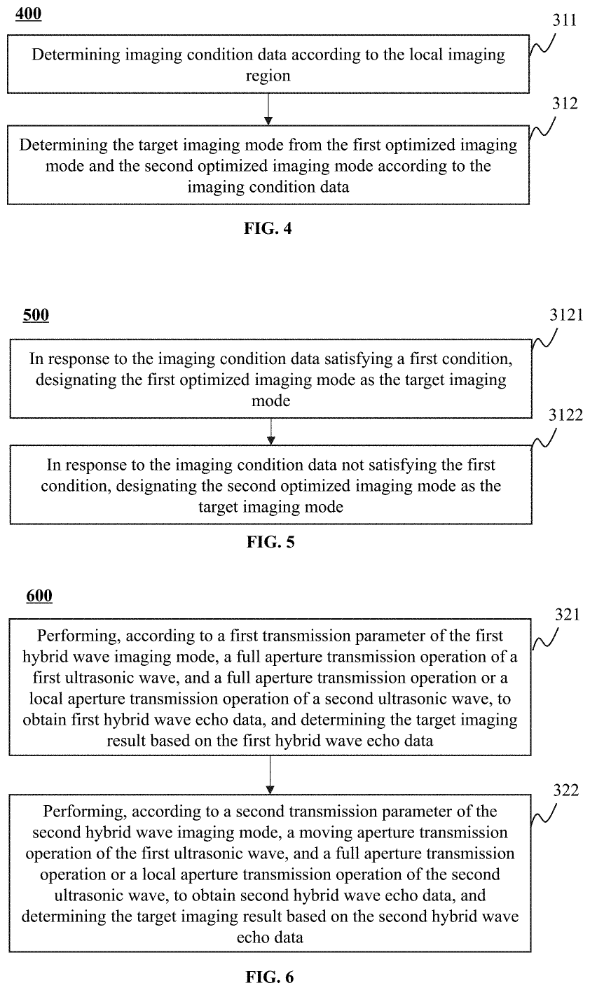

FIG. 6 is a flowchart illustrating an exemplary process of obtaining a target imaging result according to some embodiments of the present disclosure. In some embodiments, process 600 may be performed by a processing device 700, an ultrasonic device 110, and/or a processing device 120. In some embodiments, the process 600 may be stored in a storage device (such as the storage device 130) in the form of a program or instruction, and the process 600 may be implemented when the ultrasonic imaging system 100 (such as the processing device 120) executes the program or instruction. In some embodiments, the process 600 may be performed by one or more modules in FIG. 7, FIG. 22, and/or FIG. 34. In some embodiments, if the target imaging mode is a second target imaging mode, operation 320 in FIG. 3 may be performed according to the process 600.

In some embodiments, the process 600 may include one or more of the following operations.

In 321, according to a first transmission parameter of the first hybrid wave imaging mode, a full aperture transmission operation of a first ultrasonic wave, and a full aperture transmission operation or a local aperture transmission operation of a second ultrasonic wave may be performed, to obtain first hybrid wave echo data, and the target imaging result may be determined based on the first hybrid wave echo data.

In 322, according to a second transmission parameter of the second hybrid wave imaging mode, a moving aperture transmission operation of the first ultrasonic wave, and a full aperture transmission operation or a local aperture transmission operation of the second ultrasonic wave may be performed, to obtain second hybrid wave echo data, and the target imaging result may be determined based on the second hybrid wave echo data.

In some embodiments, triggering hybrid wave imaging operation in a first hybrid wave imaging mode according to the hybrid wave imaging mode includes but is not limited to: determining one or more positions of a first focal point (also referred to as a first focal point position); determining a first transmission parameter of a first hybrid wave imaging mode according to the first focal point position; performing the full aperture transmission operation using a first ultrasonic wave and the full aperture transmission operation or local aperture transmission operation using a second ultrasonic wave according to a first transmission parameter of the first hybrid wave imaging mode to obtain the first hybrid wave echo data. In some embodiments, the first focal point position may satisfy a boundary condition of the first focal point.

In some embodiments, triggering hybrid wave imaging operation in a second hybrid wave imaging mode according to the hybrid wave imaging mode includes but is not limited to: determining one or more positions of a second focal point (also referred to as a second focal point position); determining a second transmission parameter of a second hybrid wave imaging mode according to the second focal point position; performing the moving aperture transmission operation using the first ultrasonic wave and the full aperture transmission operation or the local aperture transmission operation using the second ultrasonic wave according to a second transmission parameter of the second hybrid wave imaging mode to obtain the second hybrid wave echo data. In some embodiments, the second focal point position may satisfy a boundary condition of the second focal point.

More descriptions about operation 321 and operation 322 may be found in FIG. 24 and related descriptions, which may not repeat here.

FIG. 7 is a block diagram illustrating an exemplary processing device according to some embodiments of the present disclosure.

As shown in FIG. 7, the processing device 700 may include an imaging determination module 710 and an imaging operation module 720. The imaging mode determination module 710 may be used to determine a target imaging mode according to information related to the one or more imaging demands. The imaging operation module 720 may be used to obtain the target imaging result by performing the imaging operation according to the target imaging mode. The one or more imaging demands may at least include the demand related to the imaging quality and/or the frame rate. The target imaging mode may include the first target imaging mode and/or the second target imaging mode. The first target imaging mode may be configured to perform an optimized imaging for a local imaging region, and/or, the second target imaging mode may be configured to utilize the hybrid wave with at least two transmission beam types and/or at least two transmission frequencies for imaging. More descriptions about the corresponding functions performed by the imaging mode determination module 710 and the corresponding functions performed by the imaging operation module 720 may be found in FIGS. 1-6 and the related descriptions, which may not be repeated here.

An ultrasonic imaging system provided by some embodiments of the present disclosure may include: at least one storage medium storing a set of instructions; at least one processor in communication with the at least one storage medium, when executing the stored set of instructions, the at least one processor causes the system to: determine a target imaging mode according to information related to the one or more imaging demands; obtain the target imaging result by performing the imaging operation according to the target imaging mode; wherein the one or more imaging demands may at least include a demand related to the imaging quality and/or the frame rate, the target imaging mode may include the first target imaging mode and/or the second target imaging mode, the first target imaging mode may be configured to optimize a local imaging region and/or the second target imaging mode may utilize a hybrid wave with different transmission beam types and/or different transmission frequencies for imaging. More descriptions about the ultrasonic imaging system may be found in FIGS. 1-6, which may not be repeated here.

An ultrasonic imaging system provided by some embodiments of the present disclosure may include a processor, the processor may be used to execute the ultrasonic imaging process described in any of the above embodiments (see the related descriptions of FIGS. 1-6), which may not be repeated here.

A non-transitory computer readable medium including executable instructions, the instructions, when executed by at least one processor, causing the at least one processor to effectuate a method comprising: the ultrasonic imaging process described in any of the above embodiments (see the related descriptions of FIGS. 1-6) may be performed, which may not be repeated here.

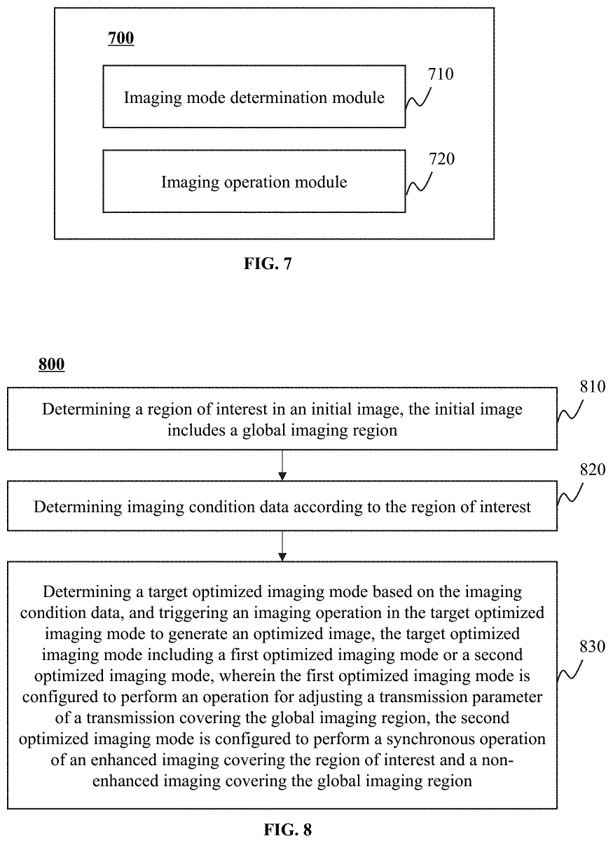

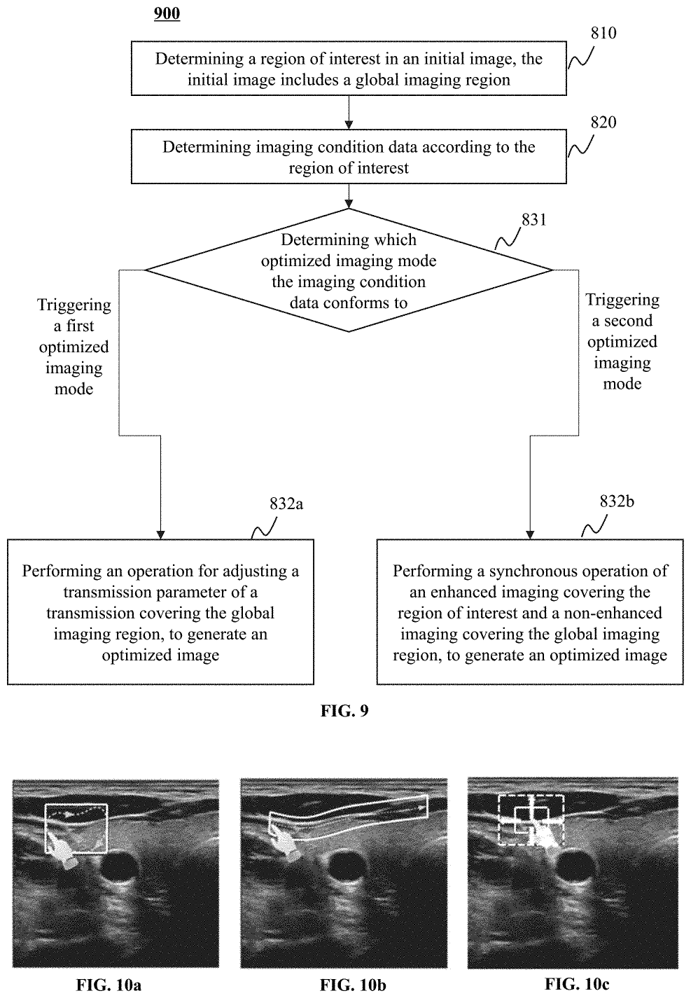

FIG. 8 is a flowchart illustrating an exemplary ultrasonic imaging process 800 according to some embodiments of the present disclosure. FIG. 9 is a flowchart illustrating an exemplary ultrasonic imaging process 900 according to some embodiments of the present disclosure. In some embodiments, the process 800 may be performed by the processing device 2200, the ultrasonic device 110, and/or the processing device 120. In some embodiments, the process 800 may be stored in a storage device (such as the storage device 130) in the form of a program or instruction, and the process 800 may be implemented when the ultrasonic imaging system 100 (such as the processing device 120) executes the program or instruction. In some embodiments, the process 800 may be performed by one or more modules in FIG. 7, FIG. 22, and/or FIG. 34. As shown in FIG. 8 and FIG. 9, in some embodiments, the process 800 or the process 900 may include one or more of the following operations. In some embodiments, the process 800 of FIG. 8 may be implemented in accordance with process 900 of FIG. 9.

In 810, a region of interest may be determined in an initial image. The initial image may include a global imaging region. In some embodiments, operation 810 may be performed by a region of interest determination module 2210.

In 820, imaging condition data may be determined according to the region of interest. In some embodiments, operation 820 may be performed by the imaging condition data determination module 2220.

In 830, a target optimized imaging mode may be determined based on the imaging condition data, and imaging operation in the target optimized imaging mode may be triggered to generate an optimized image. In some embodiments, the optimized imaging mode may at least include the first optimized imaging mode and/or the second optimized imaging mode. The first optimized imaging mode is configured to perform an operation for adjusting the transmission parameter of the transmission covering the global imaging region. The second optimized imaging mode is configured to perform the synchronous operation of an enhanced imaging covering the region of interest and a non-enhanced imaging covering the global imaging region. In some embodiments, operation 830 may be performed by the optimized image generation module 2230.

In some embodiments, operation 830 may be performed by one or more of the following operations.

In 831, the target optimized imaging mode may be determined based on the imaging condition data, and whether to trigger the first optimized imaging mode or the second optimized imaging mode may be determined according to the determination result.

When determining to trigger the first optimized imaging mode, the process may proceed to operation 832a: performing the operation for adjusting the transmission parameter of the transmission covering the global imaging region to generate the corresponding optimized image. When determining to trigger the second optimized imaging mode, the process may proceed to operation 832b: performing the synchronous operation of the enhanced imaging covering the local imaging region and the non-enhanced imaging covering the global imaging region to generate the corresponding optimized image.

By comprehensively considering interrelated factors affecting the overall imaging efficiency, such as the imaging quality, the frame rate, and transmission time, and the one or more imaging demands or diagnosis demands for different imaging conditions, the target optimized imaging mode may be determined based on the imaging condition data corresponding to a selected ROI, and the corresponding optimized imaging mode may be triggered, thereby providing corresponding imaging schemes with high adaptability, improving imaging efficiency, and meeting a variety of imaging demands.

The initial image may refer to an initial image obtained before optimized imaging, wherein the initial image may include a global imaging region.

In some embodiments, the region of interest determination module 2210 may acquire the initial image.

In some embodiments, the initial image may be generated by the ultrasonic device 110. In some embodiments, the ultrasonic device 110 may transmit ultrasonic pulses to a detection object, receive ultrasonic echo signal(s) reflected by the detection object, and output the initial image after processing the received ultrasonic echo signal(s). In some embodiments, the processing may include filtering processing, demodulation processing, beamforming processing, compounding (e.g., line compounding, frequency compounding, space compounding, etc.), envelope processing, logarithmic processing, and image post-processing (e.g., gray processing, speckle noise suppression, edge thinning, etc.). In some embodiments, the initial image may be generated by an initial imaging sub-module (not shown in FIG. 22) of the region of interest determination module 2210.

In some embodiments, the initial image may be obtained from the processing device 120, the storage device 130, or the terminal 140. For example, a pre-generated image may be stored by the storage device 130. As another example, a corresponding initial ultrasonic image may be obtained from a patient user terminal, a user terminal for ultrasonic examination or diagnosis (e.g., a user terminal of a patient storing historical ultrasonic examination results, a user terminal of an ultrasonic examiner, and a user terminal of a doctor).

In some embodiments, the initial image may be obtained from any possible system component in the ultrasonic imaging system 100. For example, a cloud server or a background system. In some embodiments, the initial image may be obtained from a system other than the ultrasonic imaging system 100. For example, from one or more hospital medical systems associated with the ultrasonic imaging system 100. It should be noted that the above acquisition of the initial image is only an example, and the embodiment of this specification is not particularly limited.

In some embodiments, the ROI may be determined using at least one of an artificial intelligence automatic recognition algorithm, an automatic tracking algorithm, and touch-screen and/or non-touch-screen operation instructions, wherein the artificial intelligence automatic recognition algorithm or the automatic tracking algorithm has the advantages of fast speed and high efficiency, the touch-screen and/or non-touch-screen operation instructions may realize a dynamic adjustment in determining the ROI and the user personalized demands for determining the ROI, thereby improving the interactivity. In some embodiments, the ROI may be determined by using one or more combination methods of the artificial intelligence automatic recognition algorithm, the automatic tracking algorithm, and the touch-screen and/or non-touch-screen operation instructions. Specifically, the corresponding settings of multiple combination methods may be made according to the actual needs to comprehensively utilize the advantages of each method, improve the efficiency of determining the ROI, and meet the ROI selection needs of users in a variety of different scenarios.

In some embodiments, the ROI may be any region in the global imaging region. In some embodiments, the ROI may include a plurality of local ROls. In some embodiments, the ROI may have a closed shape (such as a rectangle) or a non-closed shape (such as a line segment). In some embodiments, the closed shape may be a regular shape (such as a square) or an irregular shape.

In some embodiments, the ROI may be determined by automatically identifying the ROI using a preset machine learning algorithm (e.g., a deep learning algorithm). In some embodiments, an initially selected or drawn ROI may be tracked using a preset target detection model. Even the probe changes, it may still realize an automatic tracking of ROI to improve the efficiency of ROI determination. In some embodiments, the preset target detection model may be a Single Shot Multibox Detector (SSD), a Faster Region-Convolutional Neural Network (Faster R-CNN), a You Only Look Once (YOLO), or any other feasible target detection model.

In some embodiments, the ROI may be determined by detecting and/or matching image features (such as feature points, feature lines, feature regions, etc.) using a preset feature algorithm method and/or a preset feature matching algorithm. In some embodiments, the preset feature detection algorithms may include ScaleInvariant Feature Transform (SIFT) operator, Speeded Up Robust Feature (SURF) operator, Features from Accelerated Segment Test (FAST) operator, Local Binary Pattern (LBP) operator, Histogram of Oriented Gradient (HOG), or any other feasible feature detection algorithm. The preset feature matching algorithm may include a Random Sample Consensus (RANSAC) algorithm, an Oriented FAST and Rotated BRIEF (ORB) algorithm, or any other feasible feature detection method.

In some embodiments, a preset template matching algorithm may be used directly to find a region matching the ROI in the initial image, and/or the ROI may be determined by tracking the ROI. In some embodiments, the preset template matching algorithm may be a contour-based template matching algorithm, an edge gradient-based template matching algorithm, or any other feasible template matching algorithm.

In some embodiments, the ROI may be determined through a touch screen and/or non-touch screen operation instruction. In some embodiments, the touch screen and/or non-touch screen operation instruction may include a touch screen drawing operation instruction, a trackball operation instruction, an air gesture drawing operation instruction, and/or any other feasible input operation instructions.

FIGS. 10a-10c are schematic diagrams illustrating determination of a region of interest by the touch screen and/or non-touch screen operation instruction according to some embodiments of the present disclosure. The region enclosed by an outermost boundary line of each image (i.e., an entire image region shown by the initial image) may be regarded as a global imaging region of the initial image.

Exemplarily, as shown in FIG. 10a, when the region of interest of the ultrasonic detection user or the doctor user is a region, the region of interest determination 2210 may receive a touch screen operation instruction (e.g., a manual drawing etc.) input by the ultrasonic detection user or the doctor user, and drawing an end-to-end line in an effective imaging region (as shown by a solid line in the figure) according to the touch screen operation instruction, i.e., a closed shape may be formed and the drawn trajectory line (as shown by a dotted line in the figure) is a boundary of the ROI. Here, the closed shape of the ROI may be any regular shape or irregular shape.

The effective imaging region may refer to an actual imaging region including the ROI to guarantee an imaging effect of the ROI. In some embodiments, the area of the effective imaging region may be equal to the ROI area. In some embodiments, the area of the effective imaging region may be bigger than the ROI area. In some embodiments, a ratio of the area of the effective imaging region to the area of the ROI may range from 1.5 to 2.0 (e.g., 1.8) to avoid incomplete imaging of the edge the ROI, reduce imaging of non-ROI region, prevent redundant calculation, and reduce the amount of calculation. In some embodiments, the effective imaging region may be a closed shape (such as an ellipse) or a non-closed shape (such as a curved line segment). In some embodiments, the closed shape may be a regular shape (e.g., a diamond) or an irregular shape. In some embodiments, the closed shape or the non-closed shape of the effective imaging region may approximate or conform to the closed shape or non-closed shape of the ROI.

Exemplarily, as shown in FIG. 10b, if the region of interest determined by the ultrasonic detection user or the doctor user is an elongated region, the region of interest determination module 2210 may receive a non-touch operation instruction (e.g., an instruction of drawing from a trackball or an air gesture, etc.) input by the ultrasonic detection user or the doctor user, and draw a curved line segment covering the ROI (when it is inconvenient to draw the closed shape) in the effective imaging region (as shown by the solid line in the figure) according to the non-touch operation instruction. In some embodiments, the trackball may be used to mark an ROI key point or directly draw a corresponding shape of the ROI.

Exemplarily, as shown in FIG. 10c, if the ultrasonic detection user or the doctor user adjusts a selected ROI randomly or dynamically, the region of interest determination module 2210 may receive a touch/non-touch operation instruction (e.g., manual touch screen click, trackball click, or non-touch screen default selection instruction, etc.) input by the ultrasonic detection user or the doctor user, an image display and manipulation interface may display a regular shape (e.g., a regular shape such as rectangle, circle, or ellipse, or the like) as shown by the solid line frame in the figure according to a default setting, the shape may be adjusted according to a received touch screen swipe instruction or an air swipe instruction along a direction of the arrow shown in the figure to the region indicated by the dotted box in the figure, and the size of the ROI region may be adjusted by corresponding operation instructions according to demands of the user (such as the ultrasonic detection user, a patient user, a diagnostic user, or a physician user, etc.). For example, when using the trackball to select the ROI, the key may be pressed, a starting point may be determined, the image display and manipulation interface may display a default rectangular region centered on the starting point, the trackball around the rectangular region may be rolled, the drawn region may expand or shrink as the trackball rolls, and the key may be pressed to determine an end point finally, i.e., the ROI selection may be finished.

In some embodiments, the effective imaging region determination manner in the image display and manipulation interface may be similar with the ROI, more description may be found in the manner and the process about the ROI selection, which may not be repeated.

It should be noted that the foregoing determination manner of the ROI or the effective imaging region is only exemplary, and should not be construed as a limitation to the present disclosure, and any feasible manner may be adopted to determine the ROI or the effective imaging region without departing from the inventive concept of the present disclosure.

In 820, the imaging condition data may be determined according to the region of interest.

In some embodiments, the imaging condition data may include corresponding parameter data that may reflect characteristics of a currently selected ROI (such as the size of the ROI region, etc.). In some embodiments, the imaging condition data may include the ratio of the ROI region area to the global imaging region area.

In some embodiments, the imaging condition data may include a transmission condition parameter of the ROI. In some embodiments, the imaging condition data may include a transmission condition parameter of the ROI transmission condition parameter and/or the effective imaging region. The transmission condition parameter may refer to a corresponding condition parameter that indicates which optimal imaging mode is satisfied. In some embodiments, the transmission condition parameter may be the count of focal points required for the examination of the currently selected ROI, the count of transmission times, or the transmission time interval (i.e., a time interval from reception of signals in a last transmission to a start of a next transmission), etc. In some embodiments, the count of focal points, the count of transmission times, or the transmission time interval, etc., required by different ROls in corresponding transmission modes (e.g., a focused transmission mode) may be determined according to historical record data.

In some embodiments, the imaging condition data may include the relationship data between the transmission condition data of the ROI and the transmission condition data of the global imaging region. In some embodiments, the relationship data between the transmission condition data of the ROI and the transmission condition data of the global imaging region may include a difference, a ratio, or any other possible relationship between the transmission condition data of the ROI and the transmission condition data of the global imaging region. For example, a ratio of the count of focal points required for the examination of the currently selected ROI to the count of focal points required for the examination of the global imaging region according to a preset program for determining the imaging condition. The transmission may include focused transmission, diverging wave transmission, broad beam transmission, or any other possible waveform transmission pattern, or a hybrid of different transmission beam types and/or different transmission frequencies.

In some embodiments, the imaging condition data may include the relationship data between the transmission condition data of the effective imaging region of the ROI and the transmission condition data of the global imaging region. In some embodiments, the relationship data between the transmission condition data of the effective imaging region of the ROI and the transmission condition data of the global imaging region may include the difference, the ratio, or any other possible relationship between the transmission condition data of the effective imaging region of ROI and the transmission condition data of the global imaging region.

In some embodiments, the first optimized imaging mode and/or the second optimized imaging mode may include: using a hybrid wave with different transmission beam types and/or different transmission frequencies for imaging operation, and the transmission beam types may at least include a focused wave and/or an unfocused wave. In some embodiments, which transmission beam type(s) and/or which transmission frequency of the hybrid wave may be used for the imaging operation in the first optimized imaging mode or the second optimized imaging mode may be determined according to the imaging condition data. In some embodiments, which transmission beam type(s) and/or which transmission frequency of the hybrid wave may be used for the imaging operation in the first optimized imaging mode or the second optimized imaging mode may be determined according to the relationship data between the transmission condition data of the effective imaging region of the ROI and the transmission condition data of the global imaging region. In some embodiments, the imaging operation using the hybrid wave may include the full aperture hybrid transmission operation and/or the moving aperture hybrid transmission operation.

Exemplarily, if the selected ROI area or the ratio of the selected ROI area to the area of the global imaging region is small, which may mean that the ROI area or a proportion of the ROI in the global imaging region is small, then a synchronous operation of an enhanced imaging covering the ROI using focused wave(s) and a non-enhanced imaging covering the global imaging region using unfocused wave(s) (e.g., a plane wave) may be performed, in which transmission frequencies of the focused wave(s) and the unfocused wave(s) may be equal or not. Exemplarily, if a ratio of the count of focal points required by the selected ROI to the count of focal points required by the global imaging region is big, which may mean that if a certain type of transmission (e.g., using focused wave(s)) is used in the ROI region, the count of transmission times is big or the transmission duration time is big, then the transmission parameter may be adjusted in the global imaging region through the first optimized imaging mode (e.g., by increasing the transmission frequency in the ROI), or alternately, through the second optimized imaging mode, at the same time of the non-enhanced imaging operation using unfocused wave(s) (such as a divergent wave) in the global imaging region, the enhanced imaging operation of the focused wave(s) may be further performed in the ROI.

By combining the hybrid wave imaging in the first optimized imaging mode and/or the second optimized imaging mode, the one or more imaging demands of various scenarios may be more efficiently met, the overall imaging efficiency may be further improved, the transmission resources may be more reasonably allocated, and the user experience may be improved.

In some embodiments, the imaging condition data may be pre-calculated and stored in the imaging condition data determination module 2220, the storage device 130, or the ultrasonic device 110 by a preset imaging condition calculation program according to characteristic data of the ROI region selected in the history. In some embodiments, the preset imaging condition calculation program may be triggered to perform real-time calculation according to a currently selected ROI region.

FIG. 11 is a flowchart illustrating an exemplary process of determining imaging condition data according to a region of interest according to some embodiments of the present disclosure. In some embodiments, process 1100 may be performed by the processing device 2200, the ultrasonic device 110, and/or the processing device 120. In some embodiments, the process 1100 may be stored in a storage device (e.g., the storage device 130) in the form of programs or instructions, the process 1100 may be implemented when the ultrasonic imaging system 100 (e.g., the processing device 120) executes the program or instructions. In some embodiments, the process 1100 may be performed by one or more of the modules in FIG. 7, FIG. 22, and/or FIG. 34. In some embodiments, operation 820 in FIG. 8 may be performed according to process 1100.

As shown in FIG. 11, the process 1100 may include one or more of the following operations.

In 821, an effective imaging region of a region of interest may be calculated according to the region of interest.

In 822, a first transmission condition parameter of a transmission covering the effective imaging region may be determined according to the effective imaging region.

In 823, relationship data between a first transmission condition parameter corresponding to the effective imaging region and a second transmission condition parameter corresponding to the global imaging region.

In some embodiments, the first transmission condition parameter and the second transmission condition parameter may include at least the count of focal points or the count of transmission times, respectively. The count of focal points or the count of transmission times is the parameter that may reflect the transmission condition during the imaging. If at least the count of focal points or the count of transmission times is selected as the first transmission condition parameter corresponding to the effective imaging region and the second transmission condition parameter corresponding to the global imaging region, then the relationship data between the first transmission condition parameter corresponding to the effective imaging region and the second transmission condition parameter corresponding to the global imaging region may be obtained to determine the imaging condition data accurately and efficiently, thereby ensuring the reliability of data acquisition. In some embodiments, the first transmission condition parameter or the second transmission condition parameter may further include a transmission time interval, etc.

As shown in FIG. 10a, in some embodiments, after selecting the ROI, the area of the effective imaging region may be calculated according to a preset ratio range (such as 1.5 to 1.8) or a preset ratio of the area of the effective imaging region to the ROI area, and the effective imaging region (e.g., in a regular shape such as a solid-line rectangle shown in FIG. 10a) may be determined through the touch screen and/or non-touch screen operation instruction according to the area of the effective imaging region.

As shown in FIG. 10b, in some embodiments, when the selected ROI is a non-closed shape, such as a non-closed line segment, through the touch screen and/or non-touch screen operation instruction, according to a preset area multiple, the non-closed shape may be thickened within a preset area multiple range (such as 1.8 to 2.0, or greater than 2.0) to determine the effective imaging region (e.g., in an irregular shape such as a solid line shape shown in FIG. 10b).

Therefore, for the effective imaging region determined according to the selected ROI, as long as an outward extended area meets the preset area condition, whether the specific shape is a closed shape or a non-closed shape, a regular shape or an irregular shape may be determined according to a specific need of the ultrasonic detection user or the doctor user.

In some embodiments, the count of focal points (which may be denoted by Sroi) required to cover the effective imaging region may be calculated according to Equation (1):

$$Sroi = \frac{Wroi}{Worg} * Sorg * ts , \qquad ( 1 )$$

wherein Worg may be a width of the global imaging region, Wroi may be a width of the effective imaging region, Sorg may be the count of transmission times required for the global imaging region, and is may be a scale factor.

FIG. 12a is a schematic diagram illustrating exemplary focal points required in transmission to cover a global imaging region according to some embodiments of the present disclosure. FIG. 12b is a schematic diagram illustrating exemplary focal points required in transmission to cover an effective imaging region according to some embodiments of the present disclosure. Taking the focused transmission mode as an example, the solid line frame region shown in FIG. 12b may be the effective imaging region of the region of interest, the focal points may be distributed in the effective imaging region, and a line scanning manner is adopted. In some embodiments, the width of the effective imaging region Wroi may be calculated according to a foregoing calculation manner of the effective imaging region. In some embodiments, the width of the global imaging region Worg and the count of transmission times required for the global imaging region Sorg may be equal to a width of the initial image and the count of transmission times required for the initial image, respectively.

FIGS. 13a-13b are schematic diagrams illustrating exemplary focal points required in transmission to cover an effective imaging region according to some embodiments of the present disclosure. Different from the way in which the focal points are distributed in the effective imaging region shown in FIG. 12a and FIG. 12b, the focal points shown in FIG. 13a and FIG. 13b may be distributed outside the effective imaging region, the transmission aperture(s) may be selected appropriately so that the acoustic energy may be mostly distributed in the effective imaging region, which may require less count of transmission times than that for the focal points being distributed in the effective imaging region (e.g., for a focal point F1 in FIG. 13a, the count of required focus points is lesser).

In some embodiments, the transmission focal point position corresponding to the effective imaging region may be determined as follows: an ellipse may be established at a center of the effective imaging region (i.e., a center of the rectangular region in FIG. 13A), and the long axis and short axis of the ellipse may be 1.5-2 times the width and height of the rectangular region respectively; the focal point(s) may be located below the effective imaging region and evenly distributed on the ellipse, and a distance between a focal point on the two sides may be greater than the width of the effective imaging region; an array directly above the effective imaging region may be selected as transmission aperture(s), at this time, the directivity of corresponding transducer array element(s) is good. In this example, the size of the transmission aperture may be slightly larger than the width of the effective imaging region. An actual transmission aperture size may be determined and set according to the actual desired effect.

In some embodiments, each transmission may cover at least part of the effective imaging region. As shown in FIG. 13b, assuming that the white shadow area AF is the effective transmission region of the focal point F, the effective imaging region may be expressed as Aroi, and an intersection of the two regions may be a region where the transmission of the focal point F may be used in imaging (referred to an actual imaging region of the focal point F later) (such as the region AF1 enclosed by black line segments in FIG. 13b). The actual imaging region of other focal points may be calculated in the same or similar way. If a union of the actual imaging regions of all focal points is Aroi, an ultrasonic transmission may cover the effective imaging region. In some embodiments, the count of focal points required to meet the above conditions may be calculated as follows: a minimum length that a middle focal point may cover in the effective imaging region (i.e., a line segment mn obtained by using two illumination boundaries of the focal point to intersect a boundary of the effective imaging region) may be calculated. Specifically, Lmn may be calculated according to Equation (2):

$$Lmn = \frac{Lt}{L s} * St , \qquad ( 2 )$$

wherein Ls may be a distance from the focal point F1 to a nearest boundary of the effective imaging region, Lt may be a distance from the focal point F1 to the transducer, St may be a size of the transmission aperture.

Further, the count of required transmission times of the effective imaging region may be calculated according to Equation (3):

$$Sroi = \frac{Wroi}{mn} * ts , \qquad ( 3 )$$

wherein Wroi may be a width of the effective imaging region, ts may be the same as in Equation (1), i.e., a scale factor.

In some embodiments, since the main parameters affecting the frame rate in the ultrasonic system may include a detection depth and/or the count of transmission times required for generating one image frame, a maximum frame rate of the system may be calculated according to Equation (4) (in which a maximum frame rate may be expressed as forg):

$$forg = \frac{c}{2 * Sorg * h} , \qquad ( 4 )$$

wherein h may be a detection depth, Sorg may be the count of transmission times required for generating one frame of an initial image covering the global imaging region, and c may be a speed at which acoustic wave(s) travel in a medium, i.e., a speed of the sound.

According to Equation (1) or Equation (3), the count of required transmission times Sorg of the effective imaging region may be calculated. If the effective imaging region is imaged, a minimum frame rate of the system may be calculated according to Equation (5) (in which a minimum frame rate is expressed as froi):

$$froi = \frac{c}{2 * ( Sorg + Sroi ) * h} . \qquad ( 5 )$$

In some embodiments, a ratio of an optimized frame rate of the effective imaging region to the frame rate of the global imaging region (which may be denoted by r), i.e., the relationship data between the first transmission condition parameter of the effective imaging region and the second emission condition parameter of the global imaging region may be calculated according to Equation (6):

$$r = \frac{froi}{forg} = \frac{Sorg}{Sorg + Sroi} . \qquad ( 6 )$$

FIG. 14 is a flowchart illustrating an exemplary process of determining the target optimized imaging mode based on the imaging condition data and triggering the imaging operation in the target optimized imaging mode according to some embodiments of the present disclosure. In some embodiments, process 1400 may be performed by the processing device 2200, the ultrasonic device 110, and/or the processing device 120. In some embodiments, the process 1400 may be stored in a storage device (e.g., the storage device 130) in the form of a program or instruction, and the process 1400 may be implemented when the ultrasonic imaging system 100 (e.g., the processing device 120) executes the program or instruction. In some embodiments, the process 1400 may be performed by one or more modules in FIG. 7, FIG. 22, and/or FIG. 34. In some embodiments, operation 830 in FIG. 8 may be performed according to the process 1400.

In some embodiments, the process 1400 may include one or more of the following operations.

In 831, whether relationship data between a first transmission condition parameter of an effective imaging region and a second transmission condition parameter of a global imaging region is less than a threshold may be determined.

In 832a, in response to the relationship data being less than the threshold, imaging operation in the first optimized imaging mode may be triggered. In some embodiments, the imaging operation in the first optimized imaging mode may include: adjusting the transmission parameter under a condition of meeting the frame rate demand, to enhance an acoustic energy of the region of interest.

In 832b, in response to the relationship data being no less than the threshold, imaging operation in the second optimized imaging mode may be triggered. In some embodiments, the imaging operation in the second optimized imaging mode may include: performing the synchronous operation of an enhanced imaging covering the region of interest and a non-enhanced imaging covering the global imaging region to obtain an enhanced image of the region of interest and a global image relating to the global imaging region; performing an image compounding operation on the enhanced image and the global image.

In some embodiments, in 832a, in response to the relationship data being less than the threshold, imaging operation in the first optimized imaging mode may include: adjusting the transmission parameter of the transmission to maintain an original acoustic energy of the global imaging region while meeting the frame rate demand to enhance the acoustic energy of the region of interest.

In some embodiments, in 832b, in response to the relationship data being no less than the threshold, imaging operation in the second optimized imaging mode may include: adjusting the transmission parameter of the transmission under the condition of meeting the frame rate demand to enhance the acoustic energy of the global imaging region including the region of interest.

According to the relationship between the threshold and the relationship data between the transmission condition parameter of the effective imaging region and the transmission condition parameter of the global imaging area, whether the effective imaging region of the currently selected ROI belongs to a large region or a small region may be determined, and a corresponding optimized imaging mode may be triggered according to the region characteristics.

For example, when the relationship data is less than the threshold, which may indicate that a current effective imaging region is large, if enhancing the effective imaging region covering the region of interest, the count of transmission times may be increased or the transmission time duration may be lengthened, thereby further affecting the frame rate. Therefore, on the basis of the first optimized imaging mode covering the global imaging region, a corresponding transmission parameter may be adjusted for the region of interest to increase the acoustic energy of the region of interest or the acoustic energy of the global imaging region, which may not only improve the imaging quality without reducing the frame rate, but also realize the global imaging and dynamic adjustment of the global imaging region, and improve the imaging efficiency. When the relationship data is greater than the threshold, which may indicate that a current effective imaging region is small, the enhanced imaging covering the region of interest may not cause too many transmission times or too long transmission time duration. In the enhanced imaging operation covering the region of interest, the non-enhanced imaging operation covering the global imaging region may be performed at the same time, and the count of transmission times or the transmission time duration may be controlled within a reasonable range. Finally, the frame rate may be guaranteed while improving the imaging quality, so that the final imaging efficiency may be greatly improved.

In some embodiments, in operation 831, it may be determined whether the relationship data r calculated by Equation (6) is less than the threshold T. In some embodiments, the threshold T may be set according to specific imaging demand(s). In some embodiments, the one or more imaging demands may include frame rate demands used as a key parameter for determining the threshold T, such as a minimum frame rate demand, etc. In different imaging scenes, the frame rate demands may be different. For example, a carotid artery examination has low demands for the frame rate, and the minimum frame rate may be no less than 20 frames per second (fps), while a cardiac examination has high demands for the frame rate, and the minimum frame rate may be no less than 50 fps.

Exemplarily, assuming the detection depth may be 10 cm, the focused transmission is used, and ultrasonic waves may need to be transmitted 128 times for each frame in the global imaging region imaging, then the maximum frame rate that the ultrasonic system achieves may be 60 fps. After optimizing local imaging, if the imaging frame rate is required to be no less than 40 fps, then the threshold may be

$$T = \frac{40}{60} \approx 0.67 ,$$

and theoretically, the maximum count of transmission times of the effective imaging region that can be achieved may be 63. According to the above Equation (6):

$$\frac{Sorg}{Sorg + Sroi} = 0.67 , Sorg = 128 , Sroi = 63$$

may be calculated, which means that the count of transmission times for the effective imaging region is no more than 63.

In some embodiments, a balance between imaging quality and frame rate may be achieved by adjusting the scale factor ts, i.e., ts is variable and may be set and adjusted in real time according to different needs. In some embodiments, when the user pays more attention to the imaging quality, the scale factor ts may be appropriately increased. In some embodiments, when the user has high demands for the frame rate, the scale factor ts may be appropriately reduced.

Exemplarily, if the initial ts is set to 1, the transmission with the count of transmission times may just cover an entire effective imaging region. If the imaging quality is required to be higher, ts may be appropriately increased to 1.2 to 1.5, but the frame rate may be reduced. If there is a higher demand for the frame rate, the parameter may be appropriately adjusted to about 0.8. For example, assuming that the global imaging region is imaged using a linear array, the width of the global imaging region is 5 cm, and a calculated width of the effective imaging region is 1.5 cm, then the minimum count of transmission times required to cover the effective imaging region may be obtained according to Equation (1) as:

$$Sroi = \frac{Wroi}{Worg} * Sorg = \frac{1.5}{5} ⨯ 128 \approx 38 .$$

Assuming that the imaging quality may be improved by increasing the count of transmission times under a same condition for the threshold t calculation above, ts may be increased to 1.2 to 1.6 without affecting the frame rate. If continue to increase ts, the system may provide a reminder that the current state may reduce the minimum frame rate, and the doctor may need to choose whether to continue to increase the scale factor.

In some embodiments, when performing operation 832a, in response to the relationship data being less than the threshold, the imaging operation in the first optimized imaging mode may be triggered, the transmission parameter may be adjusted under the condition that the frame rate demands are met, in which one type of transmission parameter may be adjusted individually or several types of transmission parameters may be adjusted synchronously.

In some embodiments, the transmission parameter may include a transmission mode, a transmission aperture parameter, a transmission focal point parameter, a transmission declination angle, a transmission frequency, a transmission waveform, or a gain (i.e., a gain adjustment), or the like, or a combination thereof. In some embodiments, the transmission mode may include at least one or a hybrid of two or more transmission modes of focused wave transmission with different transmission frequencies, diverging wave transmission, wide beam transmission, plane wave transmission, and single-array element transmission, or any other available transmission mode. In some embodiments, the transmission aperture parameter may include a transmission aperture position or a combination mode of transmission array elements, a distance between transmission apertures, a distance between receiving apertures, or any other feasible transmission aperture parameter. In some embodiments, the transmission focal point parameter may include the count of transmission focal points, transmission focal point position(s), transmission focal point depth(s), or any other feasible transmission focal point parameter. In some embodiments, the transmission waveform may be a sine waveform, a shock waveform, a waveform of any shape obtained from a superposition of waves with multiple frequencies, or any other feasible transmission waveform.

Exemplarily, in a certain scene with high demands on the frame rate (e.g., the cardiac examination), the frame rate may not be sacrificed greatly, which may mean that when performing the imaging operation on the effective imaging region, the increment of the count of transmission times may be limited. At this time, the focal point(s) may be set outside the effective imaging region for imaging. If the effective imaging region is wide, the effective transmission region may be expanded by expanding the transmission aperture and increasing a transmission declination angle. If the effective imaging region is in the near-field region (i.e., the region close to the probe), the transmission frequency of the effective imaging region may be increased, so that high-frequency acoustic wave(s) can produce the desired imaging effect on the near-field region.

FIGS. 15a-15b are schematic diagrams illustrating adjusting a transmission parameter according to some embodiments of the present disclosure. As shown in FIG. 15a, a focused wave transmission manner without declination angle may be adopted, and the focal points may be distributed horizontally within a width W range of the effective imaging region. In order to ensure the imaging effect of the edge, the focal point distribution range may be appropriately expanded, such as the range of 1.2 to 1.5 times the width W, a longitudinal position of the focal points is at the position of L multiplies the depth of the effective imaging region, i.e.,

$$L = \frac{FT}{OA} ,$$

wherein the value of L may be from 2 to 3, and the count of focal points may be determined according to one or more of the aforementioned Equations (1), (2), (3), (4), (5) and (6). As shown in FIG. 15b, the focal point position shown in FIG. 15a, which is located below the effective imaging region (e.g., the rectangular region shown in FIG. 15a), may be adjusted to be above the effective imaging region to make the focal point position be closer to the transmission elements of the transducer, so as to adjust the energy distribution of the acoustic wave(s) in various regions covering the global transmission region.

As shown in FIG. 13a and FIG. 13b, multi-dimensional focal point adjustment may also be achieved through a control program, and the focal point position may be adjusted in multiple dimensional spaces (e.g., a two-dimensional space, etc.) (e.g., the focal point position may be adjusted in a horizontal or vertical dimension). For example, the focal point position distribution shown in FIG. 13a and FIG. 13b may be adjusted to the focal point position distribution shown in FIG. 15a or FIG. 15b to meet the focal point adjustment demands of various imaging scenarios and ultimately improve the imaging efficiency.

By adjusting one parameter or multiple parameters in the transmission mode, a transmission aperture parameter, a transmission focal point parameter, a transmission deflection angle, a transmission frequency, a transmission waveform, and/or gain, etc., the preset imaging demands covering the global imaging region may be achieved better, which may not only achieve dynamic display and adjustment of the global imaging region, but also improve the overall imaging efficiency to meet the diverse imaging needs of the user.

In some embodiments, the adjusting transmission parameter may include: analyzing the focal point distribution in a region outside the region of interest to determine a compensation region which meets a preset focal point distribution condition; performing one or more additional transmissions to perform an ultrasonic wave energy compensation on the compensation region.

In the traditional ultrasonic imaging, the focal point distribution and imaging effect of the whole global imaging region are rarely concerned from a global perspective, especially for the regions outside the region of interest, the focal point distribution in the regions outside the region of interest may be sparse. Therefore, in some embodiments of the present disclosure, a focal point sparsity analysis may be performed on the focal point distribution in the region outside the region of interest to determine a compensation region which meets the preset focal point distribution condition. The one or more additional transmissions may be performed by adjusting the corresponding transmission parameter(s) to perform an ultrasonic wave energy compensation on the compensation region, so that the optimized image covering the global imaging region may be generated in the first optimized imaging mode, which may enhance the acoustic energy in the region of interest as much as possible without losing the reasonable demand of acoustic energy in other regions, realize a relative balance between the imaging quality, the frame rate, and other parameters, improve the imaging efficiency, and achieve the corresponding imaging effect to meet the expected imaging demands.

In some embodiments, when performing acoustic energy compensation in the compensation region, the corresponding transmission parameter of the transmission may be adjusted according to different acoustic energy compensation demands, including adjusting a single transmission or multiple transmission parameters in combination, for example, adjusting the transmission mode, or adjusting the transmission mode and the transmission deflection angle at the same time, etc.

FIGS. 16a-16c are schematic diagrams illustrating adjusting a transmission parameter according to some embodiments of the present disclosure. For example, when adjusting the transmission parameter for acoustic energy compensation, based on an initial transmission mode covering the global imaging region, in addition to using the focused transmission mode, a divergent wave transmission mode as shown in FIG. 16a, a wide beam transmission mode as shown in FIG. 16b, a plane wave transmission mode as shown in FIG. 16c, or any other feasible transmission mode may also be used.

It should be noted that the initial transmission mode covering the global imaging region is not limited to focused transmission. In some embodiments, according to different imaging demands, the initial transmission mode may be a focused transmission, a divergent wave transmission, a wide beam transmission, a plane wave transmission, or a hybrid transmission of at least one or two or more transmission modes of single array element transmission, or any other feasible transmission mode. In some embodiments, according to different imaging demands, the transmission mode used for acoustic energy compensation may include a focused transmission, a divergent wave transmission, a wide beam transmission, a plane wave transmission, a hybrid transmission of at least one or two or more transmission modes of single array element transmission, or any other feasible transmission mode.

Various transmission modes have their own transmission advantages, for example, the focused transmission may have characteristics of strong focused acoustic energy, and the wide beam, the divergent wave, and the plane wave may have characteristics of wide transmission coverage. During the acoustic compensation, one transmission mode may be compensated or multiple transmission modes may be selected according to the imaging needs of specific scenes to improve the imaging efficiency, achieve the expected imaging effect, and meet the personalized imaging needs of the user.

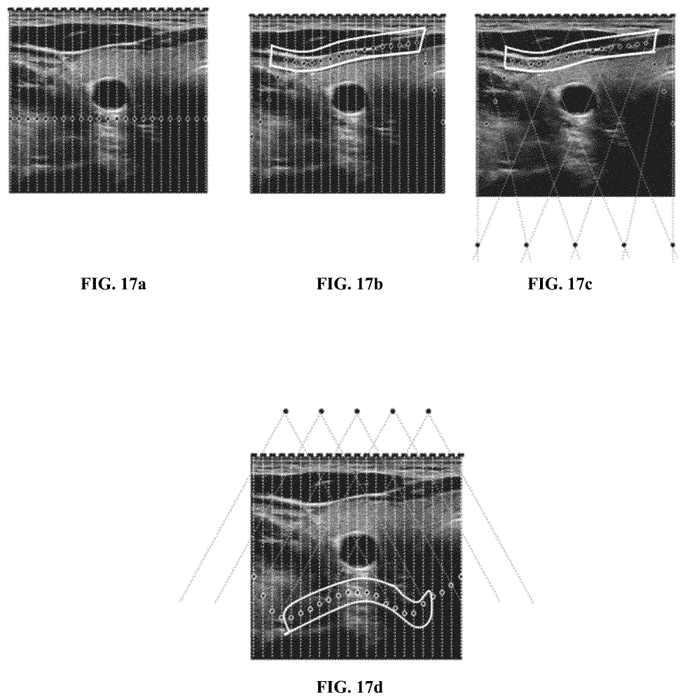

FIGS. 17a-17d are schematic diagrams illustrating adjusting a transmission parameter according to some embodiments of the present disclosure. As shown in FIG. 17a and FIG. 17b, if the selected ROI area is too large or the range across the region is wide and the second optimized imaging mode of the synchronous operation of the enhanced imaging covering the region of interest and the non-enhanced imaging covering the global imaging region is adopted, a larger count of transmission times may be needed, which may affect the frame rate. The first optimized imaging mode may be adopted to adjust the transmission parameter(s) under the condition of meeting the frame rate demands.

FIG. 17a shows the focal point distribution of the initial image, in which the focal point is evenly distributed on the same horizontal line. Suppose that the doctor selects the solid line long strip region located at the upper part of the global imaging region in FIG. 17b as the ROI, which may almost span the whole horizontal range, according to the ROI characteristics, the focal point within the original span may be moved to the ROI, and the focal point outside the ROI may be evenly distributed between the positions before and after the movement.

As shown in FIG. 17c, for a region with sparse focal point distribution in the lower part outside the ROI, the transmission may be increased appropriately, and the acoustic energy compensation operation may be performed by adding a wide beam transmission. On the basis of the initial transmission mode after adjusting the focal point position, a plurality of focal points may be added outside the global imaging region, so that the energy compensation through an additional transmission can be concentrated in the lower region of the global imaging region to compensate the energy loss in the lower region caused by the moving up focal points, so as to achieve an expected optimized imaging effect of the global imaging region.

As shown in FIG. 17d, when the selected ROI is located in the lower part of the global imaging region, it may mean that most focal points are concentrated in the far field during the transmission, and the energy of the near field may be compensated by adding a divergent wave transmission to achieve an expected optimized imaging effect of the global imaging region.

In some embodiments, in operation 832b, in response to the relationship data being no less than the threshold, triggering the imaging operation in the second optimized imaging mode and the synchronous operation of the enhanced imaging covering the region of interest and the non-enhanced imaging covering the global imaging region may include: performing the enhanced imaging on the region of interest and the non-enhanced imaging on the global imaging region using an alternate transmission to obtain the enhanced image of the region of interest and the global image of the global imaging region, respectively. In some embodiments, the alternate transmission manner may be performed, based on the same array transmission array elements at preset time points, using the alternate transmission to obtain the enhanced image of the region of interest and the global image of the global imaging region.

In some embodiments, the enhanced imaging operation of the ROI may adopt a conventional imaging manner, for example, a process similar to the imaging process of the initial image, for details, more description may be found in the related descriptions of the initial image, which may not be repeated herein. In some embodiments, the enhanced imaging operation of the ROI may adopt a focused transmission mode to achieve enhanced acoustic energy within the ROI. In some embodiments, the enhanced imaging operation of the ROI may adopt the similar imaging operation for adjusting the transmission parameter of the transmission covering the global imaging region or region of interest in the first optimized imaging mode in operation 832a mentioned above. More descriptions may be found in the related descriptions of the imaging operation of adjusting the transmission parameter of the transmission for the global imaging region or region of interest in the first optimized imaging mode, which may not be repeated herein. In some embodiments, the non-enhanced imaging operation of the global imaging region may adopt a conventional imaging manner, for example, similar to the imaging process of the initial image. More descriptions may be found in related descriptions of the initial image, which may not be repeated herein. In some embodiments, the non-enhanced imaging operation of the global imaging region may adopt the similar imaging operation of adjusting the transmission parameter of the transmission for the global imaging region or the region of interest in the first optimized imaging mode in operation 832a mentioned above. More descriptions may be found in related descriptions of the imaging operation of adjusting the transmission parameter of the transmission covering the global imaging region or the region of interest in the first optimized imaging mode, which may not be repeated herein.

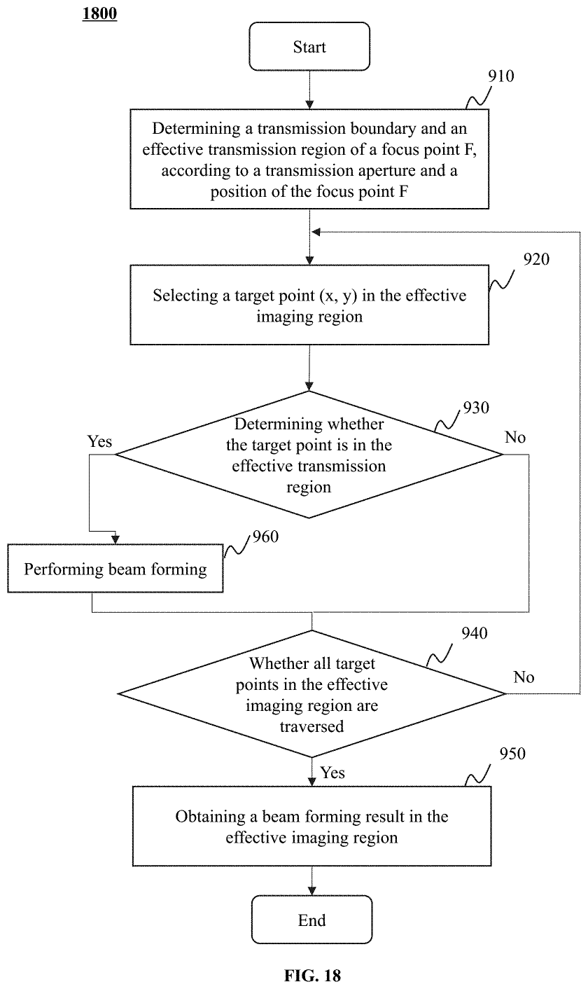

FIG. 18 is a schematic diagram illustrating an exemplary enhanced imaging operation 1800 covering a region of interest according to some embodiments of the present disclosure. In some embodiments, the process 1800 may be performed by the processing device 2200, the ultrasonic device 110, and/or the processing device 120. In some embodiments, the process 1800 may be stored in a storage device (such as the storage device 130) in the form of a program or instruction, and the process 1800 may be implemented when the ultrasonic imaging system 100 (such as the processing device 120) executes the program or instruction. In some embodiments, the process 1800 may be performed by one or more modules in FIG. 7, FIG. 22, and/or FIG. 34.

In some embodiments, the process 1800 may include one or more of the following operations.

In 910, a transmission boundary and effective transmission region of the focal point F may be calculated according to a transmission aperture and a position of focal point F.

In 920, a target point coordinate (x, y) may be selected in an effective imaging region.

In 930, whether the target point is within the effective transmission region of the focal point F may be determined.

In 940, if the target point is within the effective transmission region of the focal point F, the process may proceed to operation 960 for beam synthesis. If the target point is without the effective transmission region of the focal point F, operations 920 to 940 may be repeated until all target points are traversed.

In 950, a result of beamforming in the effective imaging region of the focal point F may be obtained.

The same or similar operations may be performed for all focal points, and the result of the beamforming for all focal points F may be compounded to obtain the result of the beamforming covering the region of interest to obtain the enhanced image of the region of interest.

In some embodiments, after obtaining the enhanced image of the region of interest and the global image of the global imaging region, the enhanced image of the ROI may be directly replaced by the ROI on the global image, an edge transition processing may be performed on the overlapping parts of the edges, such as weighted composite transition processing, to ensure that there are no obvious boundaries between the two parts, and finally obtain the optimized image under the second optimized imaging mode.

In some embodiments, exemplarily, the image compounding operation of the enhanced image and the global image may be implemented as follows: one or more operations of weighted compounding, frequency domain compounding, and edge enhancement may be performed on the enhanced image and the global image.

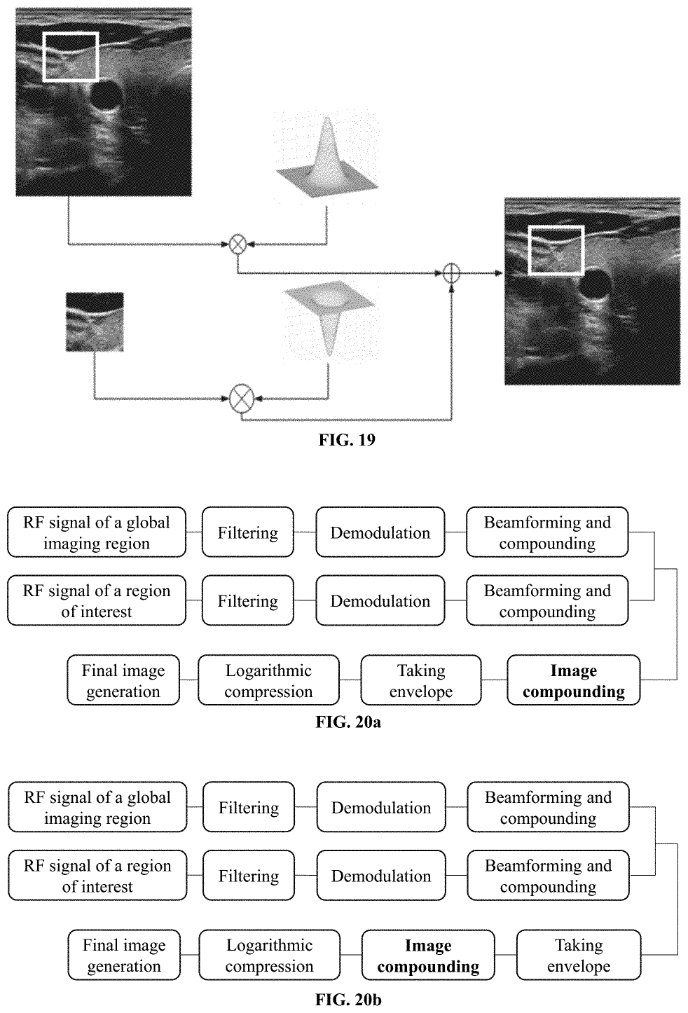

FIG. 19 is a schematic diagram illustrating an exemplary image compounding operation of an enhanced image and a global image according to some embodiments of the present disclosure. As shown in FIG. 19, in some embodiments, the image compounding operation may include: the weighted compounding operation may be performed on the enhanced image and the global image. Exemplarily, a weight parameter may be set as a Gaussian weight with the ROI as an initial point, and finally, the optimized image under the second optimized imaging mode may be obtained by the compounding operation. By optimizing the imaging quality of the image details of the image, a significant improvement may be obtained compared to the initial image.

In some embodiments, the image compounding operation may include a frequency domain compounding operation performed on the enhanced image and the global image. Exemplarily, high frequency information of the enhanced image and low frequency information may be compounded. A two-dimensional Fourier transform (or other transforms such as a wavelet transform) may be performed on the global image and enhanced image including the effective imaging region to obtain frequency domain information of the two images, a low-pass filtering may be performed on the image with high low-frequency energy, a high-pass filtering may be performed on the image with strong high-frequency signal, the inverse Fourier transform may be performed to obtain a filtered image, and then the two images may be linearly or nonlinearly compounded. The compounded image may retain strong low-frequency signals and high-frequency signals at the same time, and may include more abundant color and texture information, and thus, optimized images with better imaging effects may be obtained.

In some embodiments, the image compounding operation may include an edge enhancing operation on the enhanced image and the global image. Exemplarily, texture information may be extracted from the enhanced image (or an effective imaging region of the global image) using an edge extraction operator (a Roberts operator, a Sobel operator, a Prewitt operator, a Kirsch operator, a Robinson operator, etc.), or other edge extraction algorithms, then the texture information may be compounded to the effective imaging region (or the enhanced image) of the global image to obtain more detail information and improve the image quality, which may also ensure the good imaging effect of the optimized image.

The enhanced imaging may be performed on the region of interest by alternating transmission, and the non-enhanced imaging may be performed on the global imaging region to obtain an enhanced image on the region of interest and a global image on the global imaging region, respectively, and the enhanced image and the global image may be combined using one or more operations of weighted combination, frequency-domain combination, and edge enhancement to realize the synchronous operation of the enhanced imaging covering the region of interest and the non-enhanced imaging covering the global imaging region, which may not only improve imaging efficiency, but also guarantee the optimization of dynamic display and dynamic adjustment of the image in the global image region, thereby facilitating the viewing of the relationship between the ROI and other regions during ultrasonic testing or ultrasonic diagnosis, and meet the personalized and diversified image viewing and adjustment needs of the user.

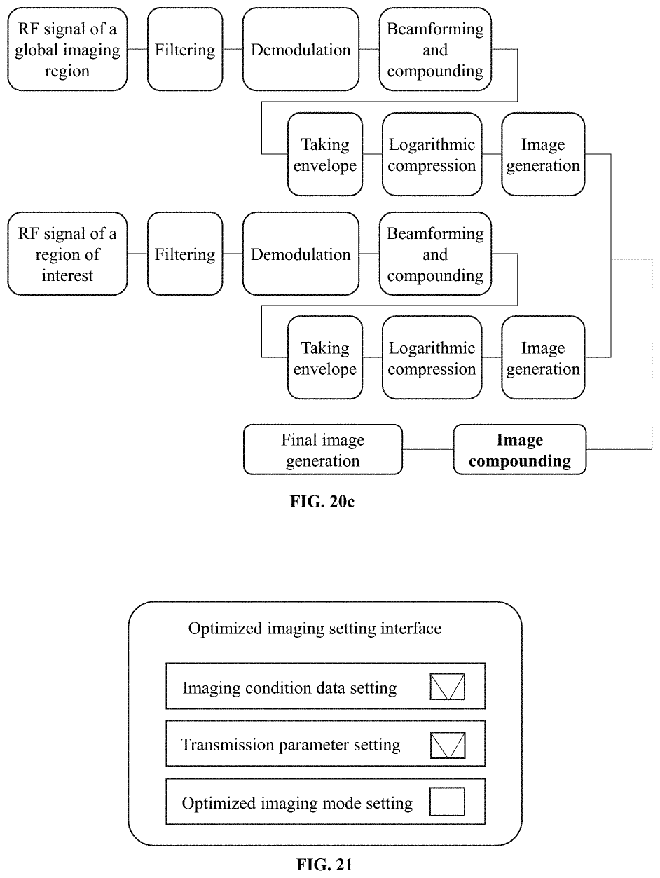

FIGS. 20a-20c are schematic diagrams illustrating an exemplary operating node of an image compounding operation of an enhanced image and a global image according to some embodiments of the present disclosure. In some embodiments, the operation node of the image compounding operation of the enhanced image and the global image may be set after the enhanced image beamforming and compounding and before the envelope taking operation as shown in FIG. 20A. In some embodiments, the compounding operation in beamforming and compounding may include hybrid imaging processing, such as line compounding, frequency compounding, space compounding, etc. In some embodiments, the operation node of the image compounding operation of the enhanced image and the global image may be set after the envelope taking operation and before logarithmic compression shown in FIG. 20b. In some embodiments, as shown in FIG. 20c, the operation node where the image compounding operation of the enhanced image and the global image is located may be set after the two images are imaged respectively and before the final imaging. Since the enhanced image and the global image have been processed into images with complete details at this operation node, the image compounding operation of the two images may obtain a better image composite effect, and finally, improve the imaging efficiency and optimize the effect.

In some embodiments, the above ultrasonic imaging process may also include: according to the optimized image, the frame rate demand, and/or a received instruction, the imaging condition data and/or the transmission parameter may be adjusted so that the user may optimize the actual effect of image, dynamic frame rate demands, or other operation instructions received to further improve the optimized image, meet the higher imaging needs of the user and improve the experience of the user.

FIG. 21 is a schematic diagram illustrating an optimized imaging setting interface according to some embodiments of the present disclosure. As shown in FIG. 21, exemplarily, after completing the execution of operations 810 to 830 and viewing the obtained optimized image, the user may wish to make further improvements and adjustments to more concerned local image regions (for example, a local region within the previously selected ROI or a local region outside the previously selected ROI), at this time, the imaging condition data may be reset by selecting “transmission condition parameter setting” (not shown in FIG. 21) in a next menu of the imaging condition data setting menu option, or reselect the ROI, and repeat the above operations 810 to 830.

Exemplarily, when the frame rate demand changes in real time during the ultrasonic imaging, the user may also adjust in real time by optimizing the imaging condition data setting menu option and/or the transmission parameter setting menu option of the imaging setting interface to make the final optimized image that meets the frame rate demands of the user.

Exemplarily, in the process of detection or diagnosis, when the ultrasonic probe moves or the user reselects the ROI, the user may receive corresponding operation instructions that a probe detection signal may change or the ROI may be drawn, the operation instructions may also trigger the pop-up of an optimized imaging setting interface, so that the user may adjust and set the corresponding imaging condition data or the transmission parameter according to the new imaging demands.

Exemplarily, during the imaging process, the user may perform an optimal imaging mode switching function operation through an optimal imaging mode switching menu option on the optimal imaging setting interface through additional instruction input according to the needs of the current optimal imaging mode. For example, switch from the first optimized imaging mode to the second optimized imaging mode.

It should be noted that the above description about the process 800 is merely provided for the purposes of illustration, and not intended to limit the scope of the present disclosure. For those skilled in the art, various modifications and changes may be made to the process 800 under the guidance of the present disclosure. However, these corrections and changes are still within the scope of the present disclosure.

FIG. 22 is a block diagram illustrating an exemplary processing device 2200 according to some embodiments of the present disclosure. As shown in FIG. 22, the processing device 2200 may include a region of interest determination module 2210, an imaging condition data determination module 2220, and/or an optimized image generation module 2230.

In some embodiments, the region of interest determination module 2210 may be used to determine a region of interest in the initial image, wherein the initial image may include a global imaging region. More descriptions about the region of interest determination module 2210 performing the determination of the region of interest on the initial image may be found in any one embodiment above of the ultrasonic imaging process 800 and the related descriptions, which may not be repeated here.

In some embodiments, the imaging condition data determination module 2220 may be used to determine the imaging condition data according to the region of interest. More descriptions may be found in any one embodiment above of the ultrasonic imaging process 800 and the related descriptions, which may not be repeated here.

In some embodiments, the optimized image generation module 2230 may be configured to determine the target optimized imaging mode based on the imaging condition data, and trigger the imaging operation in the target optimized imaging mode to generate an optimized image. The optimized imaging mode may at least include the first optimized imaging mode and the second optimize imaging mode. More descriptions about the specific process for the optimized image generation module 2230 to perform the judgment and generate the optimized image and the related descriptions may be found in any one embodiment above of the ultrasonic imaging process 800 and the related descriptions, which may not be repeated here.

An ultrasonic imaging system provided by some embodiments of the present disclosure may include: at least one storage medium storing a set of instructions; at least one processor in communication with the at least one storage medium, when executing the stored set of instructions, the at least one processor causes the system to: determine a region of interest in the initial image, the initial image may include a global imaging region; determine imaging condition data according to the region of interest; determine the target optimized imaging mode based on the imaging condition data, and trigger the imaging operation in the target optimized imaging mode to generate an optimized image, the optimized imaging mode includes a first optimized imaging mode and the second optimized imaging mode, wherein the first optimized imaging mode is configured to perform an operation of adjusting a transmission parameter covering the global imaging region, the second optimized imaging mode is configured to perform the synchronous operation of an enhanced imaging covering the region of interest and a non-enhanced imaging covering the global imaging region to obtain an enhanced image relating to the region of interest and a global image relating to the global imaging region, respectively. More descriptions about the ultrasonic imaging system may be found in FIGS. 8-21 and the related descriptions, which may not be repeated here.

Further, an ultrasonic imaging device provided by some embodiments of the present disclosure may include a processor, wherein the processor is configured to execute the corresponding process of the ultrasonic imaging process 800 according to any of the foregoing embodiments, more descriptions may be found in FIGS. 8-21 and the related descriptions, which may not be repeated here.

Further, a non-transitory computer readable medium provided by some embodiments of the present disclosure including executable instructions, the instructions, when executed by at least one processor, causing the at least one processor to effectuate a corresponding process of the ultrasonic imaging process 800 according to any of the foregoing embodiments, more descriptions may be found in FIGS. 8-21 and the related descriptions, which may not be repeated here.

The ultrasonic imaging method, system, device, and computer-readable storage medium provided by some embodiments of the present disclosure have at least the following beneficial effects: (1) comprehensively consider the relationship factors that affect the overall imaging efficiency such as the imaging quality, the frame rate, the transmission time, etc., for the imaging demands or diagnostic demands of different imaging conditions, analyze and determine according to the corresponding imaging condition data of the selected ROI, the target optimized imaging mode may be determined based on the imaging condition data and the imaging operation in the target optimized imaging mode may be triggered to provide a corresponding imaging solution with high adaptability, which may not only improve the imaging efficiency but also meet a variety of different imaging needs, so as to provide a best imaging solution while overcoming the shortcomings of the previous technical solutions; (2) supports the imaging mode of the global region in which the adaptively enhanced region of interest (hereinafter referred to as ROI) and other regions may be dynamically displayed and adjusted at the same time, which is convenient for the user to view and adjust the image during detection or diagnosis, which may improve the user experience; (3) due to calculations involved in the ultrasonic imaging process such as the region of interest selection or imaging condition data calculations, it may be calculated and stored in a preset way, or it may be calculated only after the user needs to provide an interactive way (such as an interactive interface, etc.) for the user to dynamically adjust, which may reduce the amount of calculation, meet the real-time interaction and dynamic adjustment needs of users during imaging, and further improve the user experience.

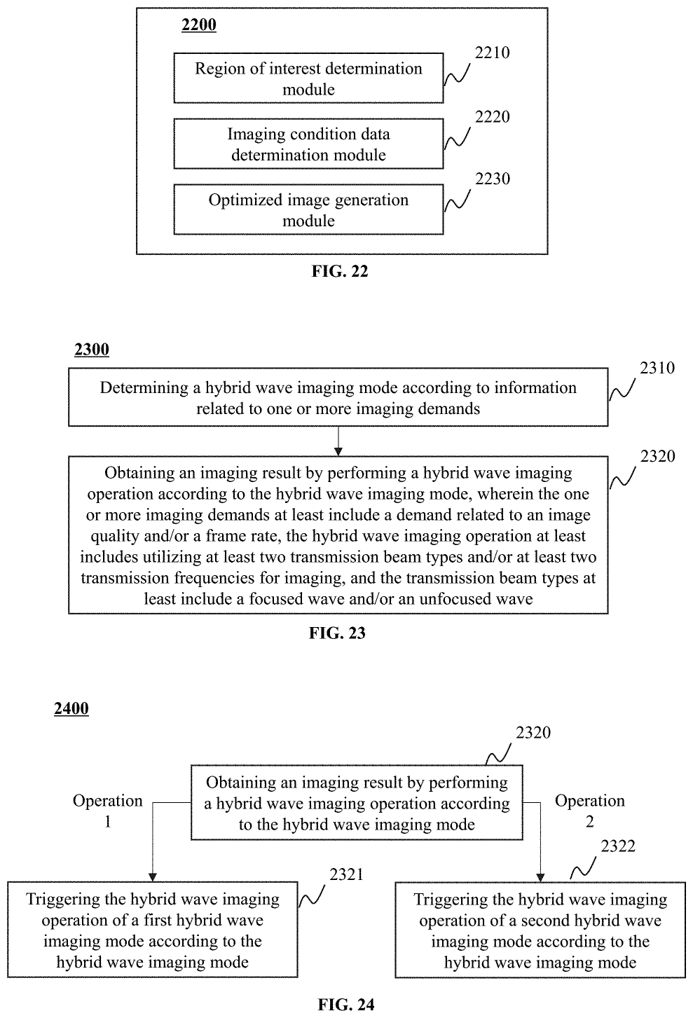

FIG. 23 is a flowchart illustrating an exemplary ultrasonic imaging process 2300 according to some embodiments of the present disclosure. In some embodiments, the process 2300 may be performed by the ultrasonic device 110, the processing device 120, and/or the processing device 3400. In some embodiments, the process 2300 may be stored in a storage device (such as the storage device 130) in the form of a program or instruction, and the process 2300 may be implemented when the ultrasonic imaging system 100 (such as the processing device 120) executes the program or instruction. In some embodiments, the process 2300 may include one or more of the following operations.

In 2310, the corresponding hybrid wave imaging mode may be determined according to the information related the one or more imaging demands. In some embodiments, operation 2310 may be performed by the hybrid wave imaging mode determination module 3410.

In 2320, the corresponding hybrid wave imaging operation may be performed to obtain the corresponding imaging result according to the hybrid wave imaging mode. In some embodiments, the operation 2320 may be performed by the hybrid wave imaging operation module 3420.

In some embodiments, the one or more imaging demands may include the one or more demands related to the image quality and/or the frame rate. In some embodiments, the information related to the one or more imaging demands may include a spatial resolution, a contrast resolution, a temporal resolution, an image signal-to-noise ratio, a frame rate, an imaging speed, an imaging time, or any other feasible index (or an imaging demand parameter) that may reflect the demands related to the image quality and/or the frame rate. In some embodiments, the information related to the one or more imaging demands may be obtained by setting or adjusting the corresponding imaging demand parameter in the ultrasonic system. In some embodiments, the information related to the one or more imaging demands may be obtained by manual input, such as receiving an imaging demand instruction from the user in real time.

In some embodiments, the transmission beam types may include the focused wave and/or the unfocused wave, and/or any other feasible beam type. In some embodiments, the unfocused wave may include a plane wave, a divergent wave, a wide beam, or any other feasible unfocused beam type. In some embodiments, the hybrid wave imaging mode may include imaging modes with the same transmission frequency and different transmission beam types, such as a hybrid wave imaging mode in which both the divergent wave and the focused wave are at the same transmission frequency (e.g., 8 MHz). In some embodiments, the hybrid wave imaging mode may include imaging modes with different transmission frequencies and the same transmission beam type, such as the hybrid wave imaging mode of two focused waves or two unfocused waves with transmission frequencies of 8 MHz and 4 MHz, respectively. In some embodiments, the hybrid wave imaging mode may include imaging modes with different transmission frequencies and different transmission beam types, for example, a hybrid wave imaging mode of a divergent wave with a transmission frequency of 8 MHz, a focused wave with a transmission frequency of 10 MHz, and a plane wave with a transmission frequency of 5 MHz. It should be noted that in the hybrid wave imaging mode, the counts of transmission times of the corresponding transmission beams of the different transmission beam types and/or different transmission frequencies may be not specially limited. For example, in a specific hybrid wave transmission mode, plane waves with the same transmission frequency may be transmitted and imaged for multiple times at multiple different time nodes or cycles, or only once at one time node or cycle. As another example, divergent waves with different transmission frequencies may be transmitted twice or more at different time nodes or cycles. As a further example, the divergent wave, the focused wave, and the plane wave with different transmission frequencies may be imaged once respectively. In addition, it should be noted that in the hybrid wave imaging mode, the sequence of corresponding beam transmission for the different transmission beam types and/or different transmission frequencies may be not particularly limited. For example, transmitting the divergent wave first and then the focused wave. As another example, transmitting a divergent wave with a frequency of 8 MHz first, then transmitting a focused wave with a frequency of 10 MHz, and then transmitting a plane wave with a frequency of 5 MHz, etc.

In some embodiments, the hybrid wave imaging mode may include the first hybrid wave imaging mode and/or the second hybrid wave imaging mode. In some embodiments, the first hybrid wave imaging mode may be used for the full aperture hybrid transmission operation, and the second hybrid wave imaging mode may be used for the moving aperture hybrid transmission operation.

In some embodiments, the operation of the full aperture hybrid transmission may be a transmission operation in which all apertures of the array elements participate in the transmission during the focused wave and/or the unfocused wave transmission to cover a large scanning region while improving the frame rate due to a fast imaging speed, a wide coverage, a uniform acoustic field, and less count of transmission times of the unfocused wave, the image quality may be improved through the enhancement of the focused wave energy to more effectively meet the expected imaging needs of the user. In some embodiments, the full aperture hybrid transmission operation may be the unfocused wave transmission in which all apertures participate in the transmission, and the local aperture (i.e., the partial aperture) may participate in the transmission when the focused wave is transmitted (e.g., the transmission is focused on a specific region or the region of interest with high imaging quality demands) to allocate resources reasonably, and save costs while meeting the imaging needs of the user.

In some embodiments, the operation of the moving aperture hybrid transmission may be a transmission operation in which all apertures or local apertures of the array elements are used in transmission according to a corresponding hybrid transmission order when the focused wave and/or the unfocused wave are transmitted. In some embodiments, the operation of the moving aperture hybrid transmission may be a transmission operation in which both the focused wave (e.g., the focused wave) and the unfocused wave (e.g., the diverging waves) are transmitted using local apertures according to a corresponding hybrid transmission order. In some embodiments, during the operation of the moving aperture hybrid transmission, the focused wave(s) and the unfocused wave(s) may be alternately transmitted according to a corresponding hybrid transmission order. In some embodiments, the order, the rule, or the procedure for hybrid transmissions may include individual transmission time node settings and/or alternate transmission time interval settings for the focused wave(s), the unfocused wave(s), or the like. The operation of the moving aperture hybrid transmission may be used to transmit according to a specific combination order through different hybrid beams under a correspondingly set transmission order or rule, which may make comprehensive use of the respective advantages of multiple beams to transmit and scan in important regions (such as the region of interest) to obtain hybrid wave echo data with richer information (such as echo signal data, echo image, or imaging data of multiple beams), which may be convenient for subsequent echo signal recombination or image recombination processing, so as to provide guarantees for meeting the personalized imaging needs of different users and different scenes.

FIG. 24 is a flowchart illustrating an exemplary hybrid wave imaging operation in a hybrid wave imaging mode according to some embodiments of the present disclosure. In some embodiments, process 2400 may be performed by the processing device 3400, the ultrasonic device 110, and/or the processing device 120. In some embodiments, the process 2400 may be stored in a storage device (such as the storage device 130) in the form of a program or instruction, and the process 2400 may be implemented when the ultrasonic imaging system 100 (such as the processing device 120) executes the program or instruction. In some embodiments, the process 2400 may be performed by one or more modules in FIGS. 7, 22, and/or 34. In some embodiments, operation 2320 in FIG. 23 may be performed according to the process 2400.

As shown in FIG. 24, the operation 2320 may perform the corresponding hybrid wave imaging operation according to the hybrid wave imaging mode to obtain the corresponding imaging result, which may include at least one of the following two branch operations:

In 2321, triggering the hybrid wave imaging operation in the first wave imaging mode according to the hybrid wave imaging mode may include: determining the position of the first focal point, wherein the position of the first focal point may satisfy the boundary condition of the first focal point; determining a first transmission parameter of the first hybrid wave imaging mode according to the position of the first focal point; performing the full aperture transmission operation of a first ultrasonic wave and the full aperture transmission operation or operation of local aperture transmission of a second ultrasonic wave to obtain first hybrid wave echo data according to the first transmission parameter of the first hybrid wave imaging mode.

In 2322, triggering the hybrid wave imaging operation in the second hybrid wave imaging mode according to the hybrid wave imaging mode may include: determining the position of the second focal point, wherein the position of the second focal point may satisfy the boundary condition of the second focal point; determining a second transmission parameter of the second hybrid wave imaging mode according to the position of the second focal point; performing the full aperture transmission operation of a first ultrasonic wave and the full aperture transmission operation or the local aperture transmission operation of a second ultrasonic wave to obtain second hybrid wave echo data according to the second transmission parameter of the second hybrid wave imaging mode.

In some embodiments, the position of the first focal point may include an arrangement position of at least part (e.g., all) of the focal points and/or respective positions of at least part (e.g., all) of the focal points of each transmission beam in the first hybrid wave imaging mode, and the position of the second focal point may include an arrangement position of at least part (e.g., all) of the focal points and/or respective positions of at least part (e.g., all) of the focal points of each transmission beam in the second hybrid wave imaging mode. In some embodiments, the first focal point may include a real focal point and/or a virtual focal point, and the second focal point may include a real focal point and/or a virtual focal point. In some embodiments, the first focal point may be located within and/or outside the imaging region, and the second focal point may be located within and/or outside the imaging region. In some embodiments, the count of the first focal points may be one or more, and the count of the second focal points may be one or more. For example, the first focal points or the second focal points may include a plurality of (e.g., 10) real focal points (of focused waves) located within the imaging region and a plurality of (e.g., 8) virtual focal points (of divergent waves) located outside the imaging region.

In some embodiments, the first transmission parameter may include a respective delay time and/or a deflection angle of at least part (e.g., all) of the focal points in the first hybrid wave imaging mode, and the second transmission parameter may include a respective delay time and/or a deflection angle of at least part (e.g., all) of the focal points in the second hybrid wave imaging mode. In some embodiments, the first ultrasonic wave may be the unfocused wave and the second ultrasonic wave may be the focused wave. In some embodiments, the first hybrid wave echo data or the second hybrid wave echo data may include respective echo data of different beams or respective echo compounding data of different beams. In some embodiments, the respective echo data of different beams may include echo signal data or echo image data of the corresponding beams. In some embodiments, the respective echo compounding data of different beams may include the echo compounding data of the echo signal of the corresponding beam generated through the beamforming.

In some embodiments, the first hybrid wave imaging mode or the second hybrid wave imaging mode may be triggered (or performed) separately. In some embodiments, the first hybrid wave imaging mode and the second hybrid wave imaging mode may be triggered simultaneously.

By triggering the first hybrid wave imaging mode or the second hybrid wave imaging mode, under the respective hybrid wave imaging mode, according to the corresponding transmission characteristics of the full aperture transmission operation or the moving aperture transmission operation, and through a boundary condition of the corresponding focal point(s), the corresponding position(s) of the focal point(s) and the corresponding transmission parameter(s) that may be used to effectively perform the full aperture transmission operation or the moving aperture transmission operation may be determined. It may be more advantageous to carry out the full aperture transmission operation or the moving aperture transmission operation of the hybrid wave to ensure the imaging efficiency and satisfy a variety of different user imaging needs.

In some embodiments, the hybrid wave imaging operation of the hybrid imaging mode may further include: different types of the transmission beams, and/or the hybrid wave with different transmission frequencies may be used for imaging for different local imaging regions in the global imaging region. In some embodiments, different local imaging regions in the global imaging region may be imaged using the first hybrid wave imaging mode and/or the second hybrid wave imaging mode. For example, a local imaging region may adopt the first hybrid wave imaging mode, and another local imaging region may adopt the second hybrid wave imaging mode. Therefore, the hybrid wave imaging mode may be set according to the specific imaging demands of the local imaging region(s), which may further improve the effectiveness and efficiency of imaging and meet the imaging demands of complex scenes.

In some embodiments, the hybrid wave imaging operation of the hybrid wave imaging mode may further include: performing the operation of adjusting transmission parameter(s) covering the global imaging region, or, the synchronous operation of enhanced imaging covering the local imaging region and non-enhanced imaging covering the global imaging region. In some embodiments, it may be determined, according to the imaging condition data, in the first hybrid wave imaging mode or the second hybrid wave imaging mode, whether performing the transmission parameter adjustment operation covering the global imaging region according to the region of interest, or the synchronous operation of the enhanced imaging covering the region of interest and the non-enhanced imaging covering the global imaging region. In some embodiments, it may be determined, according to the relationship data between the transmission condition data of ROI and the transmission condition data of the global imaging region, in the first hybrid wave imaging mode or the second hybrid wave imaging mode, whether performing the transmission parameter adjustment operation covering the global imaging region according to the region of interest, or the synchronous operation of the enhanced imaging covering the region of interest and the non-enhanced imaging covering the global imaging region.

Exemplarily, if the count of required focal points, the count of the transmission times, or the transmission interval of the selected ROI is small, the synchronous operation of the enhanced imaging covering the ROI and the non-enhanced imaging covering the global imaging region may be performed in the second hybrid wave imaging mode. Exemplarily, if the ROI area or the ratio of the ROI area to the area of the global imaging region is large, it may indicate that the ROI accounts for a large proportion in the global imaging region, and the transmission parameter adjustment operation covering the global imaging region may be performed in the first hybrid wave imaging mode.

By further combining the corresponding imaging operations in different regions (e.g., according to the imaging condition data and other characteristics of the region of interest) under different hybrid wave imaging modes, it may meet the imaging demands of a variety of scenes more efficiently (especially complex imaging scenes with different needs in different regions), further improve the imaging efficiency as a whole, allocate the transmission resources more reasonably, and improve the user experience.

In some embodiments, the boundary condition of the first focal point and/or the boundary condition of the second focal point may be obtained by: determining a limiting deflection angle and/or a limiting delay time of the transmission beams in the first hybrid wave imaging mode and/or the second hybrid wave imaging mode according to the array element directional restriction condition; determining the boundary condition of the first focal point and/or the boundary condition of the second focal point inside and outside the imaging region according to the limiting deflection angle and/or the limiting delay time.

In some embodiments, the array element directional restriction condition may be a restriction determined according to an array element directional function. In some embodiments, the array element directional function may include a spatial distribution function (e.g., a directivity diagram or a directional characteristic function) reflecting a radiated acoustic field of the transmission array element (or transducer) or sensitivity of the receiving array element (or the transducer). In some embodiments, the array element directional restriction condition may be changed by setting or adjusting a corresponding parameter of the array element (e.g., an array element aperture, an array element center distance, an array element width, a count of array elements, or an array element transmission frequency, etc.). In some embodiments, the limiting deflection angle and/or the limiting delay time of the transmission beam in the first hybrid wave imaging mode and/or the second hybrid wave imaging mode may be determined by the calculation of the array element directional function. In some embodiments, an array element directional restriction condition of the first hybrid wave imaging mode and an array element directional restriction condition of the second hybrid wave imaging mode may be the same. In some embodiments, the array element directional restriction condition of the first hybrid wave imaging mode and the array element directional restriction condition of the second hybrid wave imaging mode may be different. In some embodiments, the limiting deflection angle and/or the limiting delay time of the transmission beams in the first hybrid wave imaging mode and the limiting deflection angle and/or the limiting delay time of the transmission beams in the second hybrid wave imaging mode may be the same. In some embodiments, the limiting deflection angle and/or the limiting delay time of the transmission beams in the first hybrid wave imaging mode and the limiting deflection angle and/or the limiting delay time of the transmission beams in the second hybrid wave imaging mode may be different.

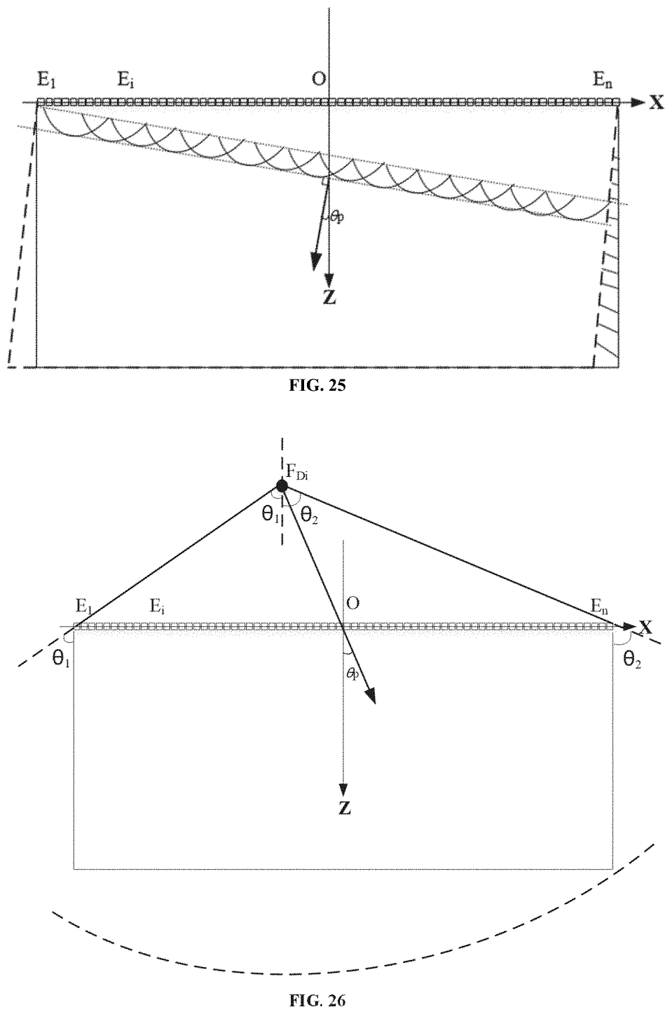

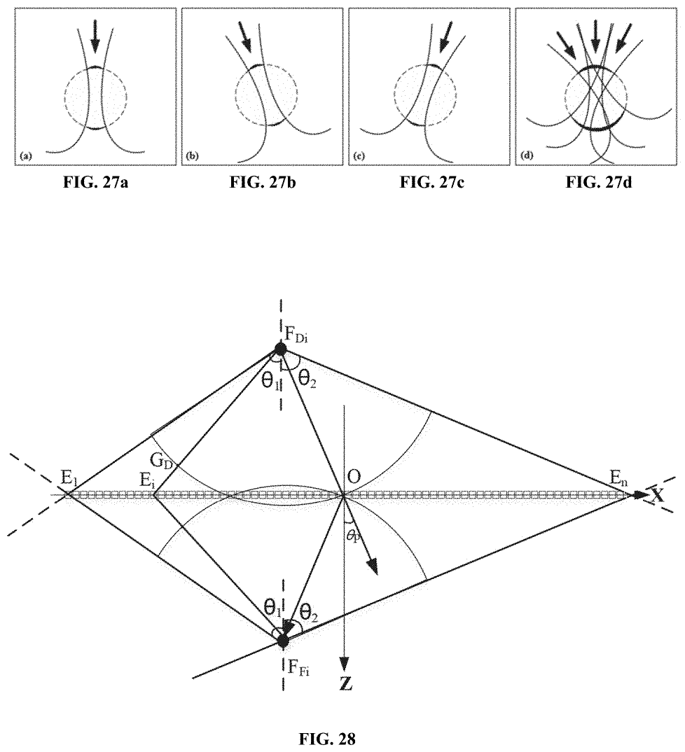

FIG. 25 and FIG. 26 are schematic diagrams illustrating determining a limiting deflection angle and a limiting delay time according to an array element directional restriction condition according to some embodiments of the present disclosure. FIGS. 27a-27d are effect schematic diagrams illustrating an exemplary deflection scanning using divergent wave beams according to some embodiments of the present disclosure. FIG. 28 is a schematic diagram illustrating determining a limiting deflection angle and a limiting delay time according to an array element directional restriction condition according to some embodiments of the present disclosure. It should be noted that a calculation manner of the limiting deflection angle and the limiting delay time in the examples shown in FIG. 25 to FIG. 28 may be used in the first hybrid wave imaging mode and/or the second hybrid wave imaging mode.

Specifically, as shown in FIG. 25 and FIG. 26, taking the plane wave beam and divergent wave beam as examples respectively, when an orientation surface of the array element array and an acoustic beam is scanned on an XOZ plane, a unified excitation signal may be applied to each array element, and the direction of a main lobe acoustic beam of plane beams may be consistent with a positive direction of the Z axis, at this time, a deflection angle of the acoustic beam may be 0. If a delay time of equal time difference is applied to adjacent array elements, then the direction of the plane beam may be deflected, and a deflection angle formed by the beam direction and a normal of the array may be Op. An acoustic field directivity function Ds of a beam generated by the array element may be expressed by Equation (7):

$$D s = \frac{sin [ \frac{π Nd}{λ} ( sin θ - sin {θ}_{p} ) ]}{N sin [ \frac{π d}{λ} ( sin θ - sin {θ}_{p} ) ]} , \qquad ( 7 )$$

wherein N may be the count of array elements, d may be an array element spacing, θp may be a beam deflection angle, λ may be a wavelength of the transmitted acoustic wave.

The limiting deflection angle corresponding to a maximum value of directional function Ds may be deduced, which may be calculated by Equation (8):

θi=arcsin(sin θₚ±lλ/d). (8)

When l=0, θp may be a main maximum direction, l=1, 2, . . . , the corresponding θi may be a direction in which the maximum value of each grating lobe appears, so there may be a maximum deflection angle due to the directional restriction when the beam direction is deflected.

In some embodiments, the deflection angle θp may be taken from 0 to 12 degrees (including 12 degrees). In some embodiments, the delay time may be taken between 0 and 20 microseconds (including 20 microseconds).

As shown in FIG. 25, when the plane wave is transmitted vertically in the positive direction of the Z axis, a rectangular solid line region may be the effective imaging region, and when the deflection angle is transmitted, a dotted parallelogram region may be the effective imaging region. Compared with the divergent wave shown in FIG. 26, it may be seen that the divergent wave has a larger effective imaging range than the plane wave by using the same beam deflection angle on the premise of meeting the above array element directional restriction condition, therefore, in some embodiments, the imaging process may preferably be combined with the use of the divergent wave for deflection scanning.

In some embodiments, a deflection angle of the plane wave beam may meet the array element directional restriction condition, so that a main lobe of the beam within the maximum deflection angle may maintain good acoustic field characteristics in the beam deflection direction to reduce the existence of ultrasonic imaging artifacts and improve the imaging quality.

As shown in FIG. 27a to FIG. 27d, if there is a large heterogeneous tissue in a measured medium of the scanning object, the echo signals that may be generated by the transmission beam at different angles on a surface of the medium may be different. In some embodiments, a strong echo signal may be formed on an interface perpendicular to the beam direction in FIG. 27a, FIG. 27b, and FIG. 27c, so a contour perpendicular to the beam in the image may be obvious. If multi angle beams are transmitted and an image is synthesized, the image may include more boundary information, as shown in FIG. 27d. Therefore, in some embodiments, an image generated by line scanning using high-energy focused beams may be used as a basic image, and an image generated using divergent wave beams may be used as an offset image to supplement media boundary information and suppress random noise, which may not only ensure the image quality but also improve the imaging frame rate.

Divergent wave beams and focused wave beams are taken as examples to illustrate the calculation of the limiting deflection angle and the limiting delay time. The delay time shown in FIG. 28 may be a non-zeroing delay time.

Specifically, a delay time of the divergent wave beam may be calculated by Equation (9):

TX_{DivergingDelay}[i]=(F_{Di}Eᵢ−F_{D}_{ᵢ}O)÷c. (9)

A delay time of the focused wave beam may be calculated by Equation (10):

TX_{FocalDelay}[i]=(F_{Fi}O−F_{Fi}Eᵢ)÷C, (10)

wherein Eᵢ may be the ith element in the aperture, F_{D}_{ᵢ} may be a virtual focal point, F_{F}_{ᵢ} may be a real focal point, c may be a speed at which acoustic waves travel through the medium, i.e., an acoustic velocity. When the point O is a transmission aperture center and F_{D}_{ᵢ}O is the transmission beam direction of the divergent wave, F_{F}_{ᵢ}O is a transmission beam direction of the focused wave, an arc with the virtual focal point F_{D}_{ᵢ} and the real focal point F_{F}_{ᵢ} as a center of a corresponding circle may be drawn, respectively, passing the point O, to obtain wavefront reference lines of the diverging wave and the focused wave. A delay time of the array element Eᵢ may be determined based on a normal distance GDEi from the wavefront reference line to the array element and a Z coordinate of a wavefront reference point GD.

Specifically, when calculating the delay time of the diverging wave, if the Z coordinate of GD is negative, the calculated delay time may be negative, then the greater the distance GDEi, the earlier to transmit. If the Z coordinate of GD is positive, the calculated delay time may be positive, then the smaller the distance GDEi, the earlier to transmit, and the formed wavefront may tend to spread in the imaging region. On the contrary, the calculation of the delay time of the focused wave is the opposite, and the formed wavefront may tend to converge in the imaging region.

Assuming that the virtual focal point and the real focal point are symmetrical about the X axis, the full aperture for transmission is used, and a delay reference point is taken as an aperture center, under a condition that the corresponding directional demands are met, since distances from the wavefront to the array elements are the same, a maximum delay time of the focused wave and the divergent wave may be the same after the delay time is reset to zero (i.e., shifted to zero). In other words, the maximum delay time may be independent of the transmission beam types and may be determined by a difference between the maximum value of the non-zeroed delay time, the minimum value of the non-zeroed delay time, a sum of distances from the focal point to the nearest array element and the farthest array element, and a distance from the focal point to the aperture center. Specifically, the limiting delay time after zeroing the divergent wave beam may be calculated by Equation (11):

Delay_{Diverging max}=(EⱼF_{Di}+EₙF_{Di}−2×OF_{Di})÷c. (11)

The limiting delay time after zeroing the focused wave beam may be calculated by Equation (12):

Delay_{Focal max}=(EⱼF_{Fi}+EₙF_{Fi}2×OF_{Fi})÷c, (12)

wherein Eⱼ may be an array element closest to the focal point, Eₙ may be an array element farthest to the focal point.

In some embodiments, in the first hybrid wave imaging mode and/or the second hybrid wave imaging mode, after the limiting deflection angle and/or the limiting delay time are determined, the focal point distribution (e.g., the boundary condition of the first focal point and/or the boundary condition of the second focal point) outside the imaging region may be determined.

In some embodiments, considering the characteristics of the full aperture transmission operation in the first hybrid wave imaging mode, the boundary condition of the first focal point may include: a focal point of the unfocused wave outside the imaging region may be located on a first boundary line segment, a second boundary line segment, and/or a V-shaped region formed by the first boundary line segment and the second boundary line segment which may be far away from the array element array, an extension line of the first boundary line segment and an extension line of the second boundary line segment may respectively pass through a second endpoint of the array element and a first endpoint of the array element, respectively, and an included angle with a straight line perpendicular to the array element may be the limiting deflection angle.

In some embodiments, all focal points of the unfocused wave may be uniformly distributed in the first boundary line segment and the second boundary line segment to ensure that when the unfocused wave is transmitted, the transmission of all focal points of the unfocused wave may perform the full aperture transmission operation under the array element directional restriction condition of the limiting deflection angle or the limiting delay time, so that each focal point may cover a wide scanning range (e.g., covering at least the imaging region or a region larger than the imaging region) each time it is transmitted. In some embodiments, a part of the focal points of the unfocused wave may be arranged at the first boundary line segment and the second boundary line segment and the rest of the focal points may be arranged in the V-shaped region far away from the array element array and surrounded by the first boundary line segment and the second boundary line segment. This arrangement may ensure that part of the focal points of the unfocused wave may be used in the full aperture transmission operation. The unfocused wave has the characteristics of wide coverage, even if only part of the focal points are used in the full aperture transmission, the overall imaging effect may still be improved in terms of the image quality and the frame rate to meet the imaging needs of the user.

In some embodiments, the focus wave may be uniformly or unevenly distributed within the imaging region so that the focal points of the focus wave may be used in the full aperture transmission operation or the local aperture transmission operation at the time of transmission. In some embodiments, the full aperture transmission operation using the focus wave may include: the outermost two real focal points of the focused waves may correspond to the array elements at both ends of the array element array, and all array elements may be set with corresponding transmission real focal points, i.e., an imaging region of the focused waves when performing the full aperture transmission operation may cover all array elements. In some embodiments, when performing the full aperture transmission operation, the focused waves may be transmitted line by line in a predetermined order according to the set real focal points. In some embodiments, when the full aperture transmission operation is performed, the focused waves (transmitted according to the set virtual focal points) may be alternately transmitted with the unfocused waves (transmitted according to the set real focal points).

In some embodiments, the local aperture transmission operation using the focused wave may include: the outermost two real focal points of the focused wave may correspond to the array elements at both ends of the array element array, and only the local (or partial) array elements may be set with corresponding transmission real focal points, i.e., an imaging region of the local aperture transmission operation using the focused wave may cover a specific portion of the array elements. In some embodiments, the local aperture transmission operation using the focused wave may include: the outermost two real focal points of the focused wave may correspond to the non-end array elements of the array, and only the local (or partial) array elements may be set with the corresponding transmission real focal points, i.e., an imaging region of the local aperture transmission operation using the focused wave may cover a specific portion of the array elements. In some embodiments, when performing the local aperture transmission operation, the focused wave may be transmitted line by line (or scanned line by line) in a predetermined order according to the set real focal points. For example, each array element participating in the transmission may transmit line by line according to each real focal point. In some embodiments, when performing the local aperture transmission operation, the focused wave (transmitted according to the set real focal points) may be alternately transmitted with the unfocused wave (transmitted according to the set virtual focal points).

In the first hybrid wave imaging mode, the corresponding position of the focal point may be arranged according to the boundary condition of the first focal point, so that all or part of the focal points of the unfocused wave may cover a wide scanning range under the array element directional restriction condition (e.g., the limiting deflection angle or the limiting delay time is met). Exemplarily, taking echo data transmitted from all focal points of the unfocused wave as a basic image and the echo data transmitted from all focal points of the focused wave as an enhanced image may guarantee the high image quality in the imaging region, greatly reduce the count of the transmission times and the transmission time duration to improve the imaging speed, the frame rate of the overall imaging may also be improved, the overall imaging efficiency may be improved, and the high imaging needs of the user (e.g., moving tissue imaging scenes such as cardiac imaging that requires a high frame rate, etc.) may be met.

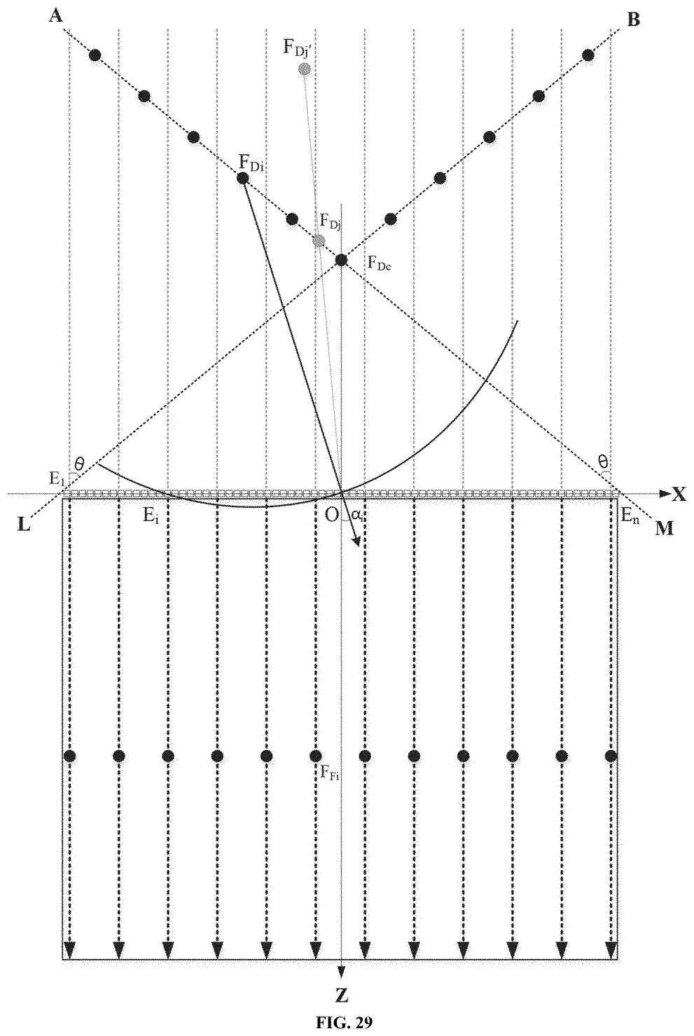

FIG. 29 is a schematic diagram illustrating an exemplary focal point distribution in a hybrid wave imaging mode according to some embodiments of the present disclosure. As shown in FIG. 29, one or more rows of the transducer array elements (i.e., a one-dimensional array element or a multi-dimensional array element) may be arranged along the X axis, and the focal points of the focused wave may be set above the array elements (i.e., below the X axis), and the focal points of unfocused wave (such as the divergent wave) may be set below the array elements (i.e., above the X axis). Exemplarily, the position of the focal point of the first hybrid wave imaging mode, a deflection angle of the focal point, and/or a delay time of the focal point may be determined according to the boundary condition of the first focal point.

As shown in FIG. 29, when using the divergent wave for the transmission, the array element directional restriction condition may determine that sin(|θ|)≤λ/2l may need to be satisfied at an angle where the acoustic wave aliasing does not occur, λ may be a wavelength of the acoustic wave, and l may be a width of the array element in the transducer. In order to obtain a strong echo signal, all array elements may be selected for the full aperture transmission operation. For example, the first boundary line segment and the second boundary line segment for determining the boundary condition of the first focal point may include: an angle between a connecting line AM passing a second endpoint En of the array element and a negative direction of the Z axis may be θ, an angle between a connecting line BL passing a first endpoint E1 of the array element and a negative direction of the Z axis may be θ (i.e., the limiting deflection angle), and an intersection of the connecting lines may be FDc. When an included angle between a line from the focal point to the aperture boundary and the positive direction of the Z axis is less than or equal to the maximum deflection angle, the aperture may cover the effective imaging region, then the intersection FDc may be a critical point, AFDc, and BFDc may be the left and right boundaries respectively, i.e., the first boundary line segment and the second boundary line segment. When the focal point is within a range of the V-shaped region (away from the array elements) surrounded by AFDcB (including a boundary of the first boundary line segment and a boundary of the second boundary line segment), the full aperture transmission operation may be used to cover all the rectangular regions below the array elements. If the focal point is not within the region, an angle between the line from the focal point to the aperture boundary (e.g., vertical lines AE1 and BEn) and the negative direction of the Z axis may be greater than θ.

When the focal point is located within the AFDcB region, such as a focal point FDj′, a connecting line between the focal point and the aperture center O may intersect AM at a focal point FDj, and a distance FDj′O>FDjO. However, the effective imaging region covered by connecting lines between the focal point FDj′O and the boundaries of the transducer array may become smaller, the points on the first boundary line segment and the second boundary line segment being selected as the focal points may ensure that each transmission may cover a large imaging region. Therefore, when setting the focal points, the focal points may be set on the first boundary line segment AFDc and the second boundary line segment BFDc, or the focal point FDj on a line between the set virtual focal point FDj′ and the aperture center O and on the boundary line AFDc may be calculated according to the following equations, and the calculated focal point FDj may be used to calculate the transmission delay time. When transmitting with FDi as the focal point, a direction in which the focal point FDi points to the aperture center O may be a main direction of the transmission beam, an included angle α with the positive direction of the Z axis is a deflection angle of the beam, and different positions of the focal points above the array may be provided with transmission beams with different deflection angles.

Assuming that a coordinate of FDj′ is (XFDj′,YFDj′), the count of probe array elements may be N, and the array element spacing may be d, linear equations associated with OFDj′ and AM may be expressed as Equation (13):

$$\{ \begin{matrix}y = {( {YF}_{Dj}^{′} / {XF}_{Dj}^{′} )}^{*} x \\ y = - tan θ ⋆ x + ( nd / 2 ) / tan θ\end{matrix} . \qquad ( 13 )$$

In some embodiments, the focal points above the array elements (above the X axis) may be set as virtual focal points, and the focal points below the array elements (below the X axis) may be set as real focal points. Exemplarily, as shown in FIG. 29, the count of real focal points corresponding to local array elements may be uniformly arranged in the imaging region, when transmitting waves according to the real focal points, the local aperture transmission operation may be performed line by line or alternately. In some embodiments, the count of deflection angles may be determined according to the count of virtual focal points to set or adjust the amount of boundary information of the medium that may be obtained in one image. In some embodiments, the transmission line density may be set or adjusted according to the count of real focal points, so that the image quality or the frame rate adjustment of a base image may be achieved by receiving a line density, thereby improving a final image.

In some embodiments, the count of aperture array elements may be calculated by Equation (14):

Count of aperture array elements=Focus depth/(Focusing factor×Array element spacing). (14)

In some embodiments, the focused wave beam may be used to perform scanning transmission line by line based on real focal point(s) (e.g., from one end of the array element to the other end, etc.) to form a basic image, and the unfocused wave beam may be used to perform scanning transmission based on virtual focal point(s) to form a superimposed image.

In some embodiments, in the operation 2320, according to the hybrid wave imaging mode, the corresponding hybrid wave imaging operation may be performed to obtain the corresponding imaging result, which may include one or more of the following operations.

The first hybrid wave echo data and/or the second hybrid wave echo data may be compounded, and the compounding operation may include line compounding, spatial compounding, frequency compounding, image compounding, or the like, or a combination thereof.

In some embodiments, one or more of line compounding, spatial compounding, frequency compounding, and image compounding may be performed according to coherence information of the first hybrid wave echo data and/or the second hybrid wave echo data. In some embodiments, one or more of line compounding, spatial compounding, frequency compounding, and image compounding may be performed based on any feasible algorithm or mean, and the embodiments of the present disclosure are not particularly limited.

According to the imaging needs of different users in different imaging scenes, through a reasonable usage of image compounding operations such as line compounding, spatial compounding, frequency compounding, image compounding, etc., more deflection scanning information may be obtained, and random noise may be effectively suppressed, thus improving the imaging efficiency in terms of the imaging quality and the frame rate.

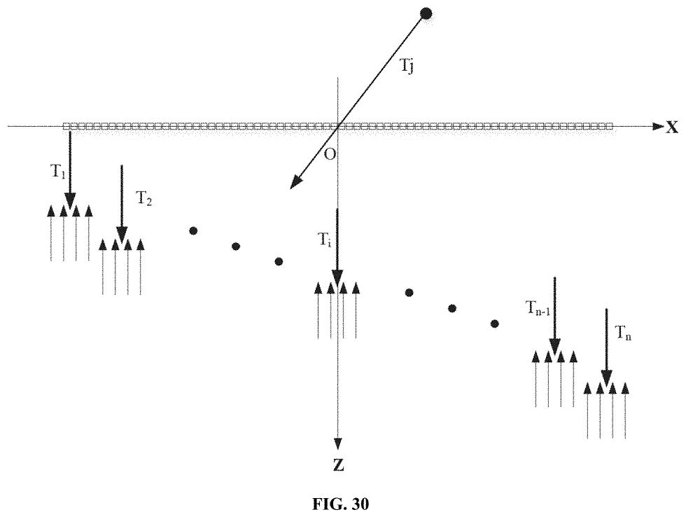

FIG. 30 is a schematic diagram illustrating an exemplary transmission imaging of a first hybrid wave imaging mode according to some embodiments of the present disclosure. In FIG. 29 and FIG. 30, in some embodiments, the focused waves may be transmitted using the local apertures, while the divergent waves may be transmitted using the full apertures. The focused waves may be scanned line by line, and then the divergent waves may be scanned angle by angle. The echo data may be generated by each transmission of focus waves, only m lines may be formed by beamforming (as shown in FIG. 30, m may be taken as 4), each line may correspond to an array element and a real focal point, and all n*m lines may finally form a line scanning image. The divergent waves are transmitted using the full apertures and the array elements cover the effective imaging region, data generated by a single transmission may be used to construct an image (composed of n*m lines of the divergent wave beam).

In some embodiments, the compounding operation of the first hybrid wave echo data may include: considering a correlation between adjacent transmissions, the cumulative weight may be calculated for each transmission, coherent or incoherent recombination may be performed with corresponding weight factors, and/or the image may be obtained. In some embodiments, the coherent recombination or incoherent recombination may be performed on images (i.e., image recombination).

For example, assuming that the count of focal points of the focused waves is n, the count of focal points of the divergent waves is k, each focal point may correspond to one transmission, and the k adjacent transmissions of the divergent waves may have a mutual coverage or overlapping region, the coherent recombination may be performed on demodulated complex data of adjacent transmissions according to the coherence. A weight factor of each pixel may be calculated by using a coherence algorithm such as a phase coherence factor, a symbol coherence factor, and a short order spatial coherence factor, etc. The weight factor may be applied in coherent stacking to exploit coherence between adjacent transmissions, and images generated at all diverging wave declinations (deflection angles) may be compounded into one image. The n adjacent transmissions of the focused beam may involve no coverage or overlap region, and all the lines may be combined into one image. Finally, the divergent wave image and the focused wave image may be incoherently combined in the real number domain to obtain a final image.

In some embodiments, considering the characteristics of the full aperture transmission operation in the second hybrid wave imaging mode, the boundary condition of the second focal point may include: the focal point of the unfocused wave outside the imaging region may be located between a first boundary point and a second boundary point, the first boundary point may be located on a first reference line and a second reference line, an included angle between the first reference line passing through a real focal point of a first end in the imaging region and a vertical line passing through a first endpoint of the array elements may be the limiting deflection angle, and an included angle between the second reference line passing through a second endpoint of the array elements and the vertical line passing through the second endpoint of the array elements may be the limiting deflection angle. The second boundary point may be located on a third reference line and a fourth reference line, an included angle between the third reference line passing through a real focal point of the second end in the imaging region and the vertical line passing through the second endpoint of the array elements may be the limiting deflection angle, and an included angle between the fourth reference line passing through the first endpoint of the array elements and the vertical line passing through the first endpoint of the array elements may be the limiting deflection angle.

In some embodiments, at least part (e.g., all) of the focal points of the unfocused wave may be arranged between the first boundary point and the second boundary point to ensure that when the unfocused wave beam is transmitted, under the array element directional restriction condition (e.g., the limiting deflection angle or the limiting delay time is met), all focal points of the unfocused wave may be used in the moving aperture transmission operation of the local apertures, so that the transmission at each focal point may cover a predetermined scanning range. In some embodiments, the focal points at both ends of the unfocused wave may be arranged at the first boundary point and the second boundary point, and the remaining focal points may be arranged in a region between the first boundary point and the second boundary point. The arrangement may ensure that all focal points of the unfocused wave may be used to perform the local aperture transmission operation. Because the unfocused wave has the characteristics of wide coverage, even if the focal points are used in the moving aperture transmission operation of the local apertures, and the coverage of the imaging region is slightly smaller than that of the full aperture transmission operation, the overall imaging effect may still be improved in terms of the imaging quality and the frame rate, and the imaging needs of the user can be met.

In some embodiments, the focused wave may be uniformly or unevenly distributed within the imaging region so that the focal points of the focused wave may be used to perform the full aperture transmission operation or the local aperture transmission operation at the time of transmission. In some embodiments, using the focused wave to perform the full aperture transmission operation may include: the outermost two real focal points of the focused wave may correspond to the array elements at both ends of the array elements, and all the array elements may be set with corresponding transmission real focal points, i.e., the imaging region of the full aperture transmission operation using the focused wave may cover all the array elements. In some embodiments, when performing the full aperture transmission operation, the focused wave may be scanned line by line in a predetermined order according to the set real focal points. In some embodiments, when performing the full aperture transmission operation, the focused waves (transmitted according to the set real focal points) may be alternately transmitted with the unfocused waves (transmitted according to the set virtual focal points).

In some embodiments, using the focused wave to perform the local aperture transmission operation may include: the outermost two real focal points of the focused wave may correspond to the array elements at both ends of the array element array, and the local (or the partial) array elements may be set with corresponding transmission real focal points, i.e., the imaging region of the local aperture transmission operation using the focused wave may cover a specific part of the array elements. In some embodiments, using the focused wave to perform the local aperture transmission operation may include: the outermost two real focal points of the focused wave may correspond to the non-end array elements of the array, and only the local (or partial) array elements may be set with the corresponding transmission real focal points, i.e., the imaging region of the local aperture transmission operation using the focused wave may cover a specific part of the array elements. In some embodiments, when performing the local aperture transmission operation, the focused waves may be transmitted line by line in a predetermined order according to the set real focal points. In some embodiments, when performing the local aperture transmission operation, the focused waves may be transmitted according to the set real focal points, and the unfocused waves may be transmitted according to the set virtual focal points. In some embodiments, the focused waves and the unfocused waves may be transmitted alternately.

In the second hybrid wave imaging mode, the corresponding positions of the focal points may be arranged according to the boundary condition of the second focal point, so that all or part of the focal points of the unfocused waves may cover a predetermined transmission scanning range under the array element directional restriction condition (e.g., the limiting deflection angle or the limiting delay time is met). Exemplarily, the echo data transmitted from all focal points of the focused wave may be used as a basic image, and the echo data transmitted from all focal points of the unfocused wave may be used as an enhanced image, which may ensure the high imaging quality in the imaging region, reduce the count of transmission times, reduce the transmission time duration to improve the imaging speed, improve the frame rate of the overall imaging and the overall imaging efficiency, and meet the specific imaging needs of the user.

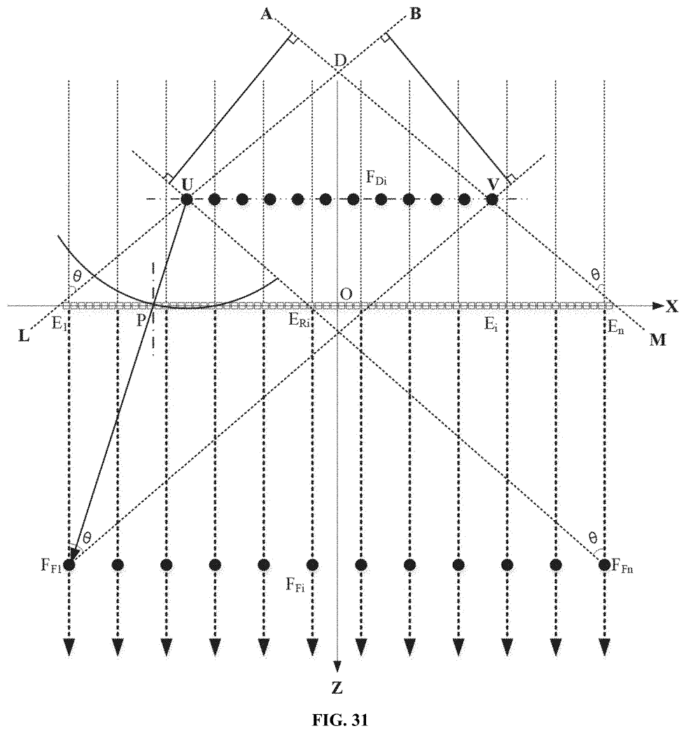

FIG. 31 is a schematic diagram illustrating an exemplary focal point distribution in a hybrid wave imaging mode according to some embodiments of the present disclosure. As shown in FIG. 31, one or more rows of transducer array elements (i.e., one-dimensional array elements or multi-dimensional array elements) may be arranged along the X axis, and the focal points of the focused waves may be located above the array elements (below the X axis). Exemplarily, as shown in FIG. 31, several real focal points corresponding to the local array elements may be uniformly arranged in the imaging region (as shown by the real focal points such as FF1, FFi, FFn, etc., in FIG. 31). When transmitting waves according to the real focal points, the local aperture transmission operation may be performed line-by-line or alternately. Exemplarily, different from transmitting in sequence according to the array element arrangement order of the array elements (such as from left to right), the real focal point cross transmission mode may be adopted according to the alternating of the real focal points. For example, a first real focal point at a leftmost end may transmit a wave first, then a second real focal point may transmit a wave, and so on. The focal points of the unfocused waves (such as the divergent waves) may be located below the array elements (i.e., above the X axis). Exemplarily, the positions of the focal points, the deflection angles of the focal points, or the delay time of the second hybrid wave imaging mode may be determined according to the boundary condition of the second focal point.

As shown in FIG. 31, the focal point of the unfocused wave outside the imaging region (above the X axis) may be located between a first boundary point U and a second boundary point V, the first boundary point U may be located on the first reference line UFFn and the second reference line BL, the first reference line UFFN may pass through a real focal point FFn at a second end in the imaging region and an included angle with a vertical line passing through a second endpoint En of the array elements may be the limiting deflection angle θ, and an included angle between the second reference line BL passing through a first endpoint E1 of the array elements and the vertical line passing through the first endpoint E1 of the array elements may be the limiting deflection angle θ. The second boundary point V may be located on a third reference line FF1V and a fourth reference line AM, the third reference line FF1V may pass through a real focal point FF1 at the first end in the imaging region (below the X axis) and an included angle with a vertical line passing through the first endpoint E1 of the array elements may be the limiting deflection angle θ, and an included angle between the fourth reference line AM passing through the second endpoint En of the array elements and a vertical line passing through the second endpoint En of the array elements may be the limiting deflection angle θ.

Exemplarily, the determination of the first boundary point U and the second boundary point V may include: drawing a straight line E1FF1 perpendicular to the array elements (i.e., the X axis in the figure) through the first endpoint E1 of the array elements, and then take an included angle with the straight line E1FF1 as θ to determine a second reference line BL outside the imaging region (i.e., a negative direction of the Z axis). Drawing a straight line EnFFn perpendicular to the array elements through the second endpoint En of the array elements, and take the included angle with the straight line E1FF1 as θ to determine a fourth reference line AM, a parallel line (i.e., the third reference line FF1V) of the BL passing through the real focus FF1 at the first end (i.e., a leftmost real focal point) makes it intersect with the fourth reference line AM at the point V, a parallel line (i.e., the first reference line UFFn) of AM (i.e., the fourth reference line) passing through the real focus FFn at the second end (i.e., a rightmost real focal point) makes it intersect with the second reference line BL at the point U, and the determined points U and V may be the first and second boundary points of the boundary condition of the second focal point of the unfocused wave. Exemplarily, all the virtual focal points of the unfocused wave may be uniformly or non-uniformly distributed along a line between point U and point V, so that the virtual focal points may cover all the real focal points, i.e., an intersection region below the line UL and VM, under the array element directional restriction condition.

In some embodiments, other virtual focal points may be equally distributed between the points U and V as the first boundary point and the second boundary point. In some embodiments, the count of virtual focal points and the count of real focal points may be the same.

In some embodiments, the leftmost real focal point FF1 and the leftmost virtual focal point U may be connected, and the connecting line and the transducer array intersect at point P. Setting the point P as an aperture center when the unfocused wave is transmitted, the focused wave may be transmitted (using the array element closest to the focal point as the aperture center when the focusing wave is transmitted) vertically downward and focused on the real focal point FF1. That is, a connecting line between the focal point and the aperture center may be a main direction of the beam, if the aperture center of the first transmission line of the focused wave is E1, then E1FF1 may be the main direction of the focused wave, which is vertically downward; if P is the aperture center of the unfocused wave (such as the divergent wave), UP may be the main direction of unfocused wave; if focusing on any virtual focal point between the points U and V, it may be necessary to move the position of aperture center P, so that FDI, P and, FF1 may be in a straight line. In this way, using any virtual focal point between the points U and V (including the points U and V) as the focal point, the central beam of the unfocused wave beam may pass through the real focal point FF1, so that the two beams may form a good sound field distribution at the real focal point.

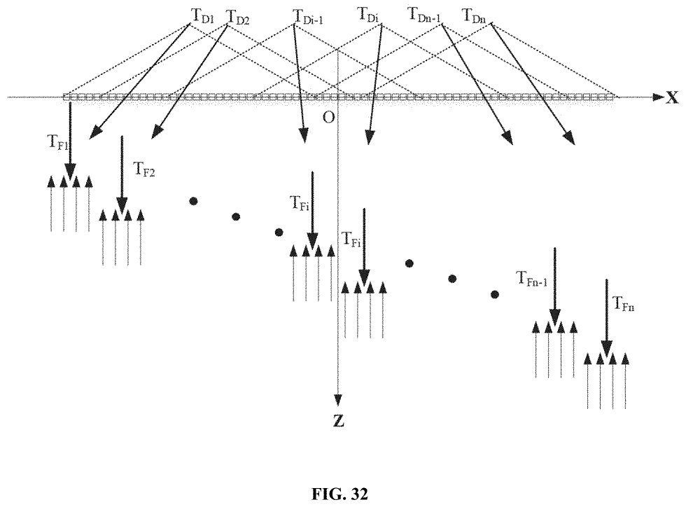

FIG. 32 is a schematic diagram illustrating an exemplary transmission imaging of a second hybrid wave imaging mode according to some embodiments of the present disclosure. In some embodiments, as shown in FIG. 32, the mode of alternately transmitting the focused wave and the divergent wave may be adopted during the transmission. For example, the transmission order of the focal points may be set as: a real focal point TF1, a virtual focal point TD1, a real focal point TF2, and a virtual focal point TD2, etc. During beamforming and/or spatial recombination, a focused beam TFi may form m receiving lines, while the count of scanning lines formed by a divergent beam TDi may be determined by the count of real focal points in the region vertically below the aperture array element. If there are k real focal points under the aperture array element, the divergent beam TDi may synthesize k*m lines at one time.

In some embodiments, the compounding operation of the second hybrid wave echo data may include: the weight factor of the second hybrid wave echo data may be determined according to coherence information of the second hybrid wave echo data; and/or the coherent and/or incoherent compounding may be performed according to the weight factor of the second hybrid wave echo data.

In some embodiments, different from the recombination of the full aperture transmission operation, a main direction center of the beam may pass through the real focal point region when divergent waves are transmitted in the moving aperture transmission operation, and a first coherent recombination manner and/or a second coherent recombination manner may be used for the coherent recombination.

The first coherent recombination manner may adopt the recombination between adjacent transmissions, for example, taking m lines synthesized by TF1, and taking m lines with deflection angle information (i.e., the deflection angle information) at corresponding positions from the k*m lines synthesized by TD1, since these two transmissions are adjacent, the coherence coefficient (i.e., the coherence information) may be calculated for the m lines whose positions overlap (e.g., the phase coherence factor, the symbol coherence factor, the short order spatial coherence factor, etc., may be used, the weight factors may be determined through the coherence factor(s), and then new m lines may be obtained through weighted coherent superposition and compounding as a component of the compounding image. In some embodiments, echo data of adjacent transmissions may be compounded. As shown in FIG. 32, in the example, the focused wave and the divergent wave may be alternately transmitted, and the coherent recombination between the scan lines (i.e., echo data) of the focused wave and the divergent wave may be performed, such as TF1 and TD1, and TD1 and TF2. Therefore, the n real focal points and the n virtual focal points may be compounded for 2*n−1 times, and finally, a compounding image with m*n lines may be synthesized through the coherent recombination.

The second coherent combination manner may adopt the combination between transmissions of the virtual focal points. The divergent wave may form multiple lines each time it is transmitted with the virtual focal points, and there may be cross overlapping regions between different transmissions. The coherence factor of each pixel may be calculated in the overlapping region, and the respective weight factor may be determined through the coherence factor. Then a coherent compounding divergent wave image may be obtained based on the coherence weighting factor. The coherent combination of the divergent wave may suppress the random noise in an anechoic region, and provide different information of the medium under the scanning angle(s).

The image obtained by coherent compounding may include information under multiple deflection angles, and the compounding process may suppress a part of random noise and enhance the amount of information under deflection angle scanning. In some embodiments, on this basis, the two coherent compounding images may be incoherently combined based on corresponding weights, which may further improve the imaging speed of the system, improve the imaging efficiency as a whole, and meet the relevant imaging demands of the user for the imaging quality and the frame rate.

In some embodiments, in operation 2320, the corresponding hybrid wave imaging operation may be performed according to the hybrid wave imaging mode to obtain a corresponding imaging result, which may include one or more of the following operations.

Triggering the hybrid wave imaging operation in the third hybrid wave imaging mode may include: an unfocused wave of a first transmission frequency, a focused wave of a second transmission frequency, and/or a harmonic wave of the third transmission frequency may be transmitted respectively to obtain first unfocused wave imaging data, second focused wave imaging data, and/or third harmonic wave imaging data; and/or performing a coherent and/or incoherent image compounding operation on the first unfocused wave imaging data, the second focused wave imaging data, and/or the third harmonic wave imaging data.

In some embodiments, the hybrid wave imaging operation in the third hybrid wave imaging mode may include presetting two or more (e.g., three) different transmission frequencies (e.g., center frequencies represented by f1, f2, and f3) according to information related to the one or more imaging demands, wherein the wide beam with a central frequency F1 may be transmitted and N1 (N1≥1) image frames may be received. The N1 frames of unfocused wave images may be averaged to obtain the first unfocused wave imaging data; the focused wave beam with a central frequency F2 may be transmitted and image signals may be received to obtain the second focused wave imaging data; two focused wave beams with a same central frequency F3, a same amplitude, and a 180 degree phase difference may be successively transmitted and image signals may be received, and the received signals of the two beams may be summed for harmonic imaging to obtain the third harmonic wave imaging data; and/or coherent and/or incoherent image compounding operations may be performed to obtain the compounded image.

Exemplarily, the hybrid wave imaging operation in the third hybrid wave imaging mode may include one or more of the following operations. The ultrasonic array elements of the probe may successively transmit and receive wide beam signals with a first transmission frequency (such as 7.5 MHz) of N1 frames, image signals of the received N1 frames may be used to construct image(s), and the image signals of the obtained N1 frames may be averaged to obtain an image I1. The ultrasonic array of the probe may transmit focused wave beam signals with a second transmission frequency (e.g., 10 MHz), and image signals may be used to construct an image I2. The ultrasonic array of the probe may successively transmit and receive focused wave beam signals with the same amplitude, the 180 degree phase difference, and the third transmission frequency (e.g., 5 MHz), and image signals may be summed; the summed data may be used to construct an image I3. Incoherent weighted compounding may be performed on the images I1, I2, and I3 to obtain an image Image. In some embodiments, the image Image may be calculated by Equation (15):

Image=w1*l1+w2*l1+w3*l3. (15)

Wherein w1, w2, and w3 may denote the weight factors, and w1, w2, and w3 may be all positive numbers.

In some embodiments, w1, w2, and w3 may be empirical constants determined based on experiences, and their value ranges may be not particularly limited. In some embodiments, the user may adjust the values of w1, w2, and w3 according to specific imaging demands or desired imaging effects. For example, if the user wants to improve the quality of the focal point region while ensuring that the whole image is clear, a difference between w1, w2, and w3 may be set to be small, for example, w1=1, w2=1.5, w3=1.5.

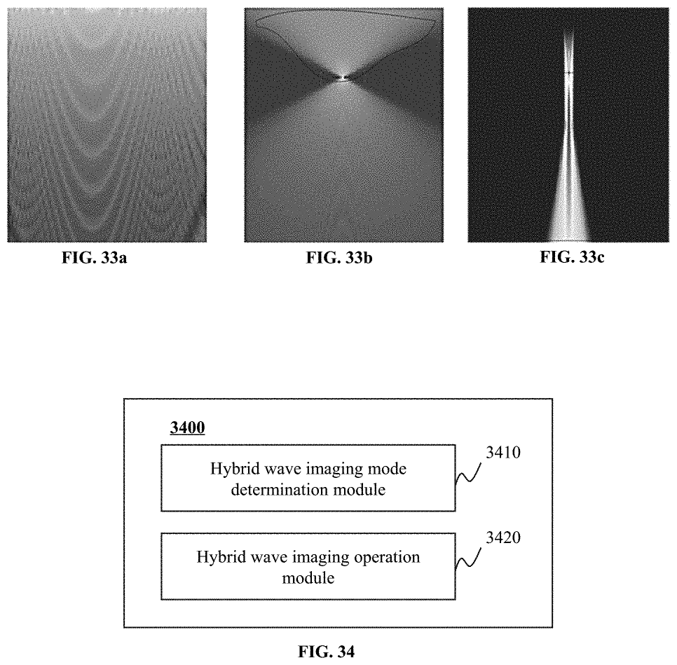

FIGS. 33a-33c are schematic diagrams illustrating an exemplary acoustic pressure distribution of ultrasonic beams in a hybrid imaging mode according to some embodiments of the present disclosure. In some embodiments, the acoustic pressure distribution may be obtained using Field II simulation.

FIG. 33a may show an acoustic pressure distribution of a 7.5 MHz wide beam. FIG. 33b may show an acoustic pressure distribution of a 10 MHz focused beam, a dotted line region in the figure may be a region with large sound pressure, which may mean using the focused beam with larger frequency, the signal attenuation in the shallow region with a small image depth may be small and more information may be retained. FIG. 33c may show an acoustic pressure distribution of a 5 MHz harmonic wave, and a dotted line region in the figure may be a region with large sound pressure. It may be seen that in the harmonic wave imaging mode, a deeper region with a large image depth may have less signal attenuation and more information retention. From the three acoustic pressure distributions, it may be seen that in the hybrid wave imaging mode using hybrid waves with different frequencies, after the image compounding operation, a larger frequency component may improve the imaging quality of the shallow region, and the frequency component of the compounding harmonic wave may improve the spatial resolution of the deep region to reduce speckle noise without losing resolution and improve the imaging quality of the final image; at the same time, since the wide beam transmission may cover the entire imaging region, the echo data of the entire imaging region may be obtained through one transmission and reception. Compared with the traditional focused imaging, it may reduce the count of ultrasonic transmission times and greatly improve the imaging frame rate.

In some embodiments, the personalized imaging quality and the frame rate demands of multiple imaging scenes may be targeted through a variety of combination settings of frequencies and ultrasonic beam types. For example, if more attention is paid to the imaging quality, a hybrid wave with the same beam type (such as the wide beam) but different frequencies may be used, thus greatly improving the imaging operation experiences of the user.

In some embodiments, the tissue motion information of the detection object (such as an organ of the patient) may be obtained, and the parameters of the focused wave and/or the unfocused wave may be determined according to the tissue motion information. In some embodiments, a tracer, or a contrast agent may be used to track the tissue of the detection object to obtain its tissue motion information. In some embodiments, the tissue motion information of the detected object may be detected and obtained through a sensor (such as a position sensor) set on the array elements of the probe. In some embodiments, the tissue motion information of the detection object may include a motion speed of the tissue boundary. In some embodiments, the parameters of the focused wave and/or the unfocused wave may be the count of required transmission focal points of the focused wave and/or the unfocused wave, respectively, and/or the ratio thereof.

In some embodiments, in order to better meet the imaging demands of the specific scene of the user, the imaging quality and/or the frame rate demands may be used as a basis for determining the hybrid wave imaging mode together with tissue motion information of the part to be scanned, or determine the specific imaging demand information of the corresponding imaging quality and/or the frame rate according to the tissue motion information of the part to be scanned (or scanned), and then adjust the hybrid wave imaging mode according to the imaging demand information, such as adjusting the ratio of focused wave transmission times to the unfocused wave transmission times, etc. In some embodiments, the foregoing user specific scenes may include imaging scenes with significant characteristics of tissue motion information, functional tissue imaging scenes with high frame rate demands, such as a cardiography imaging scene, or a blood vessel detection scene.

In some embodiments, the ultrasonic imaging system 100 or the processing device 3400 may adaptively adjust the transmission beam, during imaging, if the position sensor at the array element of the probe detects that the position of the array element of the probe is relatively stable, the echo signals may be generated by transmitting several full aperture divergent waves and analyzed to extract tissue motion speed information at the scanning position. In some embodiments, the frame rate required for the scanning position can be estimated through the selected scanning position and speed information. In some embodiments, the frame rate required for the scanning position may be estimated through the selected scanning position and the speed information. If a higher imaging frame rate is required, the count of focused wave line scans may be reduced and the count of unfocused wave scans may be increased in the subsequent transmission, and a proportion of transmitted focused waves and unfocused waves may be automatically adjusted according to the tissue motion information of the scanning part to realize the automatic control and/or dynamic adjustment of the frame rate.

Exemplarily, in the cardiac contrast imaging scene, the ultrasonic imaging system 100 or the processing device 3400 may obtain the system preset parameters of the matched probe array and/or the part to be scanned. If the array elements are located at the measured site, a position sensor and/or a temperature sensor of the array elements may feedback signals to the system. The system may perform diverging wave imaging at the highest frame rate to obtain tissue images at different times. By detecting the reflection boundary in each frame of image, a displacement s of the tissue boundary may be estimated. Through a displacement distance and the frame interval time t, a movement speed of the tissue boundary V=s÷t may be estimated, i.e., a tissue displacement in unit time. Assuming that a minimum displacement to be tracked by the system is h, and a time to obtain the minimum displacement may be tmin=h/s, a maximum time to form a frame of image may be tmin. Accordingly, the system may ensure that a time to form a frame does not exceed tmin by controlling a ratio of the divergent waves to the focused waves. Assuming that a time required to form the focused wave image is TF and a time required to form the divergent wave image is TD, and the system may control the count of divergent wave images used for compounding to be x and the count of focused wave images to be y, by adjusting the values of x and y, which may ensure that x*td+y*tf<min. However, if a heartbeat of the object is accelerated or slowed down due to some external factors during the scanning interval, i.e., tissue movement information may change, the system may repeat the above process to adjust the automatic frame rate, maximize the performance of system adjustment and optimize the imaging.

In some embodiments, blood flow detection and 2D scanning may require transmission pulses with different center frequencies, so the system may conduct duplex transmission for the two transmissions and process the received data accordingly. Since the two kinds of transmission may not be performed at the same time, and blood flow detection may require multiple transmissions to detect a blood flow, then the detected blood flow may not be consistent with the 2D background in real time.

Exemplarily, in a blood vessel detection scene, the ultrasonic imaging system 100 or the processing device 3400 may use the hybrid transmission to detect blood flow using the divergent waves. Multiple divergent waves may be transmitted to detect the blood flow at at least some points (such as all points) in the effective region covered by the divergent waves, instead of detecting blood flow changes on only one line, which may effectively improve the frame rate of blood flow detection, narrow the time gap with 2D mode, and finally realize fusion and realize the frame rate matching between functional imaging and 2D imaging, and improve the real-time consistency between functional imaging and 2D imaging.

The hybrid wave imaging mode may be applied to corresponding scenes that require a high frame rate or have significant characteristics of tissue motion information. For example, a single transmission of the unfocused wave (such as the divergent wave) may generate a wide range of image data. The combination of the unfocused wave and the focused wave may significantly reduce the count of transmission times of the system in the hybrid wave imaging mode; by using the focused wave to image the whole region or transmitting the focused wave for many times, phase change information of each point in the image with time may also be obtained, which may be used to evaluate a movement of heart tissue or a blood flow of blood vessels, and the image imaging quality may be guaranteed.

FIG. 34 is a block diagram illustrating an exemplary processing device 3400 according to some embodiments of the present disclosure. As shown in FIG. 34, the processing device 3400 may include the hybrid wave imaging mode determination module 3410, and/or the hybrid wave imaging operation module 3420. The hybrid wave imaging mode determination module 3410 may be used to determine the corresponding hybrid wave imaging mode according to the information related to the one or more imaging demands. The hybrid wave imaging operation module 3420 may be used to perform corresponding hybrid wave imaging operations according to the hybrid wave imaging mode and obtain corresponding imaging results. More description about the processing device 3400 executing the ultrasonic imaging process or the hybrid wave imaging mode determination process of the hybrid wave imaging mode determination module 3410 and the hybrid wave imaging operation process of the hybrid wave imaging operation module 3420 may be found in any embodiment above of the ultrasonic imaging process 2300 and the related descriptions, which may not be repeated here.

The ultrasonic imaging system provided by some embodiments of the present disclosure may include: at least one storage medium storing a set of instructions; at least one processor in communication with the at least one storage medium, when executing the stored set of instructions, the at least one processor causes the system to: determine corresponding hybrid wave imaging mode according to information related to the one or more imaging demands; obtain corresponding imaging result by performing corresponding hybrid wave imaging operation according to the hybrid wave imaging mode; and wherein the one or more imaging demand at least include a demand related to the imaging quality and/or the frame rate, the hybrid wave imaging operation at least includes utilizing at least two different transmission beam types and/or at least two transmission frequencies for imaging, and the transmission beam types may at least include a focused wave and/or an unfocused wave.

Further, some embodiments of the present disclosure also provide an ultrasonic imaging device, including a processor, which may be used to execute the corresponding operations of the ultrasonic imaging process 2300 as described in any one of the foregoing embodiments. More descriptions may be found in FIGS. 23-33 and the related descriptions, which may not be repeated here.

Further, some embodiments of the present disclosure also provide a non-transitory computer readable medium including executable instructions, the instructions, when executed by at least one processor, causing the at least one processor to execute the corresponding operations of the ultrasonic imaging process 2300 as described in any one of the foregoing embodiments. More descriptions may be found in FIGS. 23-33 and the related descriptions, which may not be repeated here.

The possible beneficial effects of the ultrasonic imaging method, system, equipment, and computer-readable storage medium provided by some embodiments of the present disclosure may include but are not limited to: (1) by comprehensively considering the concerned imaging needs of the user including the imaging quality and/or the frame rate, an effective resource allocation may be performed through the use of hybrid wave transmission and multiple compounding means to determine a best hybrid wave imaging mode, and then the imaging operation may be completed in the hybrid wave imaging mode to obtain an optimized imaging result, which may improve the overall imaging efficiency, meet the expected imaging needs of the user, and greatly improve the user experiences; (2) through the full aperture hybrid transmission operation in the hybrid wave imaging mode, a large range of scanning region may be covered, while the frame rate is improved due to a fast imaging speed of the unfocused wave, a wide coverage region, an uniform sound field, and a small count of transmission times, the imaging quality may be improved through the enhancement of focusing wave energy; (3) through the moving aperture operation mode in the hybrid wave imaging mode, it may not only perform transmission scanning in important regions (such as the regions of interest) according to the imaging needs of the user, but also obtain the hybrid wave echo data with richer information, which may be convenient for subsequent echo signal compounding or image compounding processing, and may provide guarantee for meeting the personalized imaging needs of different users in different scenes; (4) according to the imaging needs of different users in different imaging scenes, through the reasonable configuration of image compounding operations such as line compounding, spatial compounding, frequency compounding, image compounding, etc., more scanning information at deflection angles may be obtained, and random noise may be effectively suppressed, thus improving the imaging efficiency in terms of the imaging quality or the frame rate.

The possible beneficial effects of the ultrasonic imaging method, system, equipment, and computer-readable storage medium provided by some embodiments of the present disclosure may include but are not limited to:

(1) By comprehensively considering the mutual influence factors affecting the overall imaging efficiency, including the imaging quality and/or the frame rate, and from a perspective of attention of the user to the imaging needs, an effective resource allocation may be performed to determine a best hybrid wave imaging mode according to the imaging needs of different imaging conditions through the optimized imaging, hybrid wave transmission and the combination thereof in local imaging regions to provide a corresponding imaging scheme with high adaptability and obtain the optimized imaging result, which may not only improve the overall imaging efficiency, but also meet the various expected imaging needs of the user, and greatly improve the user experiences; (2) according to the imaging demands or diagnostic demands of different imaging conditions, performing analyzing and determination according to the corresponding imaging condition data of the selected ROI, determining the target optimized imaging mode based on the imaging condition data, and triggering the corresponding optimized imaging mode to provide a corresponding imaging scheme with high adaptability, which may not improve the imaging efficiency, but also meet a variety of imaging needs, so as to provide a best imaging scheme while overcoming the defects of previous technical schemes; (3) supporting an adaptive enhancement of the imaging mode of the global region in which the region of interest and other regions outside it may be dynamically displayed and adjusted at the same time may make it easier for the user to view and adjust the image during detection or diagnosis, and greatly improve the user experiences; (4) since the calculation involved in the ultrasonic imaging process, such as selection of the region of interest or calculation of imaging condition data, may be calculated and stored in a preset way, and may also provide an interactive mode only when the user needs it for dynamic adjustment before calculation, which may not only ensure a small amount of calculation, but also meet the real-time interaction of the user and dynamic adjustment demands during imaging, and further improves the user experiences; (5) through the full aperture hybrid transmission operation in the hybrid wave imaging mode, a large range of scanning region may be covered, while the frame rate may be improved due to the fast imaging speed of the unfocused wave, the wide coverage region, the uniform sound field, and the small count of transmission times, the imaging quality may be improved through the enhancement of the focused wave energy; (6) through the moving aperture operation in the hybrid wave imaging mode, it may not only perform favorable transmission scanning in the focused region according to the imaging needs of the user, but also obtain more abundant hybrid wave echo data, which may be convenient for the subsequent echo signal compounding or the image compounding processing to provide the guarantee for meeting the personalized imaging needs of different users in different scenes; (7) according to the imaging needs of different users in different imaging scenes, through the reasonable usage of image compounding operations such as line compounding, spatial compounding, frequency compounding, image compounding, etc., more deflection scanning information may be obtained, and random noise may be effectively suppressed, thus improving the imaging efficiency in terms of the imaging quality and the frame rate.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by this disclosure and are within the spirit and scope of the exemplary embodiments of this disclosure.

Meanwhile, certain terminology has been used to describe embodiments of the present disclosure. For example, the terms “one embodiment,” “an embodiment,” and/or “some embodiments” mean that a particular feature, structure, or characteristic described in connection with the embodiment is in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in smaller than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A method for ultrasonic imaging, implemented on a computing device having at least one processing device and at least one storage device, the method comprising:
determining a target imaging mode according to information related to one or more imaging demands; and
obtaining a target imaging result by performing an imaging operation according to the target imaging mode;
wherein the one or more imaging demands at least include a demand related to an imaging quality and/or a frame rate, the target imaging mode includes a first target imaging mode and/or a second target imaging mode, the first target imaging mode is configured to perform an optimized imaging for a local imaging region and/or the second target imaging mode is configured to utilize a hybrid wave with at least two transmission beam types and/or at least two transmission frequencies for imaging.

2. The method of claim 1, wherein the determining a target imaging mode according to information related to one or more imaging demands includes:
obtaining the information related to the one or more imaging demands according to one or more preset demand parameters and/or one or more user inputting instructions; and
determining the target imaging mode from the first target imaging mode and/or the second target imaging mode according to the information related to the one or more imaging demands.

3. The method of claim 2, wherein
the first target imaging mode includes at least one of a first optimized imaging mode or a second optimized imaging mode, the first optimized imaging mode is configured to perform an operation for adjusting a transmission parameter of a transmission covering a global imaging region, the global imaging region includes the local imaging region, and the second optimized imaging mode is configured to perform a synchronous operation of an enhanced imaging covering the local imaging region and a non-enhanced imaging covering the global imaging region, and/or
the second target imaging mode includes at least one of a first hybrid wave imaging mode or a second hybrid wave imaging mode, the first hybrid wave imaging mode is configured to perform an operation of a full aperture hybrid transmission, and the second hybrid wave imaging mode is configured to perform an operation of a moving aperture hybrid transmission.

4. The method of claim 3, wherein the determining a target imaging mode according to information related to one or more imaging demands includes:
determining imaging condition data according to the local imaging region; and
determining the target imaging mode from the first optimized imaging mode and the second optimized imaging mode according to the imaging condition data.

5. The method of claim 4, wherein the determining the target imaging mode from the first optimized imaging mode and the second optimized imaging mode according to the imaging condition data includes:
in response to the imaging condition data satisfying a first condition, designating the first optimized imaging mode as the target imaging mode; and
in response to the imaging condition data not satisfying the first condition, designating the second optimized imaging mode as the target imaging mode.

6. The method of claim 3, wherein the target imaging mode is the second target imaging mode, and the obtaining target imaging result by performing an imaging operation according to the target imaging mode includes:
performing, according to a first transmission parameter of the first hybrid wave imaging mode, a full aperture transmission operation of a first ultrasonic wave, and a full aperture transmission operation or a local aperture transmission operation of a second ultrasonic wave, to obtain first hybrid wave echo data, and determining the target imaging result based on the first hybrid wave echo data; and/or
performing operation of the first ultrasonic wave, and a full aperture transmission operation or a local aperture transmission operation of the second ultrasonic wave, to obtain second hybrid wave echo data, and determining the target imaging result based on the second hybrid wave echo data.

7-15. (canceled)

16. A method for ultrasonic imaging, implemented on a computing device having at least one processing device and at least one storage device, the method comprising:
determining a hybrid wave imaging mode according to information related to one or more imaging demands; and
obtaining an imaging result by performing a hybrid wave imaging operation according to the hybrid wave imaging mode;
wherein the one or more imaging demands at least include a demand related to an image quality and/or a frame rate, the hybrid wave imaging operation at least includes utilizing a hybrid wave with at least two transmission beam types and/or at least two transmission frequencies for imaging, and the transmission beam types at least include a focused wave and/or an unfocused wave.

17. The method of claim 16, wherein the hybrid wave imaging mode includes a first hybrid wave imaging mode and/or a second hybrid wave imaging mode, the first hybrid wave imaging mode includes a full aperture hybrid transmission operation, and the second hybrid wave imaging mode includes a moving aperture hybrid transmission operation.

18. The method of claim 16, wherein the hybrid wave imaging mode includes a first hybrid wave imaging mode and a second hybrid wave imaging mode, and the obtaining an imaging result by performing a hybrid wave imaging operation according to the hybrid wave imaging mode includes:
performing, according to a first transmission parameter of the first hybrid wave imaging mode, a full aperture transmission operation of a first ultrasonic wave, and a full aperture transmission operation or a local aperture transmission operation of a second ultrasonic wave, to obtain first hybrid wave echo data; and/or
performing, according to a second transmission parameter of the second hybrid wave imaging mode, a moving aperture transmission operation of the first ultrasonic wave, and a full aperture transmission operation or a local aperture transmission operation of the second ultrasonic wave, to obtain second hybrid wave echo data.

19. The method of claim 18, wherein the obtaining an imaging result by performing a hybrid wave imaging operation according to the hybrid wave imaging mode further includes:
performing a compounding operation on the first hybrid wave echo data and/or the second hybrid wave echo data, the compounding operation including at least one of line compounding, space compounding, frequency compounding, or image compounding.

20. The method of claim 19, wherein the compounding operation of the second hybrid wave echo data includes:
determining a weight factor of the second hybrid wave echo data according to coherence information of the second hybrid wave data; and
performing coherent and/or incoherent compounding of the second hybrid wave echo data according to the weight factor of the second hybrid wave echo data.

21. The method of claim 16, wherein the obtaining an imaging result by performing a hybrid wave imaging operation according to the hybrid wave imaging mode includes:
triggering the hybrid wave imaging operation of a third hybrid wave imaging mode according to the hybrid wave imaging mode;
obtain first unfocused wave imaging data, second focused wave imaging data, and third harmonic wave imaging data by transmitting an unfocused wave of a first transmission frequency, a focused wave of a second transmission frequency, and a harmonic wave of a third transmission frequency; and
performing a coherent and/or incoherent image compounding operation on the first unfocused wave imaging data, the second focused wave imaging data, and the third harmonic wave imaging data.

22. The method of claim 21, wherein the hybrid wave imaging operation includes performing imaging by transmitting a focused wave and an unfocused wave, wherein a frequency of the focused wave and a frequency of the unfocused wave are different.

23. The method of claim 16, further comprising:
obtaining tissue motion information of a detection object; and
determining a parameter of the focused wave and/or the unfocused wave according to the tissue notion information.

24. The method of claim 16, wherein the hybrid wave imaging operation further includes performing imaging on different local imaging regions in a global imaging region using hybrid waves including different transmission beam types and/or different transmission frequencies.

25. The method of claim 16, wherein the hybrid wave imaging operation further includes:
performing an operation for adjusting a transmission parameter of a transmission covering a global imaging region; or
performing a synchronous operation of an enhanced imaging covering a region of interest and a non-enhanced imaging covering the global imaging region.

26. A system for ultrasonic imaging, comprising:
at least one storage medium storing a set of instructions;
at least one processor in communication with the at least one storage medium, when executing the stored set of instructions, the at least one processor causes the system to perform operations including:
determining a target imaging mode according to information related to one or more imaging demands; and
obtaining a target imaging result by performing an imaging operation according to the target imaging mode;
wherein the one or more imaging demands at least include a demand related to an imaging quality and/or a frame rate, the target imaging mode includes a first target imaging mode and/or a second target imaging mode, the first target imaging mode is configured to perform an optimized imaging for a local imaging region and/or the second target imaging mode is configured to utilize a hybrid wave with at least two transmission beam types and/or at least two transmission frequencies for imaging.

27-34. (canceled)

35. The system of claim 26, wherein the determining a target imaging mode according to information related to one or more imaging demands includes:
obtaining the information related to the one or more imaging demands according to one or more preset demand parameters and/or one or more user inputting instructions; and
determining the target imaging mode from the first target imaging mode and/or the second target imaging mode according to the information related to the one or more imaging demands.

36. The system of claim 35, wherein
the first target imaging mode includes at least one of a first optimized imaging mode or a second optimized imaging mode, the first optimized imaging mode is configured to perform an operation for adjusting a transmission parameter of a transmission covering a global imaging region, the global imaging region includes the local imaging region, and the second optimized imaging mode is configured to perform a synchronous operation of an enhanced imaging covering the local imaging region and a non-enhanced imaging covering the global imaging region, and/or
the second target imaging mode includes at least one of a first hybrid wave imaging mode or a second hybrid wave imaging mode, the first hybrid wave imaging mode is configured to perform an operation of a full aperture hybrid transmission, and the second hybrid wave imaging mode is configured to perform an operation of a moving aperture hybrid transmission.

37. The system of claim 36, wherein the determining a target imaging mode according to information related to one or more imaging demands includes:
determining imaging condition data according to the local imaging region; and
determining the target imaging mode from the first optimized imaging mode and the second optimized imaging mode according to the imaging condition data.
